(19) United States
(12) Patent Application Publication
(10) Pub. No.: US 2011/0105515 A1
(43) Pub. Date: May 5, 2011

(54) TARGETING THE ONCOPROTEIN NUCLEOPHOSMIN

(75) Inventors: Andrew G. Myers, Boston, MA (US); Jeremy Earle Wulff, Victoria (CA); Romain Siegrist, Allschwil (CH); Carl Friedrich Nising, Sankt Augustin (DE); Kok Ping Chan, Cambridge, MA (US)

(73) Assignee: President and Fellows of Harvard College, Cambridge, MA (US)

(21) Appl. No.: 12/672,415

(22) PCT Filed: Jul. 24, 2008
(86) PCT No.: PCT/US08/70984

§ 371 Date: Dec. 13, 2010

Related U.S. Application Data
(60) Provisional application No. 60/954,393, filed on Aug. 7, 2007.
(60) Provisional application No. 61/050,700, filed on May 6, 2008.

(51) Int. Cl. A61K 31/4995; C07D 487/22; A61P 35/00; C07K 1/00

(52) U.S. Cl. 514/250; 544/338; 544/115; 544/341; 530/409

(57) ABSTRACT

(+)-Avrainvillamide, a naturally occurring alkaloid with antiproliferative activity, is shown to bind to the oncoprotein nucleophosmin. Nucleophosmin is known to regulate the tumor suppressor protein p53 and is overexpressed in many different human tumors. The invention provides methods of modulating nucleophosmin and p53 using (+)-avrainvillamide and analogues thereof. These compounds may provide leads for the development of novel anti-cancer therapies that target nucleophosmin.

### RELATED APPLICATIONS

The present application claims priority under 35 U.S.C. §119(e) to U.S. provisional patent applications, U.S. Ser. No. 61/050,700, files May 6, 2008, and U.S. Ser. No. 60/954,393, filed Aug. 7, 2007, which is incorporated herein by reference.

### GOVERNMENT SUPPORT

This invention was made with United States Government support under grant RO1 CA047148 awarded by the National Institutes of Health and under National Science Foundation Graduate Research Fellowship awarded by the National Science Foundation. The United States government has certain rights in the invention.

### BACKGROUND OF THE INVENTION

Many pharmaceutical agents work by covalently binding to nucleophiles found on their biological targets in vivo. For example, enzyme inhibitors are frequently designed to target and covalently bind to nucleophiles (e.g., thiols of cysteines, hydroxyl groups of serine, threonine, or tyrosine) in the active site of the enzyme. Functional groups that bond covalently to active site nucleophiles, therefore, frequently form the basis for the design of potent and selective enzyme inhibitors. Those functional groups that form covalent bonds reversibly (e.g., carbonyl groups, boronic esters) are especially valuable in pharmaceutical development (for leading references, please see Adams, J. Curr. Opin. Chem. Biol. 6:493, 2002, Lecaille et al. Chem. Rev. 102:4459, 2002; each of which is incorporated herein by reference).

(+)-Avrainvillamide (I) is a natural product of fungal origin with antiproliferative effects in a number of different human cancer cell lines (Fenical et al. U.S. Pat. No. 6,066,635, issued May 23, 2000; Sugie et al. J. Antibiot. 54:911-16, 2001; each of which is incorporated herein by reference).

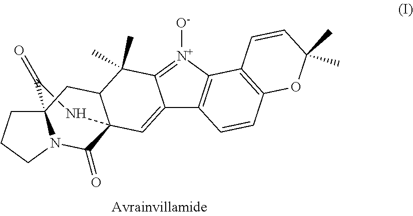

Avrainvillamide includes a 3-alkylidene-3H-indole 1-oxide (unsaturated nitrone) core, which is capable of reversible covalent modification of a heteroatom-based nucleophile. In addition to avrainvillamide's anti-proliferative activity, avrainvillamide has also been reported to exhibit anti-microbial activity against multidrug-resistant bacteria.

Given the anti-proliferative activity of avrainvillamide and its analogues, an effort was made to determine the molecular basis of these effects in hopes of identifying a new target for treating proliferative diseases and designing better modulators of the identified target.

### SUMMARY OF THE INVENTION

Avrainvillamide with its unsaturated nitrone functional group (i.e., 3-alkylidene-3H-indole 1-oxide) has the capacity to bind to multiple nucleophiles in vivo; however, it has been unclear before the present discovery which interactions were responsible for inducing apoptosis in cells treated with avrainvillamide. Based on the use of biotinylated derivatives of avrainvillamide and a simpler analogue of avrainvillamide (see compounds 3 and 4 of FIG. 1), nucleophosmin (also known as numatrin, NO38, and B23) has been discovered to be a principle target of avrainvillamide. It has been further determined that avrainvillamide and its analogues function as electrophiles by reversible, covalent nucleophilic addition of a thiol of nucleophosmin to the unsaturated nitrone core. In particular, further studies have shown that cysteine 275 of nucleophosmin is covalently modified by avrainvillamide and its analogues.

Nucleophosmin is a multifunctional protein that is overexpressed in many human tumors and has been implicated in cancer progression. Nucleophosmin is primarily a nucleolar protein and binds to many different proteins including the tumor suppressor protein p53 (Bertwistle et al. Mol. Cell. Biol. 24:985-96, 2004; Kurki et al. Cancer Cell 5:465-75, 2004; each of which is incorporated herein by reference). It is also frequently mutated in cancer cells. For example, genetic modifications of the C-terminal region of nucleophosmin are common in acute myeloid leukemia (AML) and are believed to be tumorigenic (Falini et al. N. Engl. J. Med. 352:254-66, 2005; Falini et al. Int. J. Cancer 100:662-68, 2002; each of which is incorporated herein by reference). Nucleophosmin has also been found to be deleted in certain tumors (Berger et al. Leukemia 20:319-20, 2006; incorporated herein by reference). Nucleophosmin is thought to be able to regulate p53. RNA silencing of nucleophosmin or disruption of its function by the addition of a small nucleophosmin-binding peptide leads to increased expression of p53 (Chan et al. Biochem. Biophys. Res. Commun. 333:396-403, 2005; incorporated herein by reference).

Based on these discoveries, the present invention provides methods of modifying nucleophosmin by contacting nucleophosmin with avrainvillamide or an analogue thereof. In certain embodiments, the analogue of avrainvillamide useful in the method is of the formula:

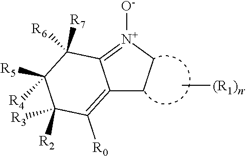

In certain embodiments, nucleophosmin is covalently modified by the compound. In certain embodiments, the analogue of avrainvillamide useful in the method is described in published PCT application, WO2006/102097. The modification of nucleophosmin may be performed in vitro or in vivo. In certain embodiments, the modification is done in a cell (e.g., a malignant cell). The binding event may affect the biological activity or expression of nucleophosmin. The binding of avrainvillamide or an analogue thereof may also affect the expression or biological activity of other nucleophosmin-binding proteins, may affect nucleophosmin's ability to bind polynucleotides, or may affect nucleophosmin's oligomerization state.

In another aspect, the invention provides a method of modulating p53 activity by administering an effective amount of avrainvillamide or an analogue thereof to a cell. Without wishing to be bound by any particular theory, the modulation of p53 is thought to be mediated by covalent modification of nucleophosmin by avrainvillamide or an analogue thereof. Administration of avrainvillamide or an analogue thereof to a cell leads to increased expression of p53. Increased expression of p53 may be useful in the treatment of proliferative diseases such as cancer. Therefore, avrainvillamide and its analogues, such as those described herein and in PCT application, WO 2006/102097, are useful in the treatment of proliferative diseases such as cancer.

In certain embodiments, the invention provides a method of inhibiting the growth of cells by administering an effective amount of avrainvillamide or an analogue thereof. In certain embodiments, the cells are malignant cells. Cells may be treated with avrainvillamide or an analogue thereof in vivo or in vitro. In certain embodiments, the inhibition is performed in a subject such as a human. In certain embodiments, an effective amount of compound is added to the cells to either inhibit the growth of the cells or kill the cells. In certain embodiments, the compound is selective for malignant versus non-malignant cells.

In yet another aspect, the invention provides a method of identifying compounds that bind or modify nucleophosmin. The compounds may or may not be analogues of avrainvillamide. In certain embodiments, the binding or modification of nucleophosmin by the compound modulates the activity of p53. Compounds that target nucleophosmin are useful in the treatment of various proliferative diseases and infectious diseases. Compounds identified using such a screen may be useful in the treatment of proliferative diseases such as cancer. The method involves contacting a test compound with nucleophosmin to determine if the compound has any effect on nucleophosmin. In certain instances, the compound may alkylate nucleophosmin, prevent the phosphorylation of nucleophosmin, or prevent the oligomerization of nucleophosmin. Since these compounds typically covalently modify their target, a labeled derivative of the compound may be used to identify biological targets. The compound may be labeled with a radiolabel, fluorescent tag, biotin tag, or other detectable tag. Identification of compounds in this manner may then be used to refine and develop lead compounds for the treatment of diseases or for probing biological pathways.

In another aspect, the invention provides analogues of avrainvillamide. Compounds of the invention include compounds of the formula:

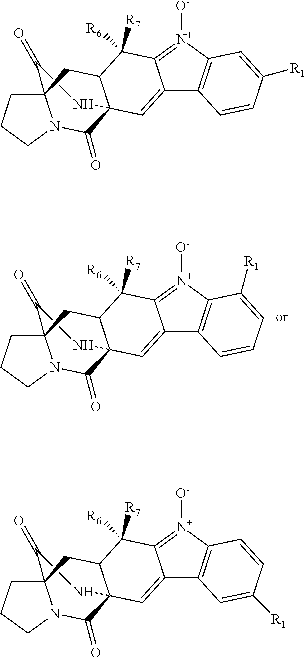

Such compounds include the electrophilic α,β-unsaturated nitrone group of avrainvillamide. These compounds may be used as pharmaceutical agents themselves or may be used as lead compounds in designing new pharmaceutical agents. Particularly, useful compounds are those which exhibit antiproliferative activity or antimicrobial activity. Pharmaceutical compositions and methods of using these compounds to treat diseases such as cancer, inflammatory diseases, autoimmune diseases, diabetic retinopathy, or infectious diseases are also provided.

The invention also provides pharmaceutical compositions of these compounds for use in treating human and veterinary disease. The compounds of the invention are combined with a pharmaceutical excipient to form a pharmaceutical composition for administration to a subject. In certain embodiments, the pharmaceutical composition includes a therapeutically effective amount of the compound. Methods of treating a disease such as cancer or infection are also provided wherein a therapeutically effective amount of an inventive compound is administered to a subject.

The identification of nucleophosmin as a principle biological target of avrainvillamide provides for the identification of antagonists, agonists, or other compounds which bind or modulate the activity of nucleophosmin. The identified compounds are also considered part of the invention.

### Defintions

Definitions of specific functional groups and chemical terms are described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry,” Thomas Sorrell, University Science Books, Sausalito: 1999, the entire contents of which are incorporated herein by reference.

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

One of ordinary skill in the art will appreciate that the synthetic methods, as described herein, utilize a variety of protecting groups. By the term “protecting group”, as used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group should be selectively removable in good yield by readily available, preferably non-toxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen, and carbon protecting groups may be utilized. Hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, trip-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate. Amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide. Exemplary protecting groups are detailed herein. However, it will be appreciated that the present invention is not intended to be limited to these protecting groups; rather, a variety of additional equivalent protecting groups can be readily identified using the above criteria and utilized in the method of the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example, of infectious diseases or proliferative disorders. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl”, and the like. Furthermore, as used herein, the terms “alkyl”, “alkenyl”, “alkynyl”, and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl, and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, vinyl, allyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

The term “alkoxy”, or “thioalkyl” as used herein refers to an alkyl group, as previously defined, attached to the parent molecule through an oxygen atom or through a sulfur atom. In certain embodiments, the alkyl, alkenyl, and alkynyl groups contain 1-20 aliphatic carbon atoms. In certain other embodiments, the alkyl, alkenyl, and alkynyl groups contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups contain 1-6 aliphatic carbon atoms. In yet other embodiments, the alkyl, alkenyl, and alkynyl groups contain 1-4 aliphatic carbon atoms. Examples of alkoxy, include but are not limited to, methoxy, ethoxy, propoxy, isopropoxy, n-butoxy, tert-butoxy, neopentoxy, and n-hexoxy. Examples of thioalkyl include, but are not limited to, methylthio, ethylthio, propylthio, isopropylthio, n-butylthio, and the like.

The term “alkylamino” refers to a group having the structure —NHR′, wherein R′ is aliphatic, as defined herein. In certain embodiments, the aliphatic group contains 1-20 aliphatic carbon atoms. In certain other embodiments, the aliphatic group contains 1-10 aliphatic carbon atoms. In yet other embodiments, the aliphatic group employed in the invention contain 1-8 aliphatic carbon atoms. In still other embodiments, the aliphatic group contains 1-6 aliphatic carbon atoms. In yet other embodiments, the aliphatic group contains 1-4 aliphatic carbon atoms. Examples of alkylamino groups include, but are not limited to, methylamino, ethylamino, n-propylamino, iso-propylamino, cyclopropylamino, n-butylamino, tert-butylamino, neopentylamino, n-pentylamino, hexylamino, cyclohexylamino, and the like.

The term “dialkylamino” refers to a group having the structure —NRR′, wherein R and R′ are each an aliphatic group, as defined herein. R and R′ may be the same or different in an dialkyamino moiety. In certain embodiments, the aliphatic groups contains 1-20 aliphatic carbon atoms. In certain other embodiments, the aliphatic groups contains 1-10 aliphatic carbon atoms. In yet other embodiments, the aliphatic groups employed in the invention contain 1-8 aliphatic carbon atoms. In still other embodiments, the aliphatic groups contains 1-6 aliphatic carbon atoms. In yet other embodiments, the aliphatic groups contains 1-4 aliphatic carbon atoms. Examples of dialkylamino groups include, but are not limited to, dimethylamino, methyl ethylamino, diethylamino, methylpropylamino, di(n-propyl)amino, di(iso-propyl)amino, di(cyclopropyl)amino, di(n-butyl)amino, di(tert-butyl)amino, di(neopentyl)amino, di(n-pentyl)amino, di(hexyl)amino, di(cyclohexyl)amino, and the like. In certain embodiments, R and R′ are linked to form a cyclic structure. The resulting cyclic structure may be aromatic or non-aromatic. Examples of cyclic diaminoalkyl groups include, but are not limited to, aziridinyl, pyrrolidinyl, piperidinyl, morpholinyl, pyrrolyl, imidazolyl, 1,3,4-trianolyl, and tetrazolyl.

Some examples of substituents of the above-described aliphatic (and other) moieties of compounds of the invention include, but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl, and the like. In certain embodiments of the present invention, the term “heteroaryl”, as used herein, refers to a cyclic aromatic radical having from five to ten ring atoms of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups can be unsubstituted or substituted, wherein substitution includes replacement of one, two, three, or more of the hydrogen atoms thereon independently with any one or more of the following moieties including, but not limited to: aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic, or heterocyclic moieties, may optionally be substituted with substituents including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine, and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6-, or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ, wherein each occurrence of Rₓ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

“Carbocycle”: The term “carbocycle”, as used herein, refers to an aromatic or non-aromatic ring in which each atom of the ring is a carbon atom.

“Independently selected”: The term “independently selected” is used herein to indicate that the R groups can be identical or different.

“Labeled”: As used herein, the term “labeled” is intended to mean that a compound has at least one element, isotope, or chemical compound attached to enable the detection of the compound. In general, labels typically fall into five classes: a) isotopic labels, which may be radioactive or heavy isotopes, including, but not limited to, ²H, ³H, ¹³C, ¹⁴C, ¹⁵N, ³¹P, ³²P, ³⁵S, ⁶⁷Ga, ^{99m}Tc (Tc-99m), ¹¹¹In, ¹²³I, ¹²⁵I, ¹⁶⁹Yb, and ¹⁸⁶Re; b) immune labels, which may be antibodies or antigens, which may be bound to enzymes (such as horseradish peroxidase) that produce detectable agents; c) colored, luminescent, phosphorescent, or fluorescent dyes; d) photoaffinity labels; and e) ligands with known binding partners (such as biotin-streptavidin, FK506-FKBP, etc.). It will be appreciated that the labels may be incorporated into the compound at any position that does not interfere with the biological activity or characteristic of the compound that is being detected. In certain embodiments, hydrogen atoms in the compound are replaced with deuterium atoms (²H) to slow the degradation of compound in vivo. Due to isotope effects, enzymatic degradation of the deuterated compounds may be slowed thereby increasing the half-life of the compound in vivo. In other embodiments such as in the identification of the biological target(s) of a natural product or derivative thereof, the compound is labeled with a radioactive isotope, preferably an isotope which emits detectable particles, such as β particles. In certain other embodiments of the invention, photoaffinity labeling is utilized for the direct elucidation of intermolecular interactions in biological systems. A variety of known photophores can be employed, most relying on photoconversion of diazo compounds, azides, or diazirines to nitrenes or carbenes (see, Bayley, H., Photogenerated Reagents in Biochemistry and Molecular Biology (1983), Elsevier, Amsterdam, the entire contents of which are incorporated herein by reference). In certain embodiments of the invention, the photoaffinity labels employed are o-, m- and p-azidobenzoyls, substituted with one or more halogen moieties, including, but not limited to 4-azido-2,3,5,6-tetrafluorobenzoic acid. In other embodiments, biotin labeling is utilized.

“Tautomers”: As used herein, the term “tautomers” are particular isomers of a compound in which a hydrogen and double bond have changed position with respect to the other atoms of the molecule. For a pair of tautomers to exist there must be a mechanism for interconversion. Examples of tautomers include keto-enol forms, imine-enamine forms, amide-imino alcohol forms, amidine-aminidine forms, nitroso-oxime forms, thio ketone-enethiol forms, N-nitroso-hydroxyazo forms, nitro-aci-nitro forms, and pyridone-hydroxypyridine forms.

Definitions of non-chemical terms used throughout the specification include:

“Animal”: The term animal, as used herein, refers to humans as well as non-human animals, including, for example, mammals, birds, reptiles, amphibians, and fish. Preferably, the non-human animal is a mammal (e.g., a rodent, a mouse, a rat, a rabbit, a monkey, a dog, a cat, a primate, or a pig). A non-human animal may be a transgenic animal.

“Associated with”: When two entities are “associated with” one another as described herein, they are linked by a direct or indirect covalent or non-covalent interaction. Preferably, the association is covalent. Desirable non-covalent interactions include hydrogen bonding, van der Waals interactions, hydrophobic interactions, magnetic interactions, electrostatic interactions, etc.

“Nucleophosmin”: The term “nucleophosmin” or “numatrin” or “NO38” or “B23” refers to nucleophosmin polypeptides, proteins, peptides, fragments, variants, and mutants thereof as well as to nucleic acids that encode nucleophosmin polypeptides, proteins, peptides, fragments, variants, or mutants thereof. Nucleophomin has been found to be a biological target of avrainvillamide. Nucleophosmin is a nucleolar protein that plays an important role in ribosome biogenesis and cell proliferation. Nucleophosmin is found to be overexpressed in certain types of tumors. Nucleophosmin may be derived from any species. In certain embodiments, mammalian or human nucleophosmin is referred to.

“Effective amount”: In general, the “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient. For example, the effective amount of a compound with anti-proliferative activity is the amount that results in a sufficient concentration at the site of the tumor to kill or inhibit the growth of tumor cells. The effective amount of a compound used to treat infection is the amount needed to kill or prevent the growth of the organism(s) responsible for the infection.

“Polynucleotide” or “oligonucleotide” refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogues (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, C5-bromouridine, C5-fluorouridine, C5-idouridine, 7-deazaadenosine, 7-deazaguanosine, 8-oxoadenosine, 8-oxoguanosine, O(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

A “protein” or “peptide” comprises a polymer of amino acid residues linked together by peptide bonds. The term, as used herein, refers to proteins, polypeptides, and peptide of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogues as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be just a fragment of a naturally occurring protein or peptide. A protein may be naturally occurring, recombinant, or synthetic, or any combination of these. The terms “protein” and “peptide” encompass glycopeptides and glycoproteins

### BRIEF DESCRIPTION OF THE DRAWING

FIG. 1 shows chemical structures with antiproliferative activities of various inhibitors, activity-based probes, and control compounds.

FIG. 2 are images from fluorescence microscopy experiments with HeLa S3 cells incubated for 2 hours at 37° C. in medium containing 1 μM probe 4 (from FIG. 1), then fixed in methanol. (A) Direct fluorescence observed upon irradiation with 365 nm light, attributed to excitation of the dansyl group of probe 4. (B) Overlay of direct fluorescence output (green) with immunofluorescence output from an antibody to nucleophosmin (red), used here as a nucleolar marker.

FIG. 3 shows Western-blot detection of nucleophosmin after affinity-isolation and PAGE. (A) Affinity-isolation experiments conducted by incubation of probes with living T-47D cells, then lysis. (B) Affinity-isolation experiments with varying concentrations of probe 5 and T-47D whole-cell lysates. (C) Competitive binding studies between the probe 5 and (+)-avrainvillamide (1), (−)-avrainvillamide (ent-1), or analogue 2. (D) Affinity isolation in the absence and presence of idoacetamide.

FIG. 4 shows Western-blot detection of nucleophosmin after affinity-isolation from T-47D nuclear-enriched lysate in the presence of the probe 5 and members of a series of closely related structural analogues of avrainvillamide (1) as competitive binders. The ability of the various compounds to block binding of the probe 5 to nucleophosmin in this experiment parallels their observed potencies in anti-proliferative assays with T-47D cells.

FIG. 5 is a diagram showing the cysteine residues and functional domains present within nucleophosmin (Hingorani et al. J. Biol. Chem. 275:24451-24457, 2000). NPM1.1 is nucleophosmin observed in live cells and cellular lysates. NPM1.3 is a transcript variant employed here for site-directed mutagenesis experiments in COS-7 cells (FIG. 6). The N-terminal non-polar domain is shown in beige; highly acidic regions are shown in blue, moderately basic regions are shown in light green, highly basic clusters are shown in bright green, and the C-terminal region rich in aromatic residues is shown in red. Nuclear and nucleolar signaling regions are indicated in gray.

FIG. 6 shows Western-blot detection of native (NPM1.1) and exogenous (NPM1.3) nucleophosmin in affinity-isolation experiments with 1 μM probe 5. WT=NPM1.3 of unmodified sequence. The presence of native nucleophosmin in the sample lysates constitutes a convenient loading control for the experiment.

FIG. 7 shows (A) increased apoptosis following treatment with (+)-avrainvillamide (1), in HeLa S3 cells depleted in nucleophosmin. Inset shows Western-blot detection of nucleophosmin, following transfection. An estimated 75% depletion in cellular nucleophosmin was observed. (B) Western-blot detection of p53 and nucleophosmin following treatment of live T-47D and LNCaP cells with (+)-avrainvillamide (1) for 24 hours.

FIG. 8 shows fluorescence microscopy experiments with activity-based probe 4 in HeLa S3 cells. (A) Vehicle control reveals background fluorescence. (B) Treatment with 3 μM probe 4 shows both extra- and intranuclear localization. Red arrow indicates a localized concentration of 4 observed inside the nucleus. Data is representative of several cells analyzed.

FIG. 9 shows fluorescence microscopy experiments with activity-based probe 4 in T-47D cells. (A) Vehicle control reveals background fluorescence. (B) Treatment with 1 μM probe 4 shows both extra- and intranuclear localization. Red arrow indicates a localized concentration of 4 observed inside the nucleus. (C) Direct fluorescence from 4 (green) overlaid with immunofluorescent localization of nucleophosmin (red) as a nucleolar marker.

FIG. 10 shows Western-blot detection of peroxiredoxin 1, exportin-1, and nucleophosmin following affinity-isolation experiments in whole-cell lysate.

FIG. 11 shows Western-blot detection of nucleophosmin (and tubulin, as a loading control), 2 days after transfection with two commercially available siRNA reagents (Applied Biosystems, Cat. No. AM16708) or a control siRNA (Applied Biosystems, Cat. No. AM4611). Knockdown was estimated at ˜50% for ID 284660 and ˜75% for ID 143640.

FIG. 12 shows Western-blot detection of p53, nucleophosmin, and 14-3-3β (as a loading control) following lysis of cells treated with increasing concentrations of (+)-avrainvillamide (1).

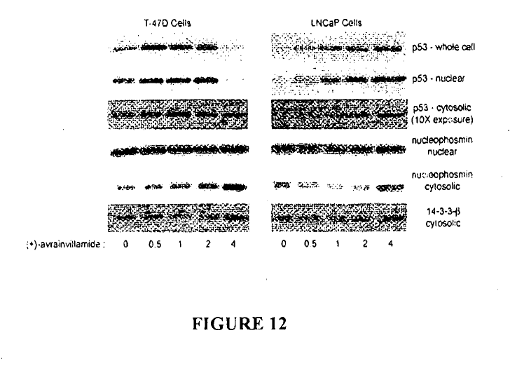

FIG. 13 shows cell cycle accumulatory effects in T-47D cells upon treatment with avrainvillamide. Avrainvillamide causes an immediate decrease in the number of cells in S-phase, followed by an increase in G2/M cells.

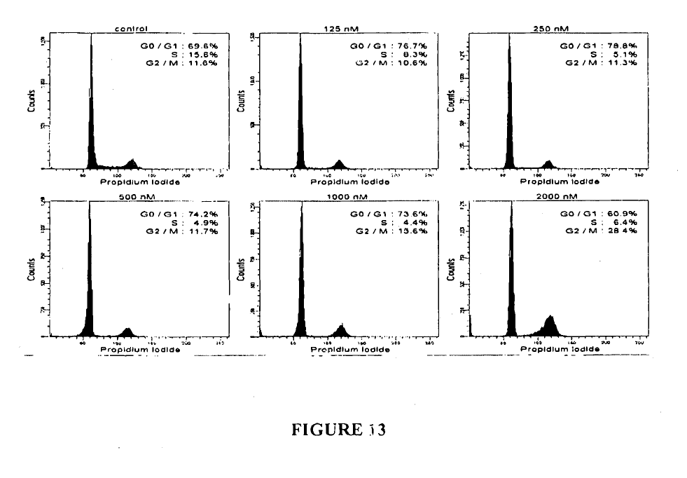

FIG. 14 shows apoptosis data in HeLa S3 cells. The data are plotted using the “density” function in the FloJo software package, to highlight the greatest distinction between cell populations. Dosing HeLa S3 cells with avrainvillamde leads to cell death through apoptosis as shown by Yo-Pro cell permeability experiments and annexin-binding experiments.

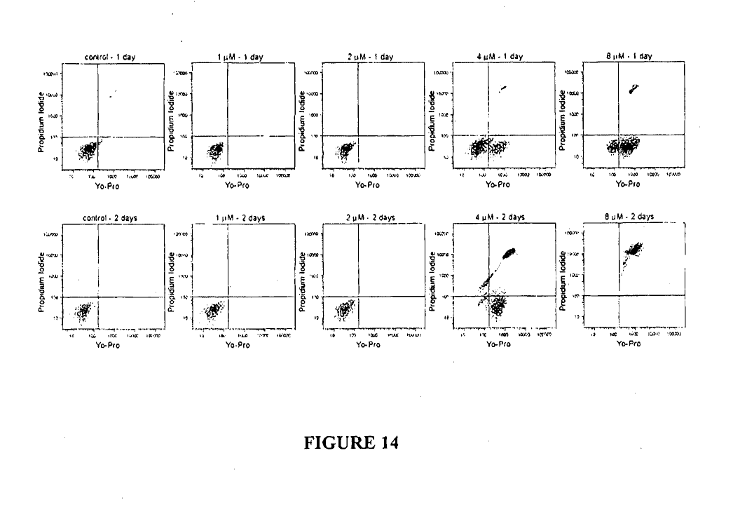

FIG. 15 shows Western blot data for apoptotic markers confirming cell death through apoptosis in LNCaP and T-47D cells. The Western blot data shows the appearance of pro-apoptotic factors with increasing avrainvillamide concentrations.

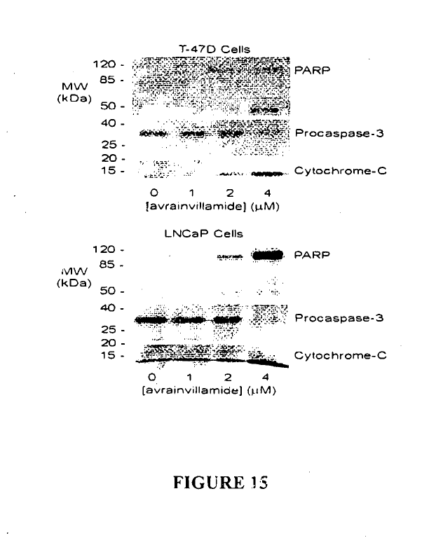

FIG. 16 includes data from a selectivity assay that shows ˜10-fold greater anti-proliferative activity for avrainvillamide in metastatic malignant melanoma than in fibroblast from the same donor.

FIG. 17 includes GI₅₀ data for several analogues of avrainvillamide in the LnCAP (top) and T-47D (bottom) cell lines. LnCap cells are human androgen-sensitive human prostate adenocarcinoma cells, and T-47D are human breast ductal carcinoma cells.

FIG. 18 includes dose response curves for the biphenyl analogue using various cancer cell lines.

FIG. 19 includes dose response curves for the coenzyme A adduct using various cancer cell lines.

FIG. 20 includes dose response curves for the dansyl analogue using various cancer cell lines.

FIG. 21 includes dose response curves for the glutathione adduct using various cancer cell lines.

FIG. 22 includes dose response curves for the deuterated methanol adduct using various cancer cell lines.

### DETAILED DESCRIPTION OF CERTAIN EMBODIMENTS OF THE INVENTION

The present invention stems from the discovery that the oncoprotein nucleophosmin is a principle target for the natural product avrainvillamide. (+)-Avrainvillamide, a naturally occurring alkaloid with anti-proliferative activity, has been found to bind to the nuclear chaperone nucleophosmin, an oncogenic protein that is overexpressed in many different human tumors. Among other biological effects, nucleophosmin is known to regulate the tumor suppressor protein p53. The synthesis of avrainvillamide and analogues thereof was described in published international PCT application, WO 2006/102097, published Sep. 28, 2006; which is incorporated herein by reference.

### Compounds

In one aspect, the present invention provides novel analogues of avrainvillamide. Such compounds may have anti-proliferative and/or anti-microbial activity. The compounds typically include the unsaturated nitrone core functional group (i.e., the 3-alkylidene-3H-indole 1-oxide) of the natural product avrainvillamide.

In certain embodiments, the present invention provides compounds of the formula:

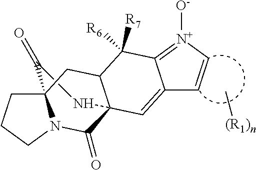

wherein

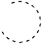

represents a substituted or unsubstituted, cyclic, heterocyclic, aryl, or heteroaryl ring system;

R₁, R₆, and R₇ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

wherein two or more substituents may form substituted or unsubstituted, cyclic, heterocyclic, aryl, or heteroaryl structures;

wherein R₆ and R₇ may form together ═O, ═NR_{G}, or ═C(R_{G})₂, wherein each occurrence of R_{G} is defined as above;

n is an integer between 0 and 4, inclusive; and pharmaceutically acceptable salts, isomers, stereoisomers, enantiomers, diastereomers, and tautomers thereof.

In certain embodiments,

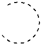

is a monocyclic, bicyclic, tricyclic, or polycyclic ring system, preferably

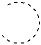

is a monocyclic, bicyclic, or tricyclic ring system. The ring system may be carbocyclic or heterocyclic, aromatic or non-aromatic, substituted or unsubstituted. The ring may include fused rings, bridged rings, spiro-linked rings, or a combination thereof. In certain embodiments,

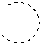

is a monocyclic ring system, preferably a 4-, 5-, 6-, or 7-membered monocyclic ring system, more preferably a 5- or 6-membered ring system, optionally including one, two, or three heteroatoms such as oxygen, nitrogen, or sulfur. In certain embodiments,

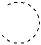

represents a phenyl ring. In other embodiments,

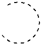

represents a six-member heteroaromatic ring. In other embodiments,

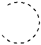

represents a five-member heteroaromatic ring. In yet other embodiments,

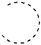

represents a six-membered non-aromatic ring. In still other embodiments,

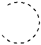

represents a five-membered non-aromatic ring. Examples of particular monocyclic ring systems include:

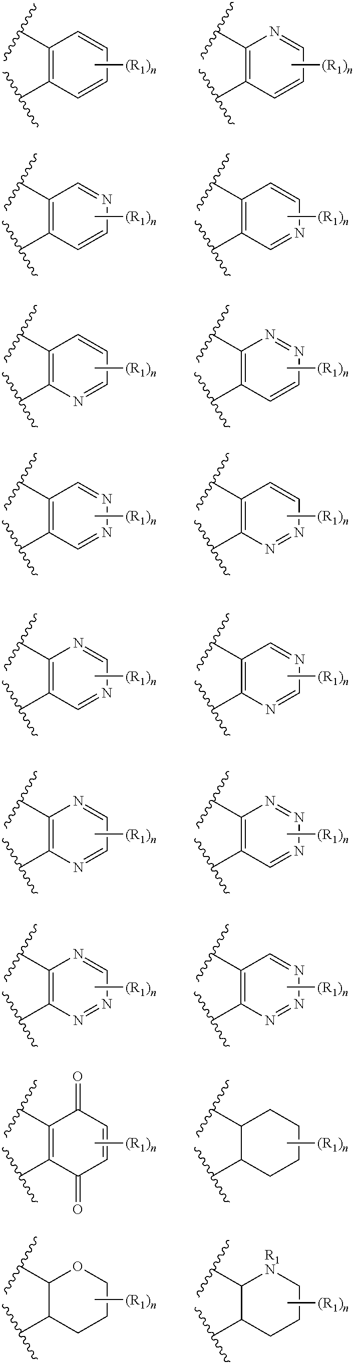

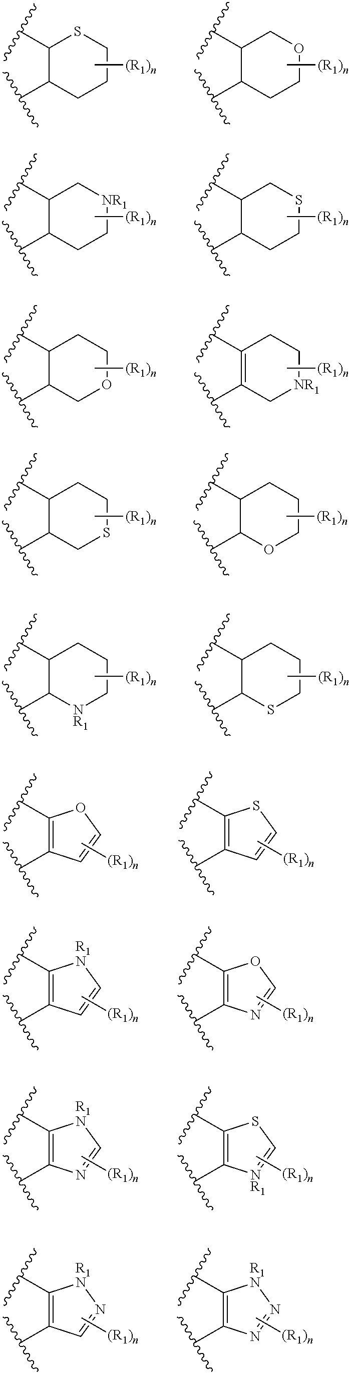

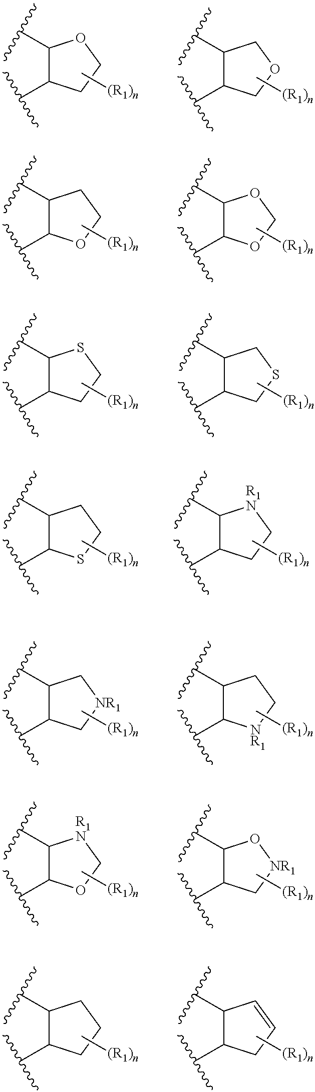

In certain embodiments,

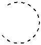

is a phenyl ring with one, two, three, or four substituents, preferably one, two, or three substituents, more preferably one or two substituents. For example,

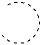

may be

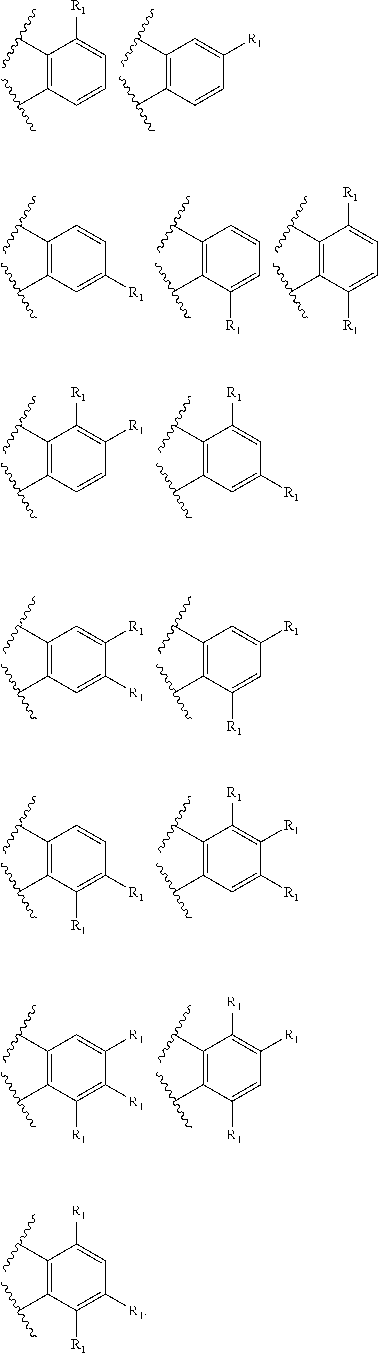

In certain preferred embodiments,

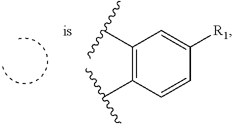

wherein R₁ is —C(R_{G})₃, —OR_{G}, —N(R_{G})₂, or —SR_{G}, wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; preferably R₁ is alkoxy, more preferably methoxy, ethoxy, propoxy, or butoxy. In certain embodiments, R_{G} is an unsubstituted alkyl, alkenyl, or alkynyl group. In certain embodiments, R_{G} is C₁-C₂₀ alkyl. In other embodiments, R_{G} is C₁-C₁₆ alkyl. In yet other embodiments, R_{G} is C₁-C₁₂ alkyl. In still other embodiments, R_{G} is C₁-C₆ alkyl. In certain embodiments, R_{G} is C₁-C₂₀ alkenyl. In other embodiments, R_{G} is C₁-C₁₆ alkenyl. In yet other embodiments, R_{G} is C₁-C₁₂ alkenyl. In still other embodiments, R_{G} is C₁-C₆ alkenyl. In certain embodiments, R_{G} is —(CH₂CH₂O)ₙ—CH₂CH₂OR_{G}′, wherein n is an integer between 0 and 10, and R_{G}′ is hydrogen or C₁-C₆ alkyl (e.g., methyl, ethyl).

In certain embodiments, n is 0. In certain embodiments, n is 1. In certain embodiments, n is 2. In certain embodiments, n is 3. In certain embodiments, n is 4.

In certain embodiments, R₁ is hydrogen; halogen; substituted or unsubstituted aliphatic; substituted or unsubstituted heteroaliphatic; alkoxy; alkylthioxy; acyl; cyano; nitro; amino; alkylamino; or dialkylamino. In certain embodiments, R₁ is hydrogen; halogen; substituted or unsubstituted aliphatic; alkoxy; alkylthioxy; amino; alkylamino; or dialkylamino. In certain embodiments, R₁ is hydrogen, alkoxy, acetoxy, or tosyloxy. In certain embodiments, R₁ is hydrogen or methoxy. In certain embodiments, R₁ is an unsubstituted alkyl, alkenyl, or alkynyl group. In certain embodiments, R₁ is C₁-C₂₀ alkyl. In other embodiments, R₁ is C₁-C₁₆ alkyl. In yet other embodiments, R₁ is C₁-C₁₂ alkyl. In still other embodiments, R₁ is C₁-C₆ alkyl. In certain embodiments, R₁ is methyl. In certain embodiments, R₁ is C₁-C₂₀ alkenyl. In other embodiments, R₁ is C₁-C₁₆ alkenyl. In yet other embodiments, R₁ is C₁-C₁₂ alkenyl. In still other embodiments, R₁ is C₁-C₆ alkenyl. In certain embodiments, R₁ is —(CH₂CH₂O)ₖ—CH₂CH₂OR₁′, wherein k is an integer between 0 and 10, and R₁′ is hydrogen or C₁-C₆ alkyl (e.g., methyl, ethyl). In certain embodiments, R₁ is —OR_{G}, —N(R_{G})₂, or —SR_{G}, wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, R₁ is alkoxy (e.g., methoxy, ethoxy, propoxy, butoxy, etc.). In certain embodiments, R_{G} is an unsubstituted alkyl, alkenyl, or alkynyl group. In certain embodiments, R_{G} is C₁-C₂₀ alkyl. In other embodiments, R_{G} is C₁-C₁₆ alkyl. In yet other embodiments, R_{G} is C₁-C₁₂ alkyl. In still other embodiments, R_{G} is C₁-C₆ alkyl. In certain embodiments, R_{G} is C₁-C₂₀ alkenyl. In other embodiments, R_{G} is C₁-C₁₆ alkenyl. In yet other embodiments, R_{G} is C₁-C₁₂ alkenyl. In still other embodiments, R_{G} is C₁-C₆ alkenyl. In certain embodiments, R_{G} is —(CH₂CH₂O)ₙ—CH₂CH₂OR_{G}′, wherein n is an integer between 0 and 10, and R_{G}′ is hydrogen or C₁-C₆ alkyl (e.g., methyl, ethyl). In certain embodiments, R₁ is substituted or unsubstituted aryl. In certain embodiments, R₁ is substituted or unsubstituted heteroaryl.

In certain embodiments, R₆ is hydrogen. In certain embodiments, R₆ is substituted or unsubstituted aliphatic. In certain embodiments, R₆ is substituted or unsubstituted heteroaliphatic. In certain embodiments, R₆ is substituted or unsubstituted alkyl. In certain embodiments, R₆ is C₁-C₆ alkyl. In certain embodiments, R₆ is methyl. In certain embodiments, R₆ is ethyl. In certain embodiments, R₆ is propyl.

In certain embodiments, R₇ is hydrogen. In certain embodiments, R₇ is substituted or unsubstituted aliphatic. In certain embodiments, R₇ is substituted or unsubstituted heteroaliphatic. In certain embodiments, R₇ is substituted or unsubstituted alkyl. In certain embodiments, R₇ is C₁-C₆ alkyl. In certain embodiments, R₇ is methyl. In certain embodiments, R₇ is ethyl. In certain embodiments, R₇ is propyl.

In certain embodiments, both R₆ and R₇ are hydrogen or C₁-C₆ alkyl. In certain embodiments, both R₆ and R₇ are hydrogen or methyl. In certain embodiments, both R₆ and R₇ are hydrogen. In certain embodiments, both R₆ and R₇ are C₁-C₆ alkyl. In certain embodiments, both R₆ and R₇ are methyl.

In certain embodiments, the present invention provides compounds of the formula:

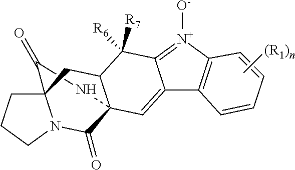

wherein

R₁, R₆, and R₇ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

n is an integer between 0 and 4, inclusive; and pharmaceutically acceptable salts, isomers, stereoisomers, enantiomers, diastereomers, and tautomers thereof.

In certain embodiments, R₆ is hydrogen. In certain embodiments, R₆ is substituted or unsubstituted aliphatic. In certain embodiments, R₆ is substituted or unsubstituted heteroaliphatic. In certain embodiments, R₆ is substituted or unsubstituted alkyl. In certain embodiments, R₆ is C₁-C₆ alkyl. In certain embodiments, R₆ is methyl. In certain embodiments, R₆ is ethyl. In certain embodiments, R₆ is propyl.

In certain embodiments, R₇ is hydrogen. In certain embodiments, R₇ is substituted or unsubstituted aliphatic. In certain embodiments, R₇ is substituted or unsubstituted heteroaliphatic. In certain embodiments, R₇ is substituted or unsubstituted alkyl. In certain embodiments, R₇ is C₁-C₆ alkyl. In certain embodiments, R₇ is methyl. In certain embodiments, R₇ is ethyl. In certain embodiments, R₇ is propyl.

In certain embodiments, both R₆ and R₇ are hydrogen or C₁-C₆ alkyl. In certain embodiments, both R₆ and R₇ are hydrogen or methyl. In certain embodiments, both R₆ and R₇ are hydrogen. In certain embodiments, both R₆ and R₇ are C₁-C₆ alkyl. In certain embodiments, both R₆ and R₇ are methyl.

In certain embodiments, n is 0. In certain embodiments, n is 1. In certain embodiments, n is 2. In certain embodiments, n is 3. In certain embodiments, n is 4.

In certain embodiments, R₁ is substituted or unsubstituted aliphatic. In certain embodiments, R₁ is substituted or unsubstituted heteroaliphatic. In certain embodiments, R₁ is substituted or unsubstituted aryl. In certain embodiments, R₁ is substituted or unsubstituted phenyl. In certain embodiments, R₁ is unsubstituted phenyl. In certain embodiments, R₁ is substituted phenyl. In certain embodiments, R₁ is substituted or unsubstituted heteroaryl. In certain embodiments, R₁ is substituted or unsubstituted pyridyl. In certain embodiments, R₁ is unsubstituted pyridyl. In certain embodiments, R₁ is substituted pyridyl. In certain embodiments, R₁ is arylalkyl. In certain embodiments, R₁ is arylalkenyl. In certain embodiments, R₁ is arylalkynyl. In certain embodiments, R₁ is phenylalkyl. In certain embodiments, R₁ is phenylalkenyl. In certain embodiments, R₁ is phenylalkynyl.

In certain embodiments, the compound is of formula:

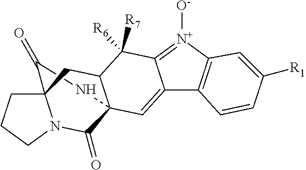

wherein R₁, R₆, and R₇ are defined as above.

In certain embodiments, the compounds is of formula:

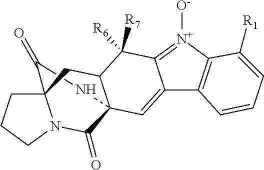

wherein R₁, R₆, and R₇ are defined as above.

In certain embodiments, the compounds is of formula:

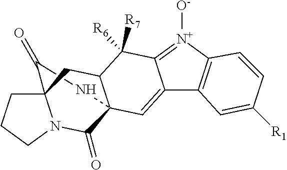

wherein R₁, R₆, and R₇ are defined as above.

In certain embodiments, the compounds is of formula:

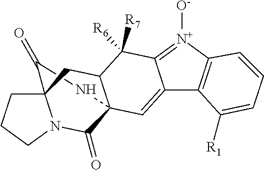

wherein R₁, R₆, and R₇ are defined as above.

Exemplary compounds of the invention include compounds of formula:

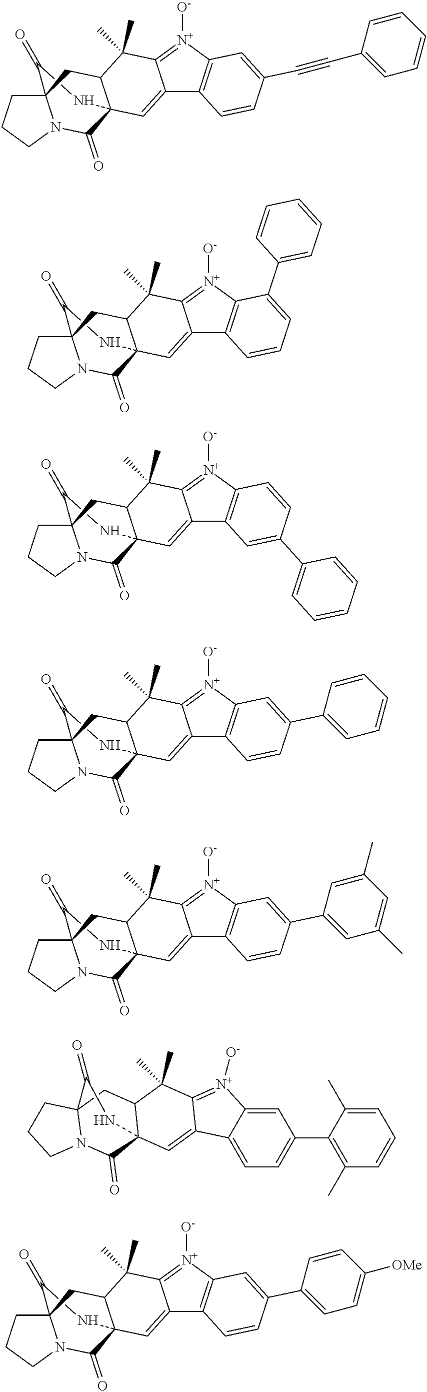

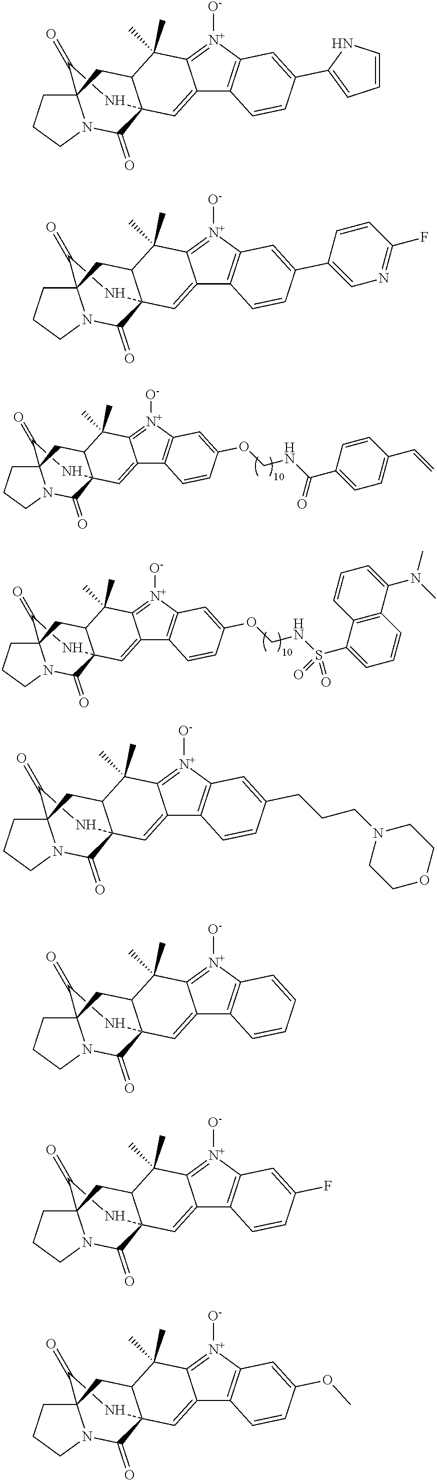

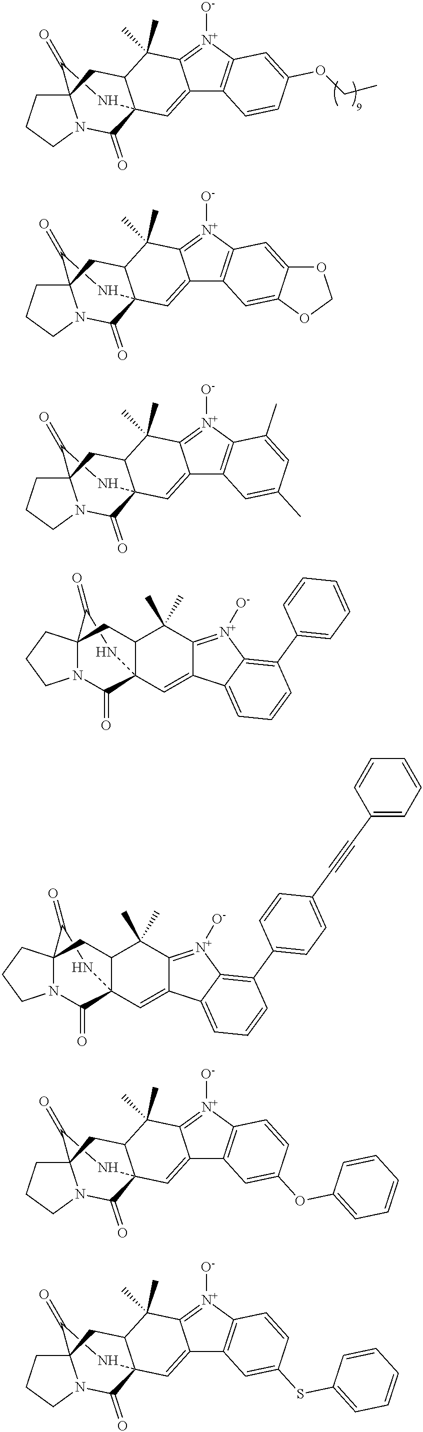

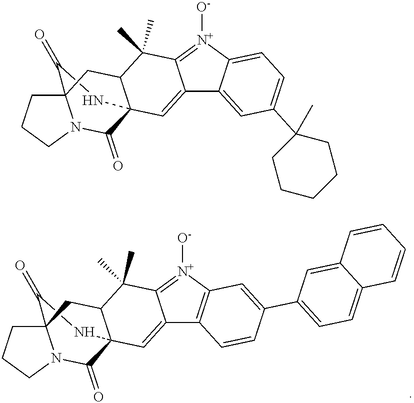

In certain embodiments, the compound is of the formula:

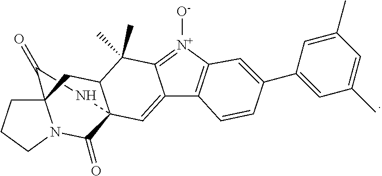

In certain embodiments, the compound is of the formula:

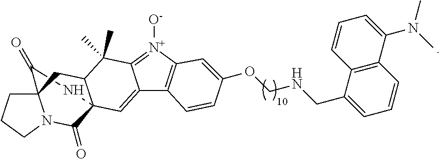

Exemplary compounds of the invention include compounds of formula:

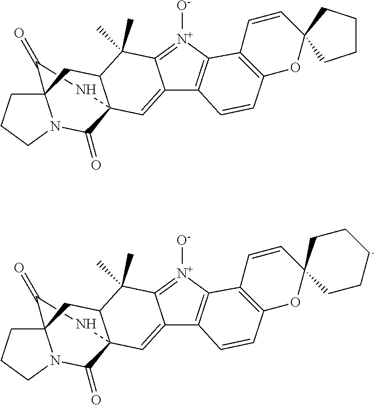

Exemplary compounds of the invention include compounds of formula:

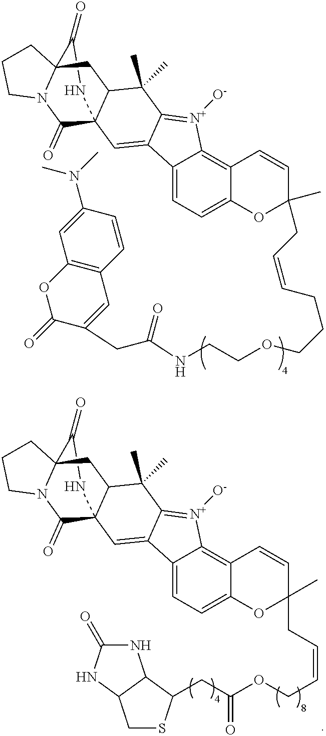

Exemplary compounds of the invention include compounds of formula:

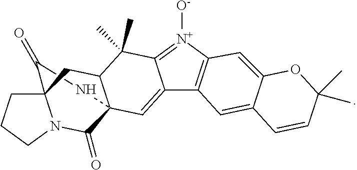

In certain embodiments, the compound is a stereoisomer of formula:

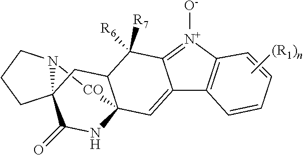

wherein n, R₁, R₆, and R₇ are defined as described herein. In certain embodiments, the compound is of the formula:

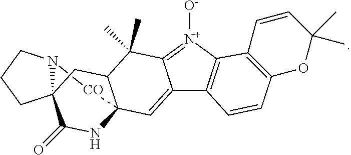

In certain embodiments, a nucleophile such as a thiol or alcohol is added to the α,β-unsaturated nitrone group of an inventive compound by a 1,5-addition to yield a compound of formula:

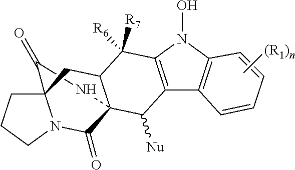

wherein

n, R₁, R₆, and R₇ are defined as described herein; and

Nu is hydrogen, —OR_{Nu}, —SR_{Nu}, —C(R_{Nu})₃, or ˜N(R_{Nu})₂, wherein each occurrence of R_{Nu} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, the compound is of the formula:

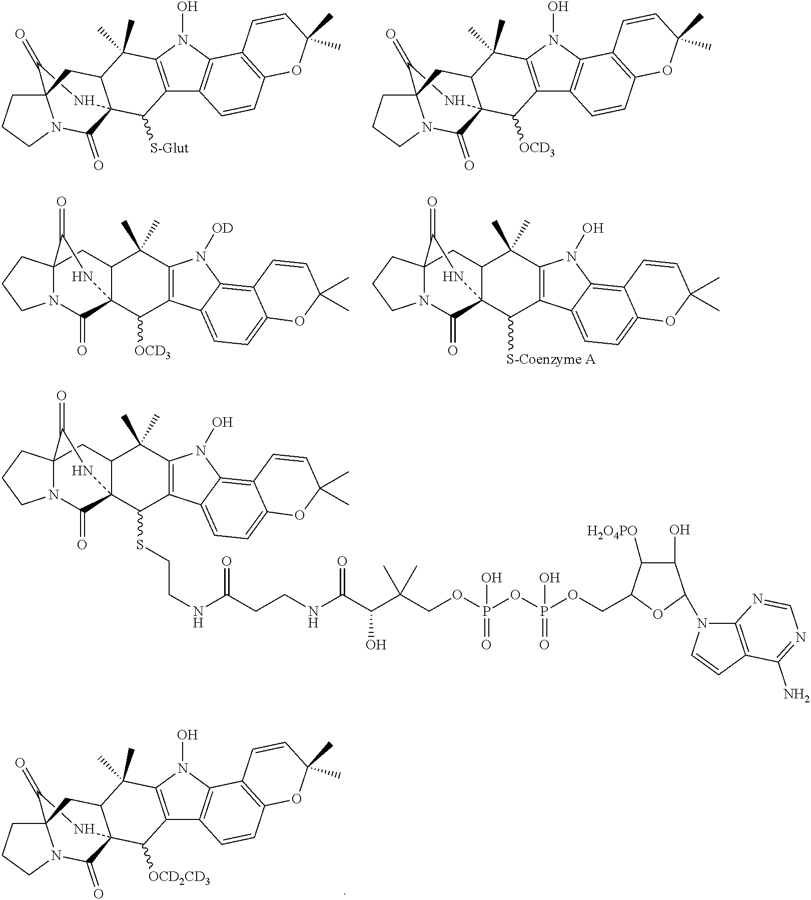

In certain embodiments, the compound is of the formula:

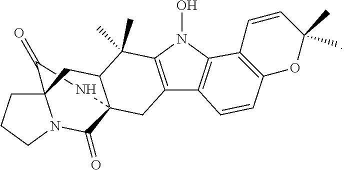

In certain embodiments, the compound is of the formula:

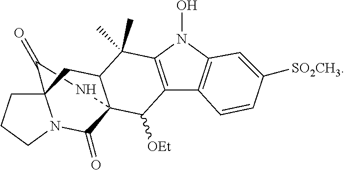

In certain embodiments, the compound is of the formula:

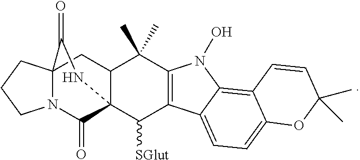

In certain embodiments, the compound is of the formula:

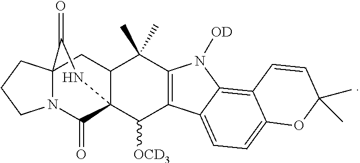

In certain embodiments, the compound is of the formula:

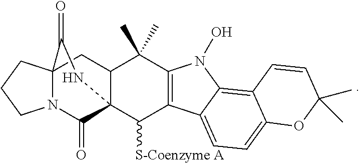

In certain embodiments, the present invention provides compounds of the formula:

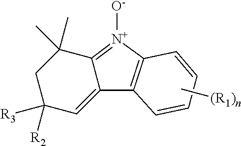

wherein

R₁, R₂, and R₃ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

n is an integer between 0 and 4, inclusive; and pharmaceutically acceptable salts, isomers, stereoisomers, enantiomers, diastereomers, and tautomers thereof.

In certain embodiments, R₂ is hydrogen. In certain embodiments, R₂ is substituted or unsubstituted aliphatic. In certain embodiments, R₂ is substituted or unsubstituted heteroaliphatic. In certain embodiments, R₂ is substituted or unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is —CO₂Me. In certain embodiments, R₂ is amino. In certain embodiments, R₂ is protected amino. In certain embodiments, R₂ is —NHAc. In certain embodiments, R₂ is alkylamino. In certain embodiments, R₂ is dialkylamino.

In certain embodiments, R₃ is hydrogen. In certain embodiments, R₃ is substituted or unsubstituted aliphatic. In certain embodiments, R₃ is substituted or unsubstituted heteroaliphatic. In certain embodiments, R₃ is substituted or unsubstituted alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is acyl. In certain embodiments, R₃ is —CO₂Me. In certain embodiments, R₃ is amino. In certain embodiments, R₃ is protected amino. In certain embodiments, R₃ is —NHAc. In certain embodiments, R₃ is alkylamino. In certain embodiments, R₃ is dialkylamino.

In certain embodiments, both R₂ and R₃ are hydrogen or C₁-C₆ alkyl. In certain embodiments, both R₂ and R₃ are hydrogen or methyl. In certain embodiments, both R₂ and R₃ are hydrogen. In certain embodiments, both R₂ and R₃ are C₁-C₆ alkyl. In certain embodiments, both R₂ and R₃ are methyl. In certain embodiments, both R₂ and R₃ are not methyl. In certain embodiments, both R₂ and R₃ are ethyl. In certain embodiments, both R₂ and R₃ are propyl. In certain embodiments, both R₂ and R₃ are butyl. In certain embodiments, both R₂ and R₃ are the same. In certain embodiments, both R₂ and R₃ are not the same.

In certain embodiments, n is 0. In certain embodiments, n is 1. In certain embodiments, n is 2. In certain embodiments, n is 3. In certain embodiments, n is 4.

In certain embodiments, R₁ is substituted or unsubstituted aliphatic. In certain embodiments, R₁ is substituted or unsubstituted heteroaliphatic. In certain embodiments, R₁ is substituted or unsubstituted aryl. In certain embodiments, R₁ is substituted or unsubstituted phenyl. In certain embodiments, R₁ is unsubstituted phenyl. In certain embodiments, R₁ is substituted phenyl. In certain embodiments, R₁ is substituted or unsubstituted heteroaryl. In certain embodiments, R₁ is substituted or unsubstituted pyridyl. In certain embodiments, R₁ is unsubstituted pyridyl. In certain embodiments, R₁ is substituted pyridyl. In certain embodiments, R₁ is arylalkyl. In certain embodiments, R₁ is arylalkenyl. In certain embodiments, R₁ is arylalkynyl. In certain embodiments, R₁ is phenylalkyl. In certain embodiments, R₁ is phenylalkenyl. In certain embodiments, R₁ is phenylalkynyl.

In certain embodiments, the compound is of formula:

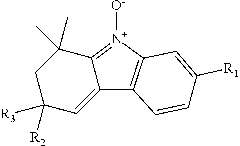

wherein R₁, R₂, and R₃ are defined as above.

In certain embodiments, the compounds is of formula:

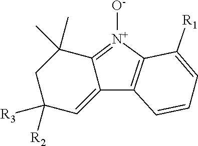

wherein R₁, R₂, and R₃ are defined as above.

In certain embodiments, the compounds is of formula:

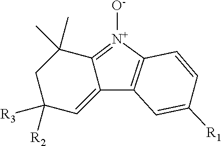

wherein R₁, R₂, and R₃ are defined as above.

In certain embodiments, the compounds is of formula:

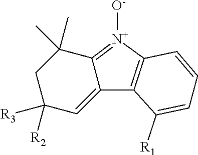

wherein R₁, R₂, and R₃ are defined as above.

Exemplary compounds of the invention include compounds of formula:

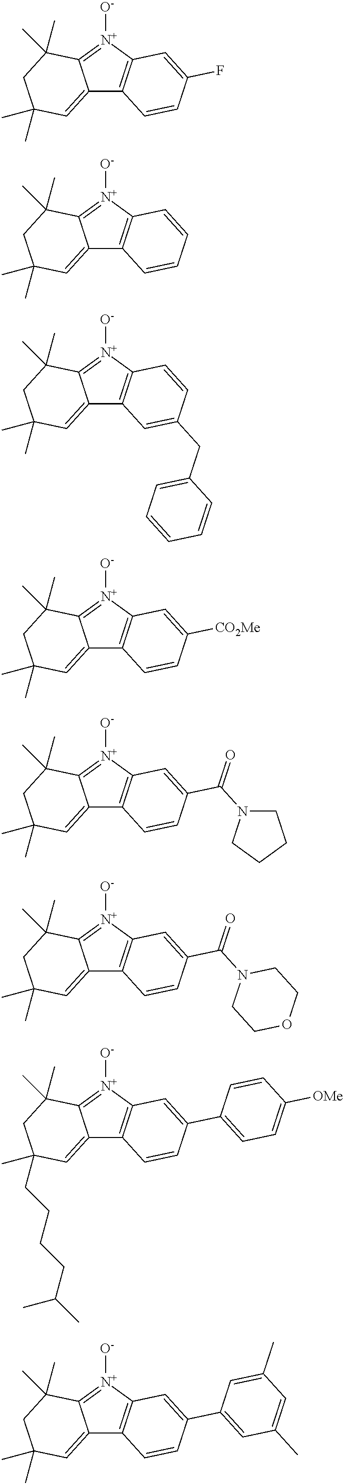

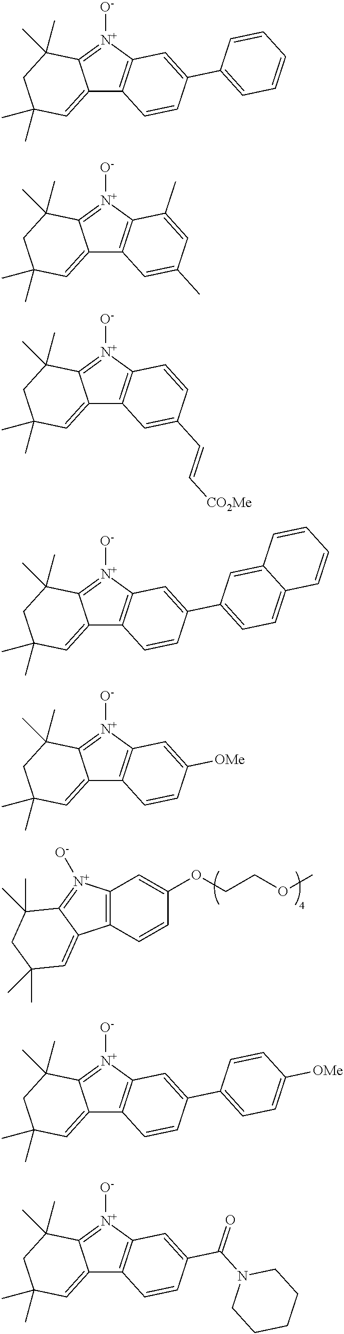

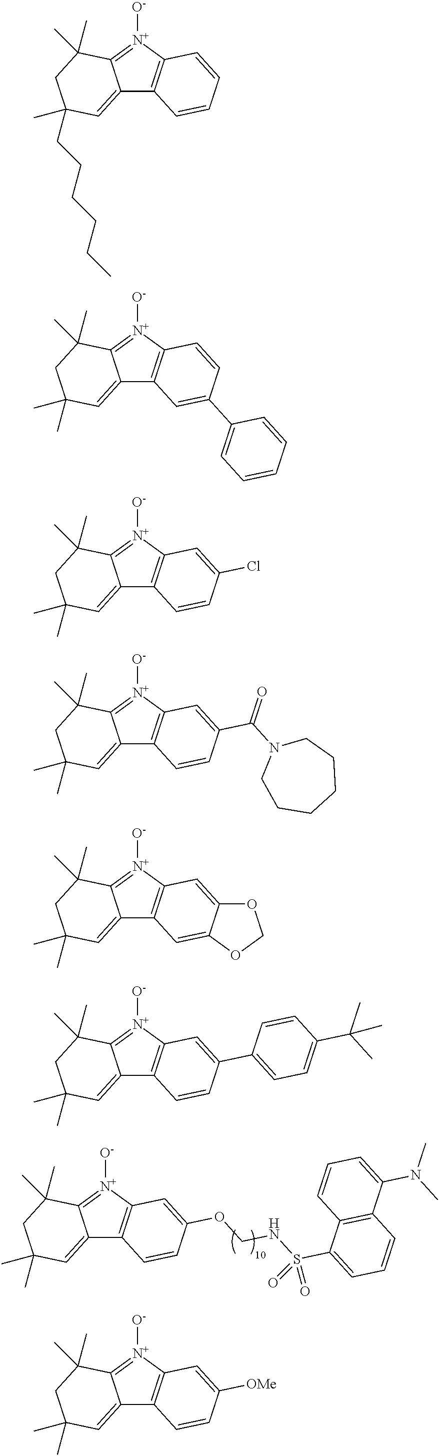

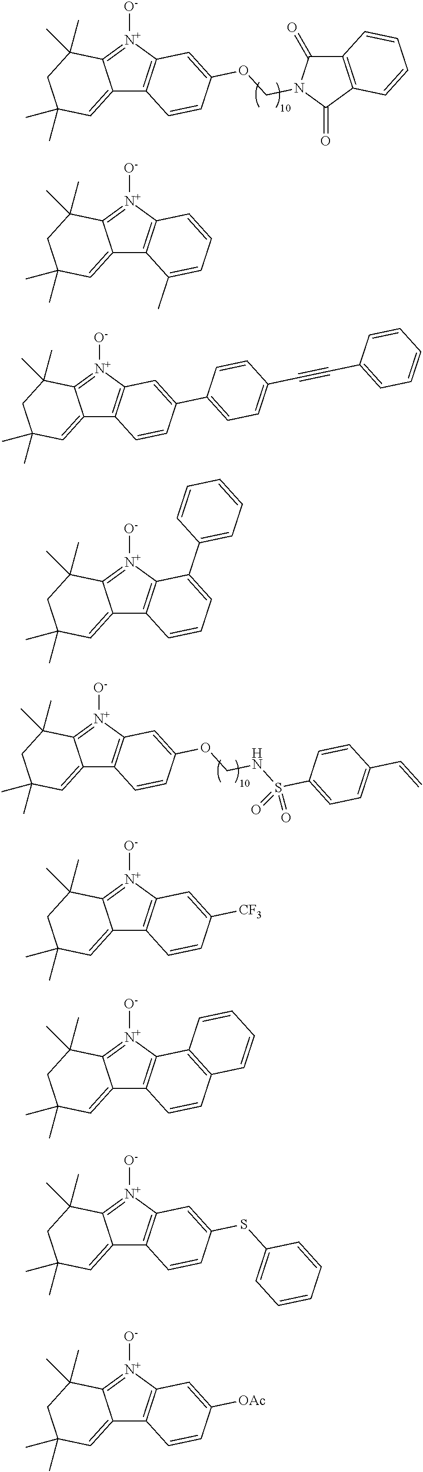

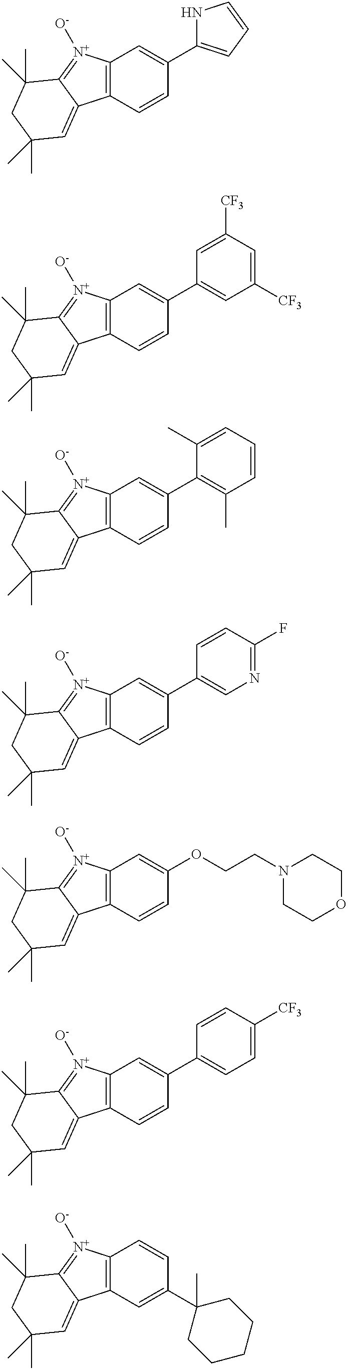

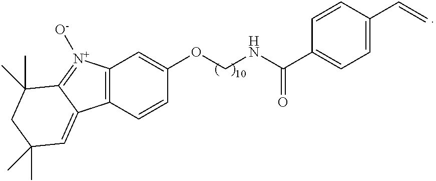

Exemplary compounds of the invention include a compound of formula:

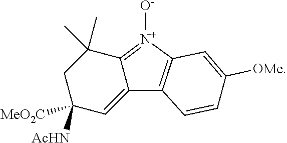

Exemplary compounds of the invention include a compound of formula:

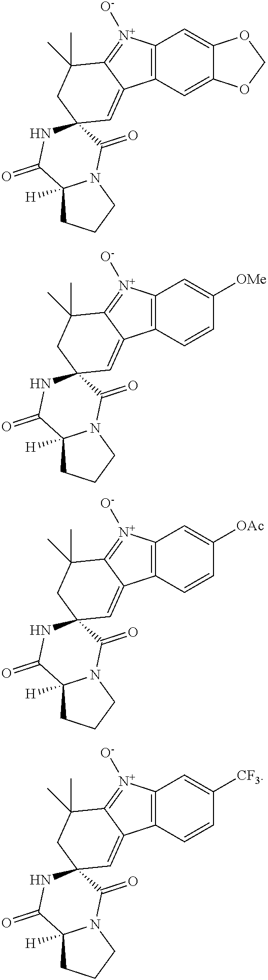

In certain embodiments, a nucleophile such as a thiol or alcohol is added to the α,β-unsaturated nitrone group of an inventive compound by a 1,5-addition to yield a compound of formula:

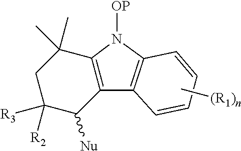

wherein

n, R₁, R₆, and R₇ are defined as described herein;

P is hydrogen or an oxygen-protecting group; and

Nu is hydrogen, —OR_{Nu}, —SR_{Nu}, —C(R_{Nu})₃, or —N(R_{Nu})₂, wherein each occurrence of R_{Nu} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety. In certain embodiments, the compound is of formula:

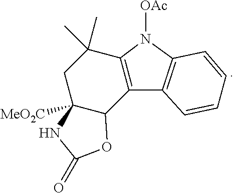

In certain embodiments, the compound is of formula:

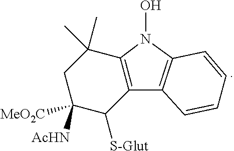

In certain embodiments, the compound is of formula:

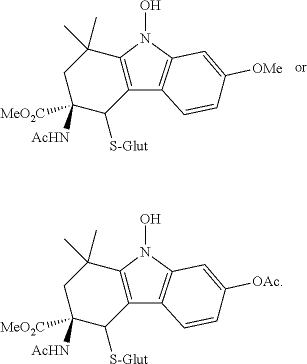

In certain embodiments, the compound is of formula:

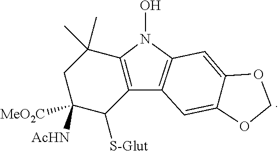

In certain embodiments, the present invention provides compounds of the formula:

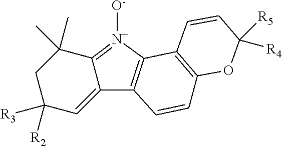

wherein

R₂, R₃, R₄, and R₅ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts, isomers, stereoisomers, enantiomers, diastereomers, and tautomers thereof.

In certain embodiments, R₂ is hydrogen. In certain embodiments, R₂ is substituted or unsubstituted aliphatic. In certain embodiments, R₂ is substituted or unsubstituted heteroaliphatic. In certain embodiments, R₂ is substituted or unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is —CO₂Me. In certain embodiments, R₂ is amino. In certain embodiments, R₂ is protected amino. In certain embodiments, R₂ is —NHAc. In certain embodiments, R₂ is alkylamino. In certain embodiments, R₂ is dialkylamino.

In certain embodiments, R₃ is hydrogen. In certain embodiments, R₃ is substituted or unsubstituted aliphatic. In certain embodiments, R₃ is substituted or unsubstituted heteroaliphatic. In certain embodiments, R₃ is substituted or unsubstituted alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is acyl. In certain embodiments, R₃ is —CO₂Me. In certain embodiments, R₃ is amino. In certain embodiments, R₃ is protected amino. In certain embodiments, R₃ is —NHAc. In certain embodiments, R₃ is alkylamino. In certain embodiments, R₃ is dialkylamino.

In certain embodiments, both R₂ and R₃ are hydrogen or C₁-C₆ alkyl. In certain embodiments, both R₂ and R₃ are hydrogen or methyl. In certain embodiments, both R₂ and R₃ are hydrogen. In certain embodiments, both R₂ and R₃ are C₁-C₆ alkyl. In certain embodiments, both R₂ and R₃ are methyl. In certain embodiments, both R₂ and R₃ are not methyl. In certain embodiments, R₂ and R₃ are taken together to form a cyclic structure.

In certain embodiments, R₄ is hydrogen. In certain embodiments, R₄ is substituted or unsubstituted aliphatic. In certain embodiments, R₄ is substituted or unsubstituted heteroaliphatic. In certain embodiments, R₄ is substituted or unsubstituted alkyl. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is acyl. In certain embodiments, R₄ is —CO₂Me. In certain embodiments, R₄ is amino. In certain embodiments, R₄ is protected amino. In certain embodiments, R₄ is —NHAc. In certain embodiments, R₄ is alkylamino. In certain embodiments, R₄ is dialkylamino.

In certain embodiments, R₅ is hydrogen. In certain embodiments, R₅ is substituted or unsubstituted aliphatic. In certain embodiments, R₅ is substituted or unsubstituted heteroaliphatic. In certain embodiments, R₅ is substituted or unsubstituted alkyl. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is acyl. In certain embodiments, R₅ is —CO₂Me. In certain embodiments, R₅ is amino. In certain embodiments, R₅ is protected amino. In certain embodiments, R₅ is —NHAc. In certain embodiments, R₅ is alkylamino. In certain embodiments, R₅ is dialkylamino.

In certain embodiments, both R₄ and R₅ are hydrogen or C₁-C₆ alkyl. In certain embodiments, both R₄ and R₅ are hydrogen or methyl. In certain embodiments, both R₄ and R₅ are hydrogen. In certain embodiments, both R₄ and R₅ are C₁-C₆ alkyl. In certain embodiments, both R₄ and R₅ are methyl. In certain embodiments, both R₄ and R₅ are not methyl. In certain embodiments, R₄ and R₅ are taken together to form a cyclic structure.

In certain embodiments, at least one of R₂, R₃, R₄, and R₅ is not methyl. In certain embodiments, at least two of R₂, R₃, R₄, and R₅ are not methyl. In certain embodiments, at least three of R₂, R₃, R₄, and R₅ is not methyl. In certain embodiments, at least one of R₂, R₃ is methyl, and at least one of R₄, and R₅ is methyl. In certain embodiments, only one of R₂, R₃ is methyl, and only one of R₄, and R₅ is methyl. In certain embodiments, at least one of R₂, R₃ is not methyl, and at least one of R₄, and R₅ is not methyl.

In certain embodiments, the compound is of formula:

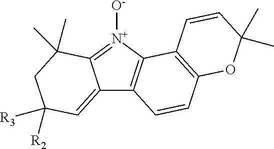

wherein R₂ and R₃ are defined as above.

In certain embodiments, the compounds is of formula:

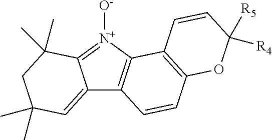

wherein R₄ and R₅ are defined as above.

In certain embodiments, the compounds is of formula:

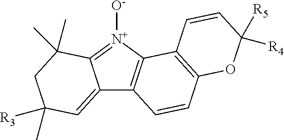

wherein R₃, R₄, and R₅ are defined as above.

In certain embodiments, the compounds is of formula:

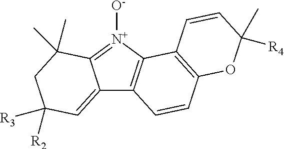

wherein R₂, R₃, and R₄ are defined as above.

In certain embodiments, the compounds is of formula:

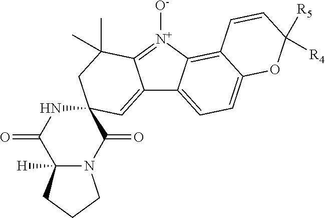

wherein R₄ and R₅ are defined as above. In certain embodiments, the compound is of the formula:

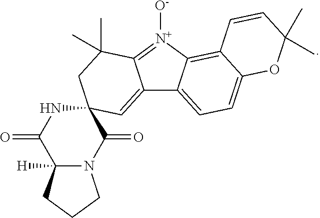

In certain embodiments, the compounds is of formula:

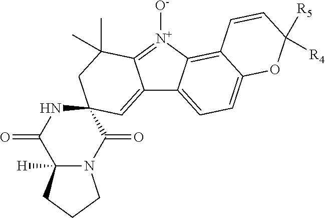

wherein R₄ and R₅ are defined as above. In certain embodiments, the compound is of the formula:

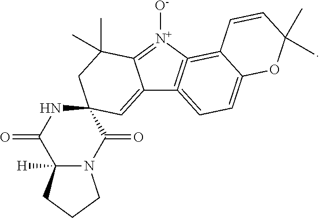

Exemplary compounds of the invention include compounds of formula:

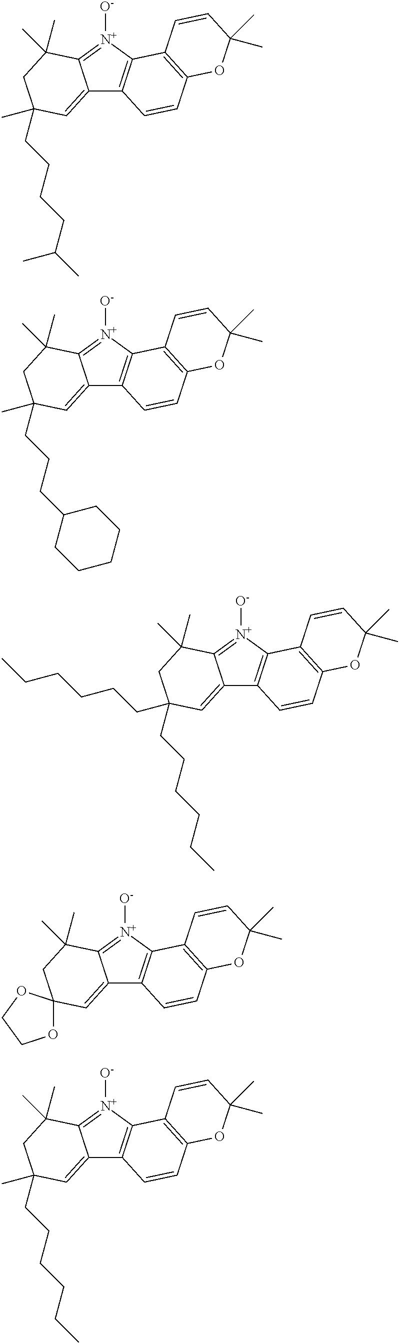

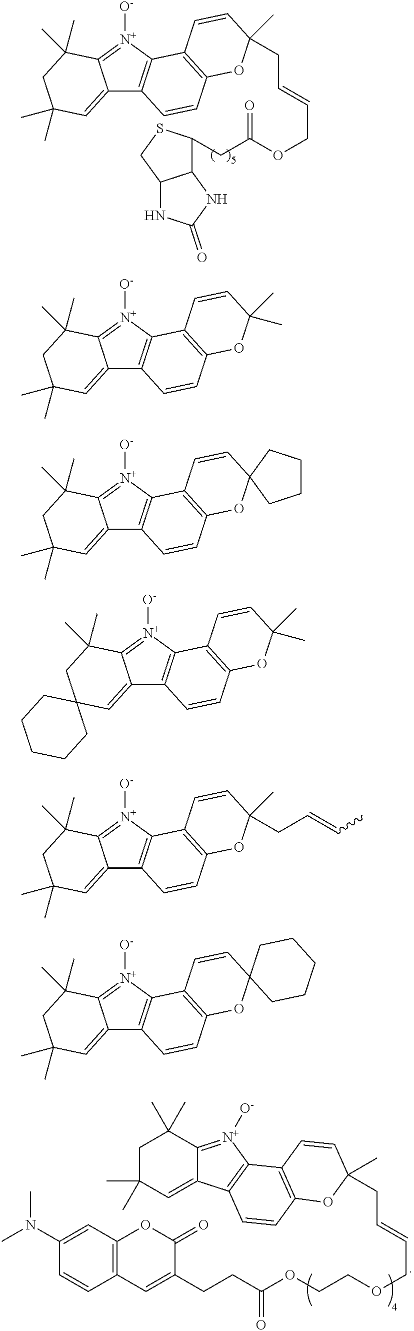

In certain embodiments, the present invention provides compounds of the formula:

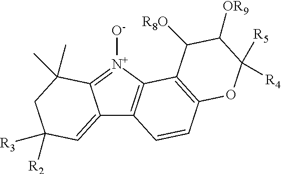

wherein

R₂, R₃, R₄, and R₅ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R₈ and R₉ are independently selected from the group consisting of hydrogen and C₁-C₆ alkyl; and pharmaceutically acceptable salts, isomers, stereoisomers, enantiomers, diastereomers, and tautomers thereof.

In certain embodiments, R₂ is hydrogen. In certain embodiments, R₂ is substituted or unsubstituted aliphatic. In certain embodiments, R₂ is substituted or unsubstituted heteroaliphatic. In certain embodiments, R₂ is substituted or unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is —CO₂Me. In certain embodiments, R₂ is amino. In certain embodiments, R₂ is protected amino. In certain embodiments, R₂ is —NHAc. In certain embodiments, R₂ is alkylamino. In certain embodiments, R₂ is dialkylamino.

In certain embodiments, R₃ is hydrogen. In certain embodiments, R₃ is substituted or unsubstituted aliphatic. In certain embodiments, R₃ is substituted or unsubstituted heteroaliphatic. In certain embodiments, R₃ is substituted or unsubstituted alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is acyl. In certain embodiments, R₃ is —CO₂Me. In certain embodiments, R₃ is amino. In certain embodiments, R₃ is protected amino. In certain embodiments, R₃ is —NHAc. In certain embodiments, R₃ is alkylamino. In certain embodiments, R₃ is dialkylamino.

In certain embodiments, both R₂ and R₃ are hydrogen or C₁-C₆ alkyl. In certain embodiments, both R₂ and R₃ are hydrogen or methyl. In certain embodiments, both R₂ and R₃ are hydrogen. In certain embodiments, both R₂ and R₃ are C₁-C₆ alkyl. In certain embodiments, both R₂ and R₃ are methyl. In certain embodiments, both R₂ and R₃ are not methyl. In certain embodiments, R₂ and R₃ are taken together to form a cyclic structure.

In certain embodiments, R₄ is hydrogen. In certain embodiments, R₄ is substituted or unsubstituted aliphatic. In certain embodiments, R₄ is substituted or unsubstituted heteroaliphatic. In certain embodiments, R₄ is substituted or unsubstituted alkyl. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is acyl. In certain embodiments, R₄ is —CO₂Me. In certain embodiments, R₄ is amino. In certain embodiments, R₄ is protected amino. In certain embodiments, R₄ is —NHAc. In certain embodiments, R₄ is alkylamino. In certain embodiments, R₄ is dialkylamino.

In certain embodiments, R₅ is hydrogen. In certain embodiments, R₅ is substituted or unsubstituted aliphatic. In certain embodiments, R₅ is substituted or unsubstituted heteroaliphatic. In certain embodiments, R₅ is substituted or unsubstituted alkyl. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is acyl. In certain embodiments, R₅ is —CO₂Me. In certain embodiments, R₅ is amino. In certain embodiments, R₅ is protected amino. In certain embodiments, R₅ is —NHAc. In certain embodiments, R₅ is alkylamino. In certain embodiments, R₅ is dialkylamino.

In certain embodiments, both R₄ and R₅ are hydrogen or C₁-C₆ alkyl. In certain embodiments, both R₄ and R₅ are hydrogen or methyl. In certain embodiments, both R₄ and R₅ are hydrogen. In certain embodiments, both R₄ and R₅ are C₁-C₆ alkyl. In certain embodiments, both R₄ and R₅ are methyl. In certain embodiments, both R₄ and R₅ are not methyl. In certain embodiments, R₄ and R₅ are taken together to form a cyclic structure.

In certain embodiments, at least one of R₂, R₃, R₄, and R₅ is not methyl. In certain embodiments, at least two of R₂, R₃, R₄, and R₅ are not methyl. In certain embodiments, at least three of R₂, R₃, R₄, and R₅ is not methyl. In certain embodiments, at least one of R₂, R₃ is methyl, and at least one of R₄, and R₅ is methyl. In certain embodiments, only one of R₂, R₃ is methyl, and only one of R₄, and R₅ is methyl. In certain embodiments, at least one of R₂, R₃ is not methyl, and at least one of R₄, and R₅ is not methyl.

In certain embodiments, R₈ is hydrogen. In certain embodiments, R₈ is C₁-C₆ alkyl. In certain embodiments, R₈ is methyl. In certain embodiments, R₈ is ethyl. In certain embodiments, R₈ is propyl.

In certain embodiments, R₉ is hydrogen. In certain embodiments, R₉ is C₁-C₆ alkyl. In certain embodiments, R₉ is methyl. In certain embodiments, R₉ is ethyl. In certain embodiments, R₉ is propyl.

In certain embodiments, both R₈ and R₉ are hydrogen. In certain embodiments, both R₈ and R₉ are C₁-C₆ alkyl. In certain embodiments, both R₈ and R₉ are hydrogen or methyl. In certain embodiments, both R₈ and R₉ are hydrogen. In certain embodiments, both R₈ and R₉ are C₁-C₆ alkyl. In certain embodiments, both R₈ and R₉ are methyl.

In certain embodiments, the compound is of formula:

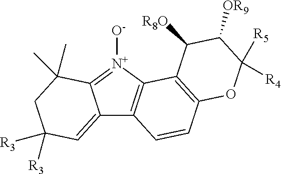

wherein R₂, R₃, R₄, R₅, R₈, and R₉ are defined as above.

In certain embodiments, the compound is of formula:

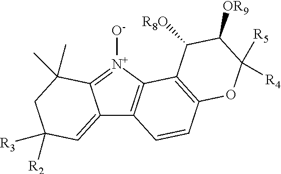

wherein R₂, R₃, R₄, R₅, R₈, and R₉ are defined as above.

Exemplary compounds of the invention include compounds of formula:

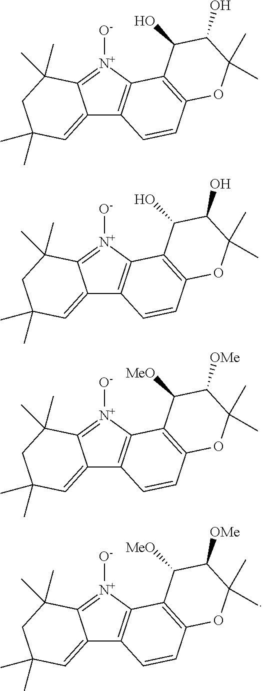

In certain embodiments, the present invention provides compounds of the formula:

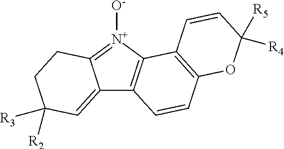

wherein

R₂, R₃, R₄, and R₅ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts, isomers, stereoisomers, enantiomers, diastereomers, and tautomers thereof.

In certain embodiments, R₂ is hydrogen. In certain embodiments, R₂ is substituted or unsubstituted aliphatic. In certain embodiments, R₂ is substituted or unsubstituted heteroaliphatic. In certain embodiments, R₂ is substituted or unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is —CO₂Me. In certain embodiments, R₂ is amino. In certain embodiments, R₂ is protected amino. In certain embodiments, R₂ is —NHAc. In certain embodiments, R₂ is alkylamino. In certain embodiments, R₂ is dialkylamino.

In certain embodiments, R₃ is hydrogen. In certain embodiments, R₃ is substituted or unsubstituted aliphatic. In certain embodiments, R₃ is substituted or unsubstituted heteroaliphatic. In certain embodiments, R₃ is substituted or unsubstituted alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is acyl. In certain embodiments, R₃ is —CO₂Me. In certain embodiments, R₃ is amino. In certain embodiments, R₃ is protected amino. In certain embodiments, R₃ is —NHAc. In certain embodiments, R₃ is alkylamino. In certain embodiments, R₃ is dialkylamino.

In certain embodiments, both R₂ and R₃ are hydrogen or C₁-C₆ alkyl. In certain embodiments, both R₂ and R₃ are hydrogen or methyl. In certain embodiments, both R₂ and R₃ are hydrogen. In certain embodiments, both R₂ and R₃ are C₁-C₆ alkyl. In certain embodiments, both R₂ and R₃ are methyl. In certain embodiments, both R₂ and R₃ are not methyl. In certain embodiments, R₂ and R₃ are taken together to form a cyclic structure.

In certain embodiments, R₄ is hydrogen. In certain embodiments, R₄ is substituted or unsubstituted aliphatic. In certain embodiments, R₄ is substituted or unsubstituted heteroaliphatic. In certain embodiments, R₄ is substituted or unsubstituted alkyl. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is acyl. In certain embodiments, R₄ is —CO₂Me. In certain embodiments, R₄ is amino. In certain embodiments, R₄ is protected amino. In certain embodiments, R₄ is —NHAc. In certain embodiments, R₄ is alkylamino. In certain embodiments, R₄ is dialkylamino.

In certain embodiments, R₅ is hydrogen. In certain embodiments, R₅ is substituted or unsubstituted aliphatic. In certain embodiments, R₅ is substituted or unsubstituted heteroaliphatic. In certain embodiments, R₅ is substituted or unsubstituted alkyl. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is acyl. In certain embodiments, R₅ is —CO₂Me. In certain embodiments, R₅ is amino. In certain embodiments, R₅ is protected amino. In certain embodiments, R₅ is —NHAc. In certain embodiments, R₅ is alkylamino. In certain embodiments, R₅ is dialkylamino.

In certain embodiments, both R₄ and R₅ are hydrogen or C₁-C₆ alkyl. In certain embodiments, both R₄ and R₅ are hydrogen or methyl. In certain embodiments, both R₄ and R₅ are hydrogen. In certain embodiments, both R₄ and R₅ are C₁-C₆ alkyl. In certain embodiments, both R₄ and R₅ are methyl. In certain embodiments, both R₄ and R₅ are not methyl. In certain embodiments, R₄ and R₅ are taken together to form a cyclic structure.

In certain embodiments, at least one of R₂, R₃, R₄, and R₅ is not methyl. In certain embodiments, at least two of R₂, R₃, R₄, and R₅ are not methyl. In certain embodiments, at least three of R₂, R₃, R₄, and R₅ is not methyl. In certain embodiments, at least one of R₂, R₃ is methyl, and at least one of R₄, and R₅ is methyl. In certain embodiments, only one of R₂, R₃ is methyl, and only one of R₄, and R₅ is methyl. In certain embodiments, at least one of R₂, R₃ is not methyl, and at least one of R₄, and R₅ is not methyl.

In certain embodiments, the compound is of formula:

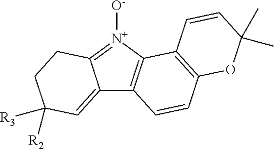

wherein R₂ and R₃ are defined as above.

In certain embodiments, the compounds is of formula:

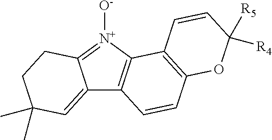

wherein R₄ and R₅ are defined as above.

In certain embodiments, the compounds is of formula:

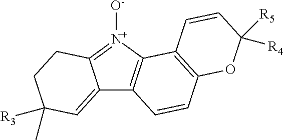

wherein R₃, R₄, and R₅ are defined as above.

In certain embodiments, the compounds is of formula:

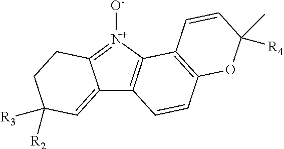

wherein R₂, R₃, and R₄ are defined as above.

In certain embodiments, the compounds is of formula:

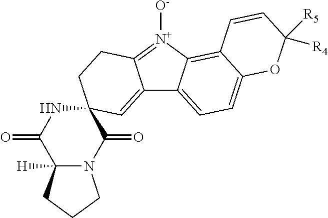

wherein R₄ and R₅ are defined as above. In certain embodiments, the compound is of the formula:

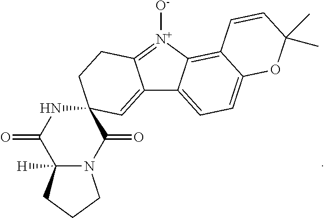

In certain embodiments, the compounds is of formula:

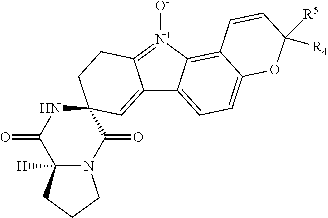

wherein R₄ and R₅ are defined as above. In certain embodiments, the compound is of the formula:

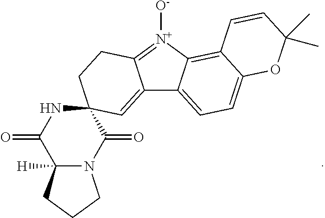

Exemplary compounds of the invention include compounds of formula:

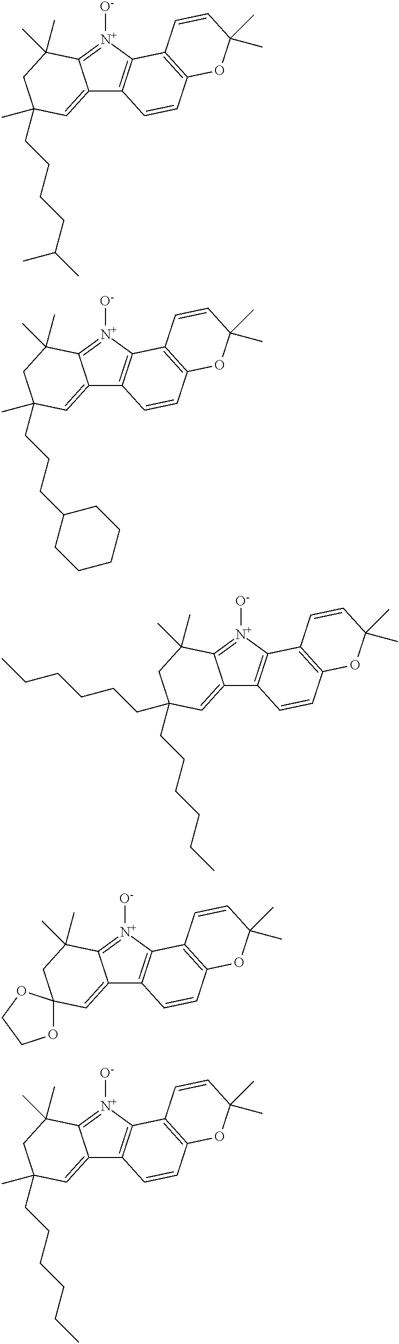

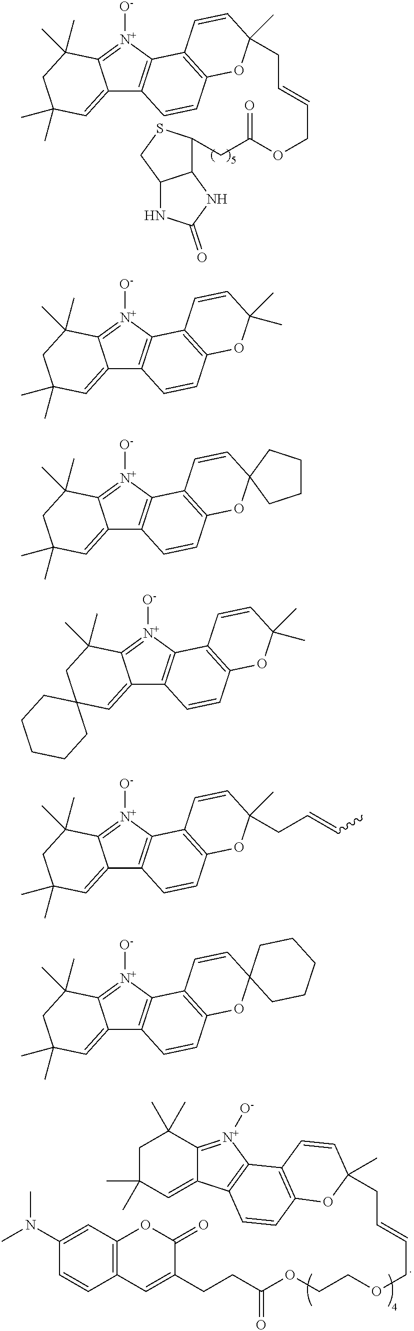

In certain embodiments, the present invention provides compounds of the formula:

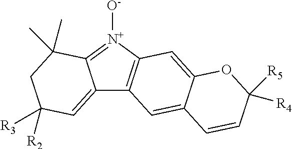

wherein

R₂, R₃, R₄, and R₅ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts, isomers, stereoisomers, enantiomers, diastereomers, and tautomers thereof.

In certain embodiments, R₂ is hydrogen. In certain embodiments, R₂ is substituted or unsubstituted aliphatic. In certain embodiments, R₂ is substituted or unsubstituted heteroaliphatic. In certain embodiments, R₂ is substituted or unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is —CO₂Me. In certain embodiments, R₂ is amino. In certain embodiments, R₂ is protected amino. In certain embodiments, R₂ is —NHAc. In certain embodiments, R₂ is alkylamino. In certain embodiments, R₂ is dialkylamino.

In certain embodiments, R₃ is hydrogen. In certain embodiments, R₃ is substituted or unsubstituted aliphatic. In certain embodiments, R₃ is substituted or unsubstituted heteroaliphatic. In certain embodiments, R₃ is substituted or unsubstituted alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is acyl. In certain embodiments, R₃ is —CO₂Me. In certain embodiments, R₃ is amino. In certain embodiments, R₃ is protected amino. In certain embodiments, R₃ is —NHAc. In certain embodiments, R₃ is alkylamino. In certain embodiments, R₃ is dialkylamino.

In certain embodiments, both R₂ and R₃ are hydrogen or C₁-C₆ alkyl. In certain embodiments, both R₂ and R₃ are hydrogen or methyl. In certain embodiments, both R₂ and R₃ are hydrogen. In certain embodiments, both R₂ and R₃ are C₁-C₆ alkyl. In certain embodiments, both R₂ and R₃ are methyl. In certain embodiments, both R₂ and R₃ are not methyl. In certain embodiments, R₂ and R₃ are taken together to form a cyclic structure.

In certain embodiments, R₄ is hydrogen. In certain embodiments, R₄ is substituted or unsubstituted aliphatic. In certain embodiments, R₄ is substituted or unsubstituted heteroaliphatic. In certain embodiments, R₄ is substituted or unsubstituted alkyl. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is acyl. In certain embodiments, R₄ is —CO₂Me. In certain embodiments, R₄ is amino. In certain embodiments, R₄ is protected amino. In certain embodiments, R₄ is —NHAc. In certain embodiments, R₄ is alkylamino. In certain embodiments, R₄ is dialkylamino.

In certain embodiments, R₅ is hydrogen. In certain embodiments, R₅ is substituted or unsubstituted aliphatic. In certain embodiments, R₅ is substituted or unsubstituted heteroaliphatic. In certain embodiments, R₅ is substituted or unsubstituted alkyl. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is acyl. In certain embodiments, R₅ is —CO₂Me. In certain embodiments, R₅ is amino. In certain embodiments, R₅ is protected amino. In certain embodiments, R₅ is —NHAc. In certain embodiments, R₅ is alkylamino. In certain embodiments, R₅ is dialkylamino.

In certain embodiments, both R₄ and R₅ are hydrogen or C₁-C₆ alkyl. In certain embodiments, both R₄ and R₅ are hydrogen or methyl. In certain embodiments, both R₄ and R₅ are hydrogen. In certain embodiments, both R₄ and R₅ are C₁-C₆ alkyl. In certain embodiments, both R₄ and R₅ are methyl. In certain embodiments, both R₄ and R₅ are not methyl. In certain embodiments, R₄ and R₅ are taken together to form a cyclic structure.

In certain embodiments, at least one of R₂, R₃, R₄, and R₅ is not methyl. In certain embodiments, at least two of R₂, R₃, R₄, and R₅ are not methyl. In certain embodiments, at least three of R₂, R₃, R₄, and R₅ is not methyl. In certain embodiments, at least one of R₂, R₃ is methyl, and at least one of R₄, and R₅ is methyl. In certain embodiments, only one of R₂, R₃ is methyl, and only one of R₄, and R₅ is methyl. In certain embodiments, at least one of R₂, R₃ is not methyl, and at least one of R₄, and R₅ is not methyl.

In certain embodiments, the compound is of formula:

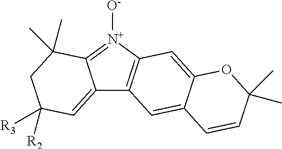

wherein R₂ and R₃ are defined as above.

In certain embodiments, the compounds is of formula:

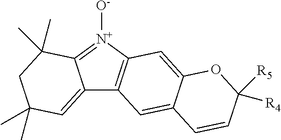

wherein R₄ and R₅ are defined as above.

In certain embodiments, the compounds is of formula:

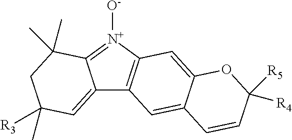

wherein R₃, R₄, and R₅ are defined as above.

In certain embodiments, the compounds is of formula:

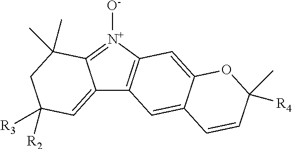

wherein R₂, R₃, and R₄ are defined as above.

Exemplary compounds of the invention include compounds of formula:

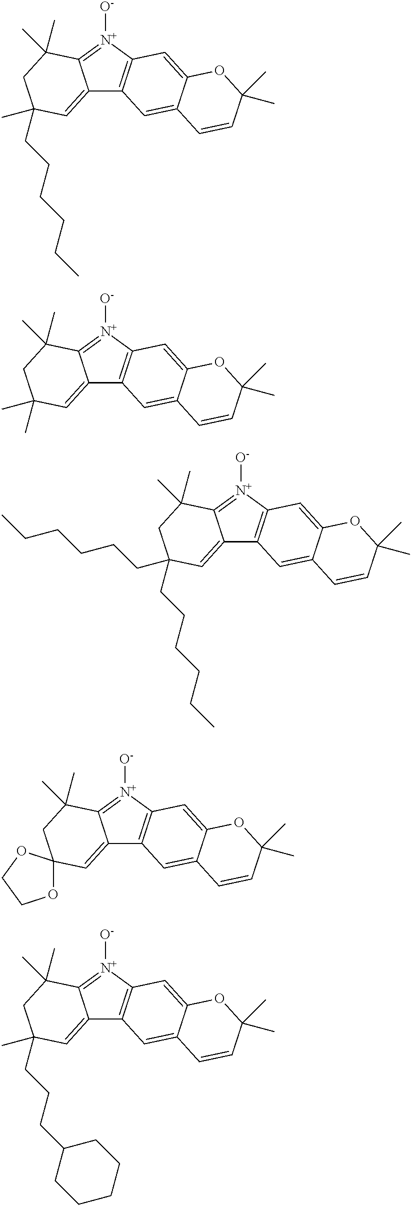

In certain embodiments, the present invention provides compounds of the formula:

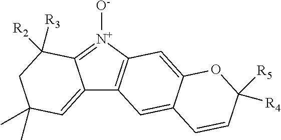

wherein

R₂, R₃, R₄, and R₅ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts, isomers, stereoisomers, enantiomers, diastereomers, and tautomers thereof.

In certain embodiments, R₂ is hydrogen. In certain embodiments, R₂ is substituted or unsubstituted aliphatic. In certain embodiments, R₂ is substituted or unsubstituted heteroaliphatic. In certain embodiments, R₂ is substituted or unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is —CO₂Me. In certain embodiments, R₂ is amino. In certain embodiments, R₂ is protected amino. In certain embodiments, R₂ is —NHAc. In certain embodiments, R₂ is alkylamino. In certain embodiments, R₂ is dialkylamino.

In certain embodiments, R₃ is hydrogen. In certain embodiments, R₃ is substituted or unsubstituted aliphatic. In certain embodiments, R₃ is substituted or unsubstituted heteroaliphatic. In certain embodiments, R₃ is substituted or unsubstituted alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is acyl. In certain embodiments, R₃ is —CO₂Me. In certain embodiments, R₃ is amino. In certain embodiments, R₃ is protected amino. In certain embodiments, R₃ is —NHAc. In certain embodiments, R₃ is alkylamino. In certain embodiments, R₃ is dialkylamino.

In certain embodiments, both R₂ and R₃ are hydrogen or C₁-C₆ alkyl. In certain embodiments, both R₂ and R₃ are hydrogen or methyl. In certain embodiments, both R₂ and R₃ are hydrogen. In certain embodiments, both R₂ and R₃ are C₁-C₆ alkyl. In certain embodiments, both R₂ and R₃ are methyl. In certain embodiments, both R₂ and R₃ are not methyl. In certain embodiments, R₂ and R₃ are taken together to form a cyclic structure.

In certain embodiments, R₄ is hydrogen. In certain embodiments, R₄ is substituted or unsubstituted aliphatic. In certain embodiments, R₄ is substituted or unsubstituted heteroaliphatic. In certain embodiments, R₄ is substituted or unsubstituted alkyl. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is acyl. In certain embodiments, R₄ is —CO₂Me. In certain embodiments, R₄ is amino. In certain embodiments, R₄ is protected amino. In certain embodiments, R₄ is —NHAc. In certain embodiments, R₄ is alkylamino. In certain embodiments, R₄ is dialkylamino.

In certain embodiments, R₅ is hydrogen. In certain embodiments, R₅ is substituted or unsubstituted aliphatic. In certain embodiments, R₅ is substituted or unsubstituted heteroaliphatic. In certain embodiments, R₅ is substituted or unsubstituted alkyl. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is acyl. In certain embodiments, R₅ is —CO₂Me. In certain embodiments, R₅ is amino. In certain embodiments, R₅ is protected amino. In certain embodiments, R₅ is —NHAc. In certain embodiments, R₅ is alkylamino. In certain embodiments, R₅ is dialkylamino.

In certain embodiments, both R₄ and R₅ are hydrogen or C₁-C₆ alkyl. In certain embodiments, both R₄ and R₅ are hydrogen or methyl. In certain embodiments, both R₄ and R₅ are hydrogen. In certain embodiments, both R₄ and R₅ are C₁-C₆ alkyl. In certain embodiments, both R₄ and R₅ are methyl. In certain embodiments, both R₄ and R₅ are not methyl. In certain embodiments, R₄ and R₅ are taken together to form a cyclic structure.

In certain embodiments, at least one of R₂, R₃, R₄, and R₅ is not methyl. In certain embodiments, at least two of R₂, R₃, R₄, and R₅ are not methyl. In certain embodiments, at least three of R₂, R₃, R₄, and R₅ is not methyl. In certain embodiments, at least one of R₂, R₃ is methyl, and at least one of R₄, and R₅ is methyl. In certain embodiments, only one of R₂, R₃ is methyl, and only one of R₄, and R₅ is methyl. In certain embodiments, at least one of R₂, R₃ is not methyl, and at least one of R₄, and R₅ is not methyl.

In certain embodiments, the compound is of formula:

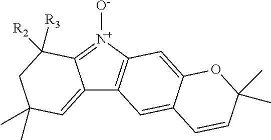

wherein R₂ and R₃ are defined as above.

In certain embodiments, the compounds is of formula:

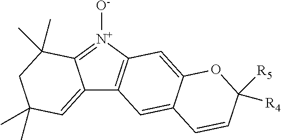

wherein R₄ and R₅ are defined as above.

In certain embodiments, the compounds is of formula:

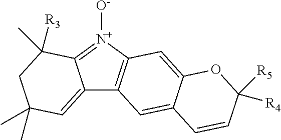

wherein R₃, R₄, and R₅ are defined as above.

In certain embodiments, the compounds is of formula:

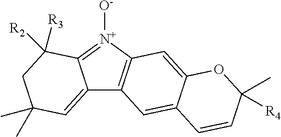

wherein R₂, R₃, and R₄ are defined as above.

Exemplary compounds of the invention include compounds of formula:

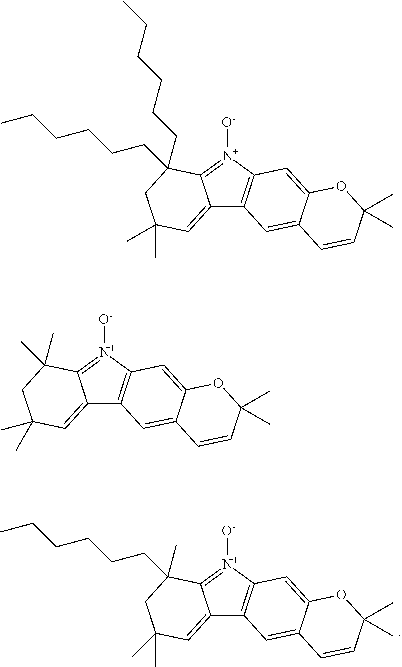

In certain embodiments, the present invention provides compounds of the formula:

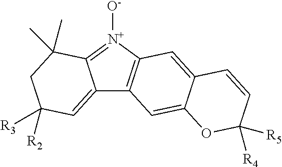

wherein

R₂, R₃, R₄, and R₅ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and pharmaceutically acceptable salts, isomers, stereoisomers, enantiomers, diastereomers, and tautomers thereof.

In certain embodiments, R₂ is hydrogen. In certain embodiments, R₂ is substituted or unsubstituted aliphatic. In certain embodiments, R₂ is substituted or unsubstituted heteroaliphatic. In certain embodiments, R₂ is substituted or unsubstituted alkyl. In certain embodiments, R₂ is C₁-C₆ alkyl. In certain embodiments, R₂ is methyl. In certain embodiments, R₂ is ethyl. In certain embodiments, R₂ is propyl. In certain embodiments, R₂ is acyl. In certain embodiments, R₂ is —CO₂Me. In certain embodiments, R₂ is amino. In certain embodiments, R₂ is protected amino. In certain embodiments, R₂ is —NHAc. In certain embodiments, R₂ is alkylamino. In certain embodiments, R₂ is dialkylamino.

In certain embodiments, R₃ is hydrogen. In certain embodiments, R₃ is substituted or unsubstituted aliphatic. In certain embodiments, R₃ is substituted or unsubstituted heteroaliphatic. In certain embodiments, R₃ is substituted or unsubstituted alkyl. In certain embodiments, R₃ is C₁-C₆ alkyl. In certain embodiments, R₃ is methyl. In certain embodiments, R₃ is ethyl. In certain embodiments, R₃ is propyl. In certain embodiments, R₃ is acyl. In certain embodiments, R₃ is —CO₂Me. In certain embodiments, R₃ is amino. In certain embodiments, R₃ is protected amino. In certain embodiments, R₃ is —NHAc. In certain embodiments, R₃ is alkylamino. In certain embodiments, R₃ is dialkylamino.

In certain embodiments, both R₂ and R₃ are hydrogen or C₁-C₆ alkyl. In certain embodiments, both R₂ and R₃ are hydrogen or methyl. In certain embodiments, both R₂ and R₃ are hydrogen. In certain embodiments, both R₂ and R₃ are C₁-C₆ alkyl. In certain embodiments, both R₂ and R₃ are methyl. In certain embodiments, both R₂ and R₃ are not methyl. In certain embodiments, R₂ and R₃ are taken together to form a cyclic structure.

In certain embodiments, R₄ is hydrogen. In certain embodiments, R₄ is substituted or unsubstituted aliphatic. In certain embodiments, R₄ is substituted or unsubstituted heteroaliphatic. In certain embodiments, R₄ is substituted or unsubstituted alkyl. In certain embodiments, R₄ is C₁-C₆ alkyl. In certain embodiments, R₄ is methyl. In certain embodiments, R₄ is ethyl. In certain embodiments, R₄ is propyl. In certain embodiments, R₄ is acyl. In certain embodiments, R₄ is —CO₂Me. In certain embodiments, R₄ is amino. In certain embodiments, R₄ is protected amino. In certain embodiments, R₄ is —NHAc. In certain embodiments, R₄ is alkylamino. In certain embodiments, R₄ is dialkylamino.

In certain embodiments, R₅ is hydrogen. In certain embodiments, R₅ is substituted or unsubstituted aliphatic. In certain embodiments, R₅ is substituted or unsubstituted heteroaliphatic. In certain embodiments, R₅ is substituted or unsubstituted alkyl. In certain embodiments, R₅ is C₁-C₆ alkyl. In certain embodiments, R₅ is methyl. In certain embodiments, R₅ is ethyl. In certain embodiments, R₅ is propyl. In certain embodiments, R₅ is acyl. In certain embodiments, R₅ is —CO₂Me. In certain embodiments, R₅ is amino. In certain embodiments, R₅ is protected amino. In certain embodiments, R₅ is —NHAc. In certain embodiments, R₅ is alkylamino. In certain embodiments, R₅ is dialkylamino.

In certain embodiments, both R₄ and R₅ are hydrogen or C₁-C₆ alkyl. In certain embodiments, both R₄ and R₅ are hydrogen or methyl. In certain embodiments, both R₄ and R₅ are hydrogen. In certain embodiments, both R₄ and R₅ are C₁-C₆ alkyl. In certain embodiments, both R₄ and R₅ are methyl. In certain embodiments, both R₄ and R₅ are not methyl. In certain embodiments, R₄ and R₅ are taken together to form a cyclic structure.

In certain embodiments, at least one of R₂, R₃, R₄, and R₅ is not methyl. In certain embodiments, at least two of R₂, R₃, R₄, and R₅ are not methyl. In certain embodiments, at least three of R₂, R₃, R₄, and R₅ is not methyl. In certain embodiments, at least one of R₂, R₃ is methyl, and at least one of R₄, and R₅ is methyl. In certain embodiments, only one of R₂, R₃ is methyl, and only one of R₄, and R₅ is methyl. In certain embodiments, at least one of R₂, R₃ is not methyl, and at least one of R₄, and R₅ is not methyl.

In certain embodiments, the compound is of formula:

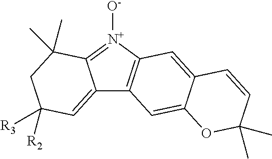

wherein R₂ and R₃ are defined as above.

In certain embodiments, the compounds is of formula:

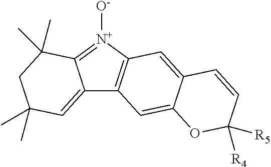

wherein R₄ and R₅ are defined as above.

In certain embodiments, the compounds is of formula:

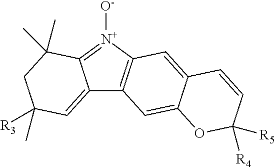

wherein R₃, R₄, and R₅ are defined as above.

In certain embodiments, the compounds is of formula:

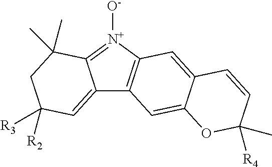

wherein R₂, R₃, and R₄ are defined as above.

Exemplary compounds of the invention include compounds of formula:

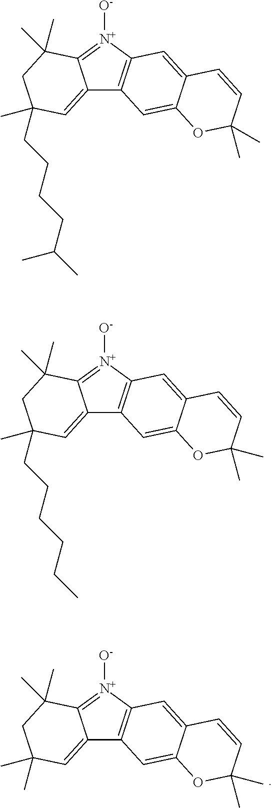

In certain embodiments, the inventive avrainvillamide analogue is tagged with a detectable label. In certain embodiments, the analogue is tagged with biotin. In certain embodiments, the analogue is tagged with a fluorescent label. In certain embodiments, the analogue is tagged with a dansyl moiety. In certain embodiments, the biotin labeled analogue is of formula:

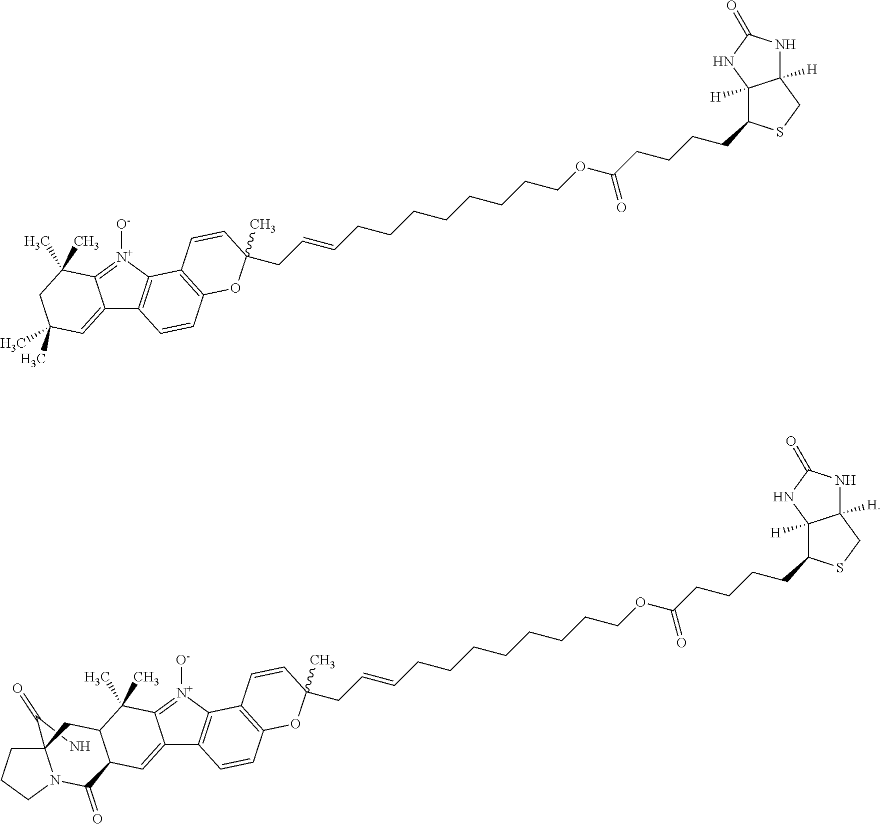

In certain embodiments, the dansylated analogue is of formula:

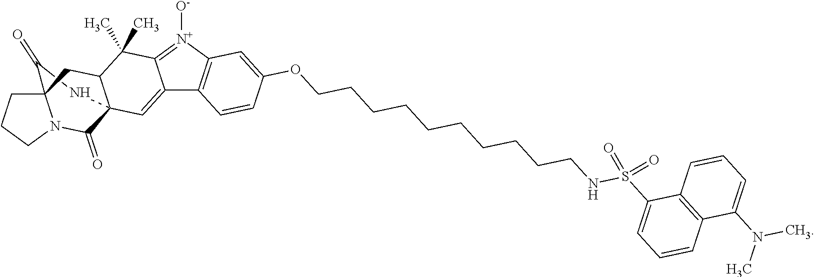

### Methods of Synthesis

A synthesis of avrainvillamide and analogues thereof is described in published PCT application, WO 2006/102097, published Sep. 28, 2006, which is incorporated herein by reference. As would be appreciated by one of skill in the art, the synthetic methodology described in WO 2006/102097 can also be applied to the compounds of the present application. Exemplary syntheses of particular compounds of the invention are described in the Examples below.

An exemplary synthesis of avrainvillamide is also shown in the scheme below. As will be appreciated by one of skill in this art, various modification can be made to the starting materials and reagents used in the scheme to provide the compounds of the invention.

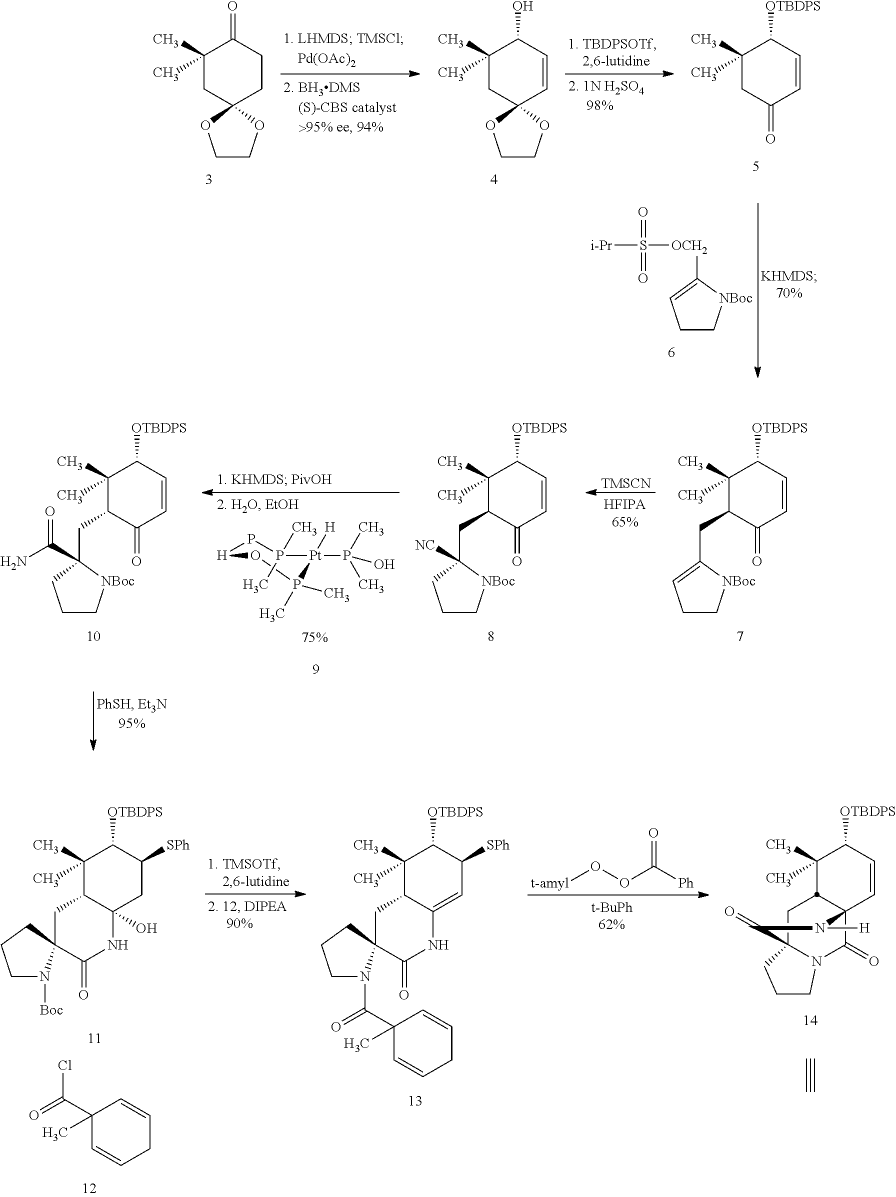

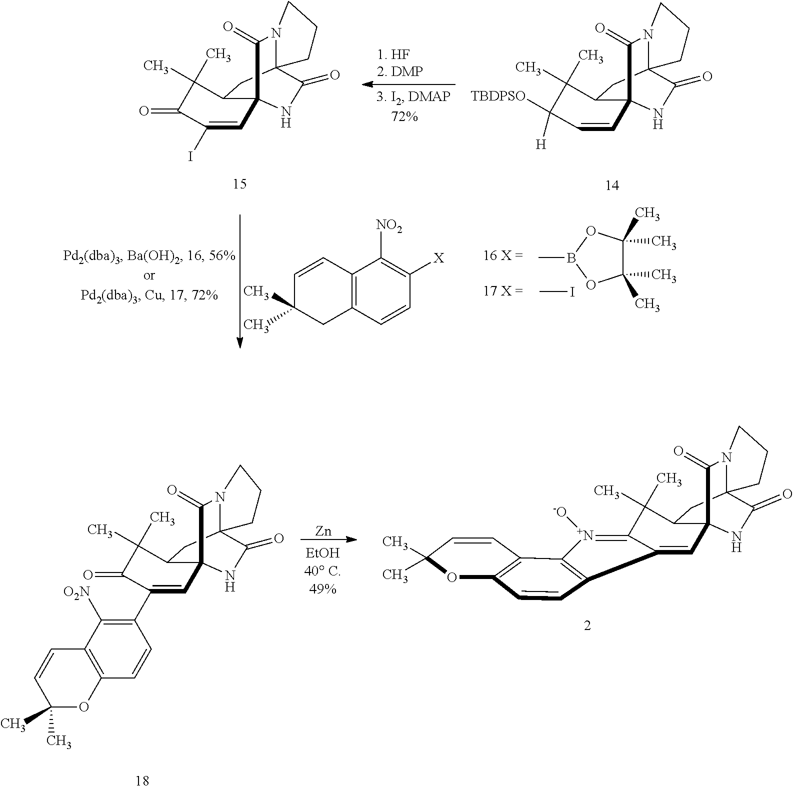

The synthesis of avrainvillamide begins with the achiral cyclohexanone derivative 3; however, other chiral or achiral cyclohexanone derivatives may also be used as the starting material. The cyclohexanone derivative is transformed via its protected enol ether into the corresponding α,β-unsaturated ketone. This oxidation reaction can be accomplished by palladium-mediated oxidation as shown. Other oxidation methods which may be used include the oxidation with 2-iodoxybenzoic acid in the presence of 4-methoxypyridine N-oxide. As will be appreciated by one of skill in this art, other oxidation may also be used to effect this transformation.

The resulting α,β-unsaturated ketone is reduced enantioselectively. In one embodiment, the Corey-Bakshi-Shibata catalyst is used in the reduction. Either the (S)-CBS catalyst or the (R)-CBS catalyst may be used in the reduction reaction to achieve either enantiomer. The (S)-CBS catalyst was used for the (R)-allylic alcohol. In other embodiments, another enantioselective catalyst is utilized. In certain embodiments, the α,β-unsaturated ketone is reduced to give a mixture of enantiomers or diastereomers, and the desired isomer is purified. In the synthesis shown above, the stereochemistry introduced by the CBS reduction is subsequently relayed to all other stereocenters in avrainvillamide and stephacidin B.

The resulting allylic alcohol is protected (e.g., as the silyl ether), and the ketal group is hydrolysed to yield the α,β-unsaturated ketone 5. The ketone 5 is deprotonated at the α-position using a base (e.g., potassium hexamethyldisilazide (KHMDS), LDA), and the resulting enolate is reacted with electrophile 6, which can be prepared from N-(tert-butoxycarbonyl)-2,3-dihydropyrrole by a sequence involving α-lithiation, formylation, reduction (e.g., borohydride), and iso-propylsulfonylation. The resulting trans-coupling product 7 is formed as a single diastereomer. The alkylation product 7 underwent Strecker-like addition of hydrogen cyanide in hexyluoroisopropanol (HFIPA) forming the N-Boc amino nitrile 8. To establish the stereorelationships required for the synthesis of stephacidin B, the α-carbon of the ketone 8 was epimerized (e.g., by deprotonation with base (e.g., KHDMS) followed by quenching with pivalic acid). The platinum catalyst 9 was then used to transform the nitrile group of the epimerized product into the corresponding primary amide. Treatment of the resulting primary amide 10 with thiophenol and triethylamine led to conjugate addition of thiophenol as well as cyclic hemiaminal formation, giving the tricyclic product 11. Dehydration of the cyclic hemiaminal 11 in the presence of trimethylsilyl triflate and 2,6-lutidine was accompanied by cleavage of the N-Boc protective group. Amide 13 was then formed by the acylation of the pyrrolidinyl amine group that was liberated with 1-methyl-2,5-cyclohexadiene-1-carbonyl chloride. Heating of rigorously deoxygenated solutions of 13 and t-amyl peroxybenzoate in t-butyl benzene as solvent produced the bridged diketopiperazine core of avrainvillamide.

The tetracyclic product 14 was then transformed into the α-iodoenone 15 in a three-step sequence as shown. The α-iodoenone 15 was coupled in a Suzuki reaction with the arylboronic acid derivative 16 or by Ullmann-like coupling with the aryl iodide 17. The nitroarene coupling product was reduced in the presence of activated zinc powder, forming the heptacyclic unsaturated nitrone 2.

### Pharmaceutical Compositions

This invention also provides a pharmaceutical preparation comprising at least one of the compounds as described above and herein, or a pharmaceutically acceptable derivative thereof, which compounds inhibit the growth of or kill tumor cells. In other embodiments, the compounds show cytostatic or cytotoxic activity against neoplastic cells such as cancer cells. In yet other embodiments, the compounds inhibit the growth of or kill rapidly dividing cells such as stimulated inflammatory cells. In certain other embodiments, the compounds have anti-microbial activity.

As discussed above, the present invention provides novel compounds having anti-microbial and/or anti-proliferative activity, and thus the inventive compounds are useful for the treatment of a variety of medical conditions including infectious diseases, cancer, autoimmune diseases, inflammatory diseases, and diabetic retinopathy. Accordingly, in another aspect of the present invention, pharmaceutical compositions are provided, wherein these compositions comprise any one of the compounds as described herein, and optionally comprise a pharmaceutically acceptable carrier. In certain embodiments, these compositions optionally further comprise one or more additional therapeutic agents, e.g., another anti-microbial agent or another anti-proliferative agent. In other embodiments, these compositions further comprise an anti-inflammatory agent such as aspirin, ibuprofen, acetaminophen, etc., pain reliever, or anti-pyretic. In other embodiments, these compositions further comprise an anti-emetic agent, a pain reliever, a multi-vitamin, etc.

It will also be appreciated that certain of the compounds of the present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or any other adduct or derivative which upon administration to a patient in need is capable of providing, directly or indirectly, a compound as otherwise described herein, or a metabolite or residue thereof, e.g., a prodrug.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19, 1977; incorporated herein by reference. The salts can be prepared in situ during the final isolation and purification of the compounds of the invention, or separately by reacting the free base functionality with a suitable organic or inorganic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates. In certain embodiments, the esters are cleaved by enzymes such as esterases.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicles, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-cancer compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; Cremophor; Solutol; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Uses of Compounds and Pharmaceutical Compositions

The invention further provides a method of treating infections and inhibiting tumor growth. The method involves the administration of a therapeutically effective amount of the compound or a pharmaceutically acceptable derivative thereof to a subject (including, but not limited to a human or animal) in need of it.

The compounds and pharmaceutical compositions of the present invention may be used in treating or preventing any disease or conditions including infections (e.g., skin infections, GI infection, urinary tract infections, genito-urinary infections, systemic infections), proliferative diseases (e.g., cancer, benign neoplasms, diabetic retinopathy), and autoimmune diseases (e.g., rheumatoid arthritis, lupus). The compounds and pharmaceutical compositions may be administered to animals, preferably mammals (e.g., domesticated animals, cats, dogs, mice, rats), and more preferably humans. Any method of administration may be used to deliver the compound of pharmaceutical compositions to the animal. In certain embodiments, the compound or pharmaceutical composition is administered orally. In other embodiments, the compound or pharmaceutical composition is administered parenterally.

The exact amount required will vary from subject to subject, depending on the species, age, and general condition of the subject, the particular compound, its mode of administration, its mode of activity, and the like. The compounds of the invention are preferably formulated in dosage unit form for ease of administration and uniformity of dosage. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

Liquid dosage forms for oral and parenteral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents. In certain embodiments for parenteral administration, the compounds of the invention are mixed with solubilizing agents such an Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

In still another aspect, the present invention also provides a pharmaceutical pack or kit comprising one or more containers filled with one or more of the ingredients of the pharmaceutical compositions of the invention, and in certain embodiments, includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use or sale for human administration.

### Biological Target

Nucleophosmin (NPM1.1, B23, numatrin, NO38) has been identified as a principle target for avrainvillamide and analogues by affinity isolation, MS sequencing, and Western blot. A synthetic biotin-avrainvillamide conjugate (described below in the Examples), which was nearly equipotent to (+)-avrainvillamide in inhibiting the growth of T-47D cells, was used for affinity-isolation of a protein identified as nucleophosmin by MS sequencing and Western blotting. The binding of the biotin-avrainvillamide conjugate was inhibited by iodoacetamide, (+)-avrainvillamide, and various structural analogues of (+)-avrainvillamide.

Identification of nucleophosmin as a target of avrainvillamide allows for the screening of other compounds, besides avrainvillamide, that bind to, inhibit, interfere with, modulate, or activate this target. These identified compounds are also within the scope of the invention. In certain embodiments, the identified compounds are of the formula:

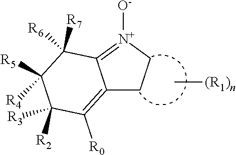

wherein

R₀, R₁, R₂, R₃, R₄, R₅, R₆, and R₇ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

wherein two or more substituents may form substituted or unsubstituted, cyclic, heterocyclic, aryl, or heteroaryl structures;

wherein R₂ and R₃, R₄ and R₅, or R₆ and R₇ may form together ═O, ═NR_{G}, or ═C(R_{G})₂, wherein each occurrence of R_{G} is defined as above;

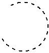

represents a substituted or unsubstituted, cyclic, heterocyclic, aryl, or heteroaryl ring system; and

n is an integer between 0 and 4. Other genera, subclasses, and species are described herein or in published PCT patent application, WO 2006/102097, which is incorporated herein by reference.

Nucleophosmin is also a validated target for identifying anti-proliferative and/or cytotoxic compounds useful in the treatment of such proliferative diseases as cancer, benign tumors, inflammatory diseases, diabetic retinopathy, infectious diseases, etc. The identified compounds are particularly useful in the treatment of cancer.

Nucleophosmin is highly conserved in vertebrates and widely distributed among different species with molecular weights ranging from 35 to 40 kDa. Nucleophosmin is a multifunctional protein that is overexpressed in many human tumors and has been implicated in cancer progression (Chan et al., Biochemistry 1989, 28, 1033-39; You et al., Naunyn-Schmiedeberg's Arch. Pharmacol. 1999, 360, 683-690; each of which is incorporated herein by reference). Primarily a nucleolar protein, nucleophosmin is widely expressed in metazoans and binds to many different proteins and nucleic acids as it shuttles between the nucleus and cytoplasm (Bertwistle et al., Mol. Cell. Biol. 2004, 24, 985-996; Kurki et al. Cancer Cell 2004, 5, 465-475; Grisendi, S.; Mecucci, C.; Falini, B.; Pandolfi, P. P. Nature Rev. Cancer 2006, 6, 493-505; Naoe, T.; Suzuki, T.; Kiyoi, H.; Urano, T. Cancer Sci. 2006, 97, 963-969; Lim, M. J.; Wang, X. W. Cancer Detect. Prev. 2006, 30, 481-490; Frehlick, L. J.; Eirín-López, J. M.; Ausió, J. BioEssays 2006, 29, 49-59; Gjerset, R. A. J. Mol. Hist. 2006, 37, 239-251; each of which is incorporated herein by reference). Nucleophosmin is frequently mutated in cancer cells. Genetic modifications of the C-terminal region of nucleophosmin are common in acute myeloid leukemia (AML) and are believed to be tumorigenic (Falini et al., N. Engl. J. Med. 2005, 352, 254-266; Falini et al., Blood 2007, 109, 874-885; each of which is incorporated herein by reference). More than half of anaplastic large-cell lymphomas (ALCLs) express a nucleophosmin-anaplastic lymphoma kinase fusion protein arising from a chromosomal translocation event, which is proposed to be transforming Different nucleophosmin fusion proteins have been identified in other cancers, and a 35-amino acid carboxyl-truncated form, NPM1.2, arising from alternative splicing, is associated with radiation insensitivity in HeLa cells and displays aberrant nuclear-cytosolic trafficking (Dalenc et al., Int. J. Cancer, 2002, 100, 662-668; Duyster et al., Oncogene 2001, 20, 5623-5637; Turner et al., Leukemia, 2005, 19, 1128-1134; Redner et al., Blood 1996, 87, 882-886; Yoneda-Kato et al. Oncogene 1996, 12, 265-275; each of which is incorporated herein by reference). Nucleophosmin is also deleted in certain tumors, although this is less common than its overexpression in tumor cells (Berger et al., Leukemia, 2006, 20, 319-320; incorporated herein by reference). The roles of nucleophosmin in cancer are complex, and a detailed understanding of these is presently evolving, as discussed in several recent reviews (Grisendi, S.; Mecucci, C.; Falini, B.; Pandolfi, P. P. Nature Rev. Cancer 2006, 6, 493-505; Naoe, T.; Suzuki, T.; Kiyoi, H.; Urano, T. Cancer Sci. 2006, 97, 963-969; Lim, M. J.; Wang, X. W. Cancer Detect. Prev. 2006, 30, 481-490; Frehlick, L. J.; Eirin-López, J. M.; Ausió, J. BioEssays 2006, 29, 49-59; Gjerset, R. A. J. Mol. Hist. 2006, 37, 239-251; each of which is incorporated herein by reference), but a significant factor is believed to be its ability to regulate the tumor suppressor protein p53 (Colombo et al., Nature Cell Biol. 2002, 4, 529-533; Maiguel et al., Mol. Cell. Biol. 2004, 24, 3703-3711; each of which is incorporated herein by reference). Among other findings, RNA silencing of nucleophosmin or disruption of its function by the addition of a small nucleophosmin-binding peptide (Szebeni et al. Biochemistry 1995, 34, 8037-8042; incorporated herein by reference) leads to increased expression of p53 (Chan et al., Biochem. Biophys. Res. Commun. 2005, 333, 396-403; incorporated herein by reference). Loss of p53 function (owing to mutation, deletion, or hDM2 overexpression) is one of the most common features of transformed cells, and novel approaches to restore cellular p53 function are widely sought as these have demonstrated potential for tumor regression in vivo (Hollstein et al., Science 1991, 253, 49-53; Vassilev et al., Science, 2004, 303, 844-848; Peng, Z. Hum. Gene Ther. 2005, 16, 1016-1027; each of which is incorporated herein by reference). The identification of nucleophosmin as a principle biological target of avrainvillamide provides a novel lead for the development of novel anti-cancer therapies.

Screening for Compounds that Target Nucleophosmin

The identification of nucleophosmin as a principle biological target of avrainvillamide makes possible an assay for use in identifying other compounds that inhibit, activate, bind to, or modify nucleophosmin. The compounds identified using the inventive screen are useful in the treatment of proliferative diseases such as cancer. In certain embodiments, the identified compounds modulates the expression and/or activity of the tumor suppressor protein p53 through nucleophosmin. The compounds may also modulate the expression and/or activity of nucleophosmin-binding proteins. In certain embodiments, the identified compounds modulate the expression and/or activity of hDM2/mDM2. In certain embodiments, the identified compounds modulate the expression and/or activity of p14ARF/p19ARF. In certain embodiments, the identified compounds affect nucleophosmin's ability to act as histone chaperone. In certain embodiments, the identified compounds affect nucleophosmin's ability to bind nucleic acids such as DNA or RNA. In certain embodiments, the identified compounds affect nucleophosmin's oligomerization state. In certain embodiments, the identified compounds affect nucleophosmin's phosphorylation state. The compounds identified using the inventive assay are considered part of the present invention. These compounds may or may not have structural similarity to avrainvillamide, stephacidin B, or the α,β-unsaturated nitrone-containing core of these molecules. In certain embodiments, the compounds are described herein and include the α,β-unsaturated nitrone-containing core of avrainvillamide. In certain embodiments, the compounds are of the formula:

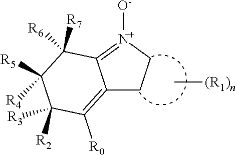

wherein

R₀, R₁, R₂, R₃, R₄, R₅, R₆, and R₇ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

wherein two or more substituents may form substituted or unsubstituted, cyclic, heterocyclic, aryl, or heteroaryl structures;

wherein R₂ and R₃, R₄ and R₅, or R₆ and R₇ may form together ═O, ═NR_{G}, or ═C(R_{G})₂, wherein each occurrence of R_{G} is defined as above;

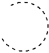

represents a substituted or unsubstituted, cyclic, heterocyclic, aryl, or heteroaryl ring system; and

n is an integer between 0 and 4.

The inventive assay includes (1) contacting at least one test compound with nucleophosmin, and (2) detecting an effect on nucleophosmin or an effect mediated by nucleophosmin. The assay may be adapted for high-throughput screening of test compounds. For example, multi-well plates, fluid-handling robots, plate readers, software, computers, etc. may be used to perform the assay on a plurality of test compounds in parallel.

In the inventive assay, a test compound is incubated with nucleophosmin. The assay may use any form of nucleophosmin. In certain embodiments, purified nucleophosmin is used. In other embodiments, partially purified or unpurified nucleophosmin is used. For example, cell lysates containing nucleophosmin may be used. The nucleophosmin protein used in the inventive assays may be derived from any species. In certain embodiments, mammalian nucleophosmin, preferably human nucleophosmin, is used. Nucleophosmin may be obtained from natural sources such as a cell line known to express nucleophosmin, or nucleophosmin may be obtained from recombinant sources such as bacteria, yeast, fungi, mammalian cells, or human cells made to overexpress nucleophosmin. The assay may use any isoform of nucleophosmin. In certain embodiments, the isoform of nucleophosmin used is NPM1.3, which contains a 29 amino acid deletion in the central, basic region of the peptide sequence (see Gene Bank Accession No. NM_{—}199185). In certain other embodiments, the isoform of nucleophosmin is NPM1.1. See Lim et al., Cancer Detection and Prevention 30:481-490, 2006; incorporated herein by reference.

In certain embodiments, rather than using purified or partially purified nucleophosmin, cells expressing nucleophosmin are used. Preferably, the cells are whole cells which are intact when incubated with the test compound. The cells may be any type of cell including cancer cell lines, mammalian cells, human cells, bacterial cells, yeast cells, etc. The cells may normally express nucleophosmin. In certain embodiments, the cells may overexpress nucleophosmin. In certain embodiments, the expression of nucleophosmin in the cells may be altered (e.g., increased or decreased) using any technique known in the art (see, for example, Sambrook et al., Molecular Cloning, second edition, Cold Spring Harbor Laboratory, Plainview, N.Y.; (1989), or Ausubel et al., Current Protocols in Molecular Biology, Current Protocols (1989), and DNA Cloning: A Practical Approach, Volumes I and II (ed. D. N. Glover) IREL Press, Oxford, (1985); each of which is incorporated herein by reference). For example, the expression of nucleophosmin may be increased by transfecting a cell line with a vector which constitutively or upon induction (e.g., addition of an inducing agent) expresses nucleophosmin. In other embodiments, the expression of nucleophosmin in the cell may be knocked down by siRNA. Wild type nucleophosmin protein may be used, a splice variant of nucleophosmin, an isoform of nucleophosmin, or a mutant form of nucleophosmin may be used in the inventive assay. In certain embodiments, certain amino acid of nucleophosmin may be mutated or deleted. In certain embodiments, a C275A mutant of nucleophosmin is used in the inventive assay. In certain embodiments, the N-terminal domain of nucleophosmin is used. In certain embodiments, the nucleophosmin used in the inventive assay comprises the N-terminal region. In certain embodiments, the C-terminal domain of nucleophosmin is used. In certain embodiments, the nucleophosmin used in the inventive assay comprises the C-terminal region. In certain embodiments, the nuclear signaling region of nucleophosmin is used. In certain embodiments, the nucleophosmin used in the inventive assay comprises the nuclear signaling region. In certain other embodiments, the nucleolar signaling region of nucleophosmin is used. In certain embodiments, the nucleophosmin used in the inventive assay comprises the nucleolar signaling region. In other embodiments, amino acids may be added to the nucleophosmin sequence (e.g., green fluorescent protein, a poly-histidine tag, an epitope, etc.).

The nucleophosmin and the test compound are contacted under any test conditions; however, conditions close to physiological conditions are preferred. For example, the test compound and nucleophosmin are contacted with each other at approximately 30-40° C., preferably at approximately 37° C. The pH may range from 6.5-7.5, preferably pH 7.4. Various salts, metal ions, co-factors, proteins, peptides, polynucleotides, etc. may be added to the incubation mixture.

After nucleophosmin has been incubated for a certain time with the test compound, it is then determined if the test compounds has had an effect on nucleophosmin or the cells expressing nucleophosmin. For example, the nucleophosmin protein may be assayed for binding to interacting proteins, binding to interacting nucleic acids, competition with known binders of nucleophosmin, alkylation, conformational changes, phosphorylation, etc. In certain embodiments, nucleophosmin is assayed for phosphorylation via immunoassay, radioactive assay using labeled phosphate, mass spectroscopy, etc. In other embodiments, covalent modification of nucleophosmin protein by the test compound is assayed for in the inventive assay. In certain embodiments, the compound is labeled with a radioactive isotope for detection. In other embodiments, the covalent modification of nucleophosmin may be detected via mass spectrometry. The effect of nucleophosmin on other biomolecules or pathways may also be determined. In certain embodiments, the effect on nucleophosmin-binding proteins is determined. In certain embodiments, the effect on p53 is determined. In certain embodiments, the effect on hDM2/mDM2 is determined. In certain embodiments, the effect on p14ARF/p19ARF is determined. In certain embodiments, the effect on nucleophosmin's ability to act as a histone chaperone is determined. In certain embodiments, the effect on nucleophosmin's ability to bind a nucleic acid is determined. In certain embodiments, the effect on nucleophosmin's oligomerization state is determined. The effect of the test compound may also be assessed by determining the effect on the cell expressing nucleophosmin. For example, the proliferation or inhibition of growth of the cells may be determined. In other embodiments, another phenotype of the cells may be determined for example, morphology of the ER, morphology of the cell, size of the cell, size of nucleus, DNA content, etc. In certain embodiments, localization or movement of nucleophosmin from the cytoplasm to the nucleus or nucleolus may be determined.

In certain embodiments, the inventive assay is a competition experiment. A compound of unknown binding to nucleophosmin is compared to a known binder of nucleophosmin. In certain embodiments, the known binder is an analogue of avrainvillamide. In certain embodiments, the known binder is a biotinylated probe of avrainvillamide or an analogue thereof. In certain embodiments, the biotinylated probe is of formula:

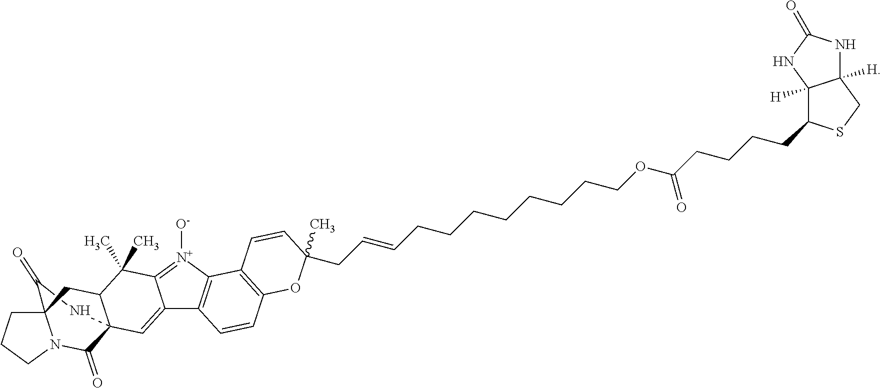

In certain embodiments, the biotinylated probe is of formula:

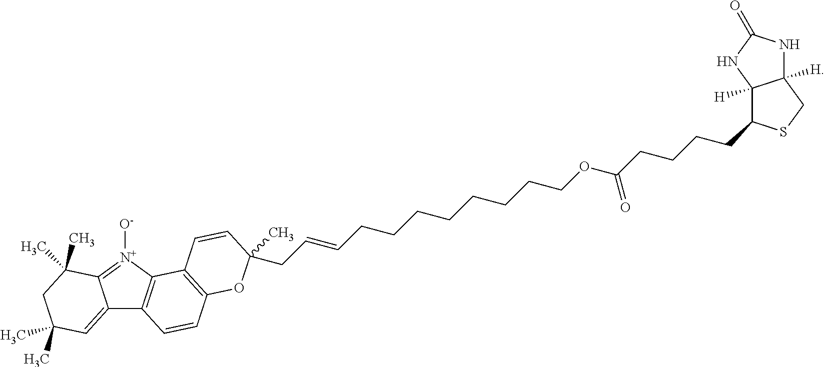

Test compounds are co-incubated with a known binder. Test compounds that bind strongly to the target will out-compete the labeled probe (e.g., biotinylated probe) from nucleophosmin's binding site. This effect can be detected by Western blot analysis. Test compounds that bind less efficiently will marginally affect binding between the probe and the target. In certain embodiments, the test compound is titrated over a range of concentrations to estimate the relative strength of binding for a series of small molecule-protein interactions.

In certain embodiments, an ELISA-based competition assay is used to identify binders of nucleophosmin. Nucleophosmin is immunoprecipitated in the presence of a fluorescent labeled known binder of nucleophosmin. In certain embodiments, the fluorescent labeled binder is avrainvillamide or an analogue thereof. In certain embodiments, the fluorescent labeled binder is of formula:

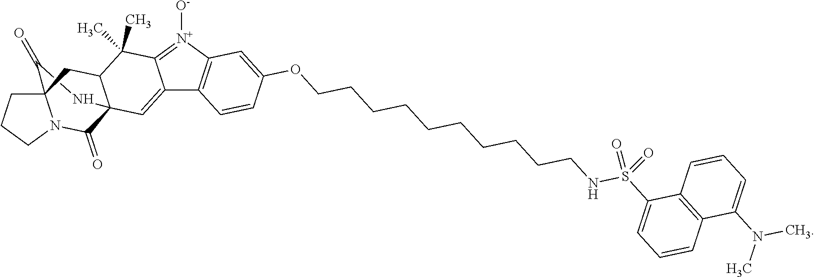

Addition of test compounds at various concentrations will allow one to estimate the relative binding efficiencies via fluorescent detection of the resulting complex.

In certain embodiments, the inventive assay is used to identify compounds that are specific for nucleophosmin. In certain embodiments, the identified test compounds do not bind or minimally bind CLIMP-63, glutathione reductase, peroxiredoxin 1, heat shock protein 60, or exportin 1. The inventive assay with minor modifications may also be used to identify compounds that target other possible biological targets of avrainvillamide such as, for example, CLIMP-63, glutathione reductase, peroxiredoxin 1, heat shock protein 60, or exportin 1. Instead of nucleophosmin, another possible target of avrainvillamide is used in the assay.

Any type of compound may be tested using the inventive assay including small molecules, peptides, proteins, polynucleotides, biomolecules, etc. In certain embodiments, the test compounds are small molecules. In certain embodiments, the small molecules have molecular weights less than 1500 g/mol. In certain embodiments, the small molecules have molecular weights less than 1000 g/mol. In other embodiments, the small molecules have molecular weights less than 500 g/mol. In other embodiments, the test compounds are peptides or proteins. In yet other embodiments, the test compounds are polynucleotides. In certain embodiments, the test compounds are biomolecules. In other embodiments, the test compounds are not biomolecules. The compounds to be tested in the inventive assay may be purchased, obtained from natural sources (i.e., natural products), obtained by semi-synthesis, or obtained by total synthesis. In certain embodiments, the test compounds are obtained from collections of small molecules such as the historical compound collections from the pharmaceutical industry. In certain embodiments, the test compounds are prepared using combinatorial chemistry. In other embodiments, the test compounds are prepared by traditional one-by-one chemical synthesis.

Once a compounds is identified as targeting nucleophosmin, it may be optionally further modified to obtain a compounds with greater activity and/or specificity for nucleophosmin. The compound may also be modified to obtain a compound with better pharmacological properties for use in administration to a subject (e.g., human).

### Methods of Treating Proliferative Diseases Based on Targeting Nucleophosmin

The identification of nucleophosmin as a principle biological target of avrainvillamide is the first demonstration of a small molecule that targets nucleophosmin in the treatment of proliferative diseases. Compounds that inferere with nucleophosmin, and specifically its effect on p53, are particularly useful in the treatment of proliferative diseases. Proliferative disorders include, but are not limited to, cancer, inflammatory diseases, graft-vs.-host disease, diabetic retinopathy, and benign tumors. In certain embodiments, compounds that target nucleophosmin may also be useful in the treatment of infectious diseases. In certain embodiments, the compounds described herein target nucleophosmin and are useful in the treatment of proliferative diseases or infectious diseases. Compounds that target nucleophosmin are administered in therapeutically effective doses to a subject suffering from a proliferative disease. In certain embodiments, the subject suffers from cancer. In certain embodiments, the subject suffers from an inflammatory disease (e.g., autoimmune diseases, rheumatoid arthritis, allergies, etc.). In certain embodiments, the subject suffers from an infectious disease (e.g., bacterial infection, fungal infection, protazoal infection, etc.).

A therapeutically effective amount of a compound that targets nucleophosmin is administered to a subject. In certain embodiments, 0.01-10 mg/kg of the compound is administered per day. In other embodiments, 0.01-5 mg/kg of the compound is administered per day. In yet other embodiments, 0.01-1 mg/kg of the compound is administered per day. The daily dose may be divided into several dosages taken within a twenty four hour period (e.g., twice a day, three times a day, four times a day, or more). The compound may be administered to the subject using any route known in the art as described above. In certain embodiments, the compound is administered orally. In other embodiments, the compound is administered parenterally. In yet other embodiments, the compound is administered intravenously.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### The Natural Product Avrainvillamide Binds to the Oncoprotein Nucleophosmin

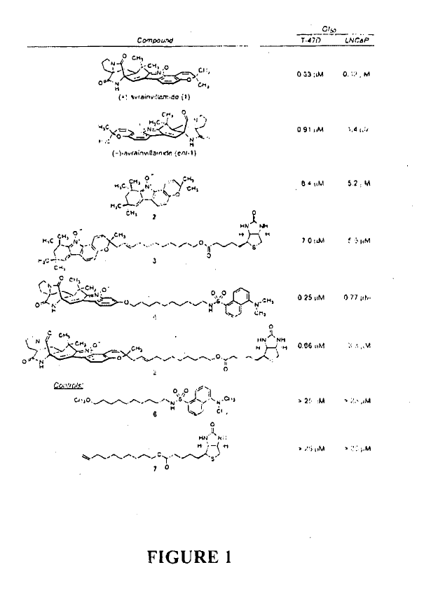

In an effort to determine the molecular basis of these effects, we employed the structurally simpler, less potent analogue 2, containing the 3-alkylidene-3H-indole 1-oxide (unsaturated nitrone) core of 1, and its biotin conjugate 3 (FIG. 1) to isolate and identify potential protein-binding partners from cancer-cell lysates (Wulff, J. E.; Herzon, S. B.; Siegrist, R.; Myers, A. G. J. Am. Chem. Soc. 2007, 129, 4898-4899; PCT Application, WO 2006/102097; each of which is incorporated herein by reference). Four proteins were thus identified (HSP60, XPO1, GR and PRX1), all containing active-site or known reactive cysteine residues. In each case, protein binding was inhibited in the presence of iodoacetamide, suggesting that the binding was cysteine-mediated, consistent with the earlier proposal that (+)-avrainvillamide and its analogues function as electrophiles by reversible, covalent nucleophilic (thiol) addition to the unsaturated nitrone functional group (Myers, A. G.; Herzon, S. B. J. Am. Soc. 2003, 125, 12080-12081; incorporated herein by reference).

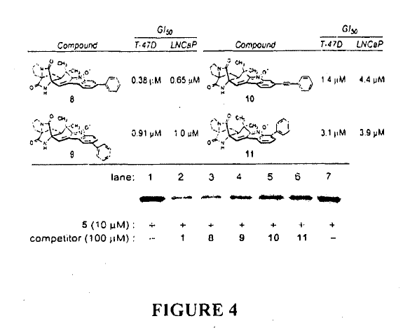

Prior studies revealed that (+)-avrainvillamide (1) has the capacity to bind one or more proteins in vitro, but did not establish to what degree the protein-small molecule interactions we had identified might contribute to the apoptotic events induced by (+)-avrainvillamide. Here, in studies using a series of molecules that more closely mimic the natural product 1 both structurally and in their growth-inhibitory activities (compounds 4 and 5, FIG. 1, and compounds 8-11, FIG. 4), we determine that (+)-avrainvillamide has the heretofore unrecognized capacity to bind to the nucleolar phosphoprotein nucleophosmin (NPM1.1, B23, numatrin, NO38), and provide evidence that this interaction contributes to the observed antiproliferative effects of (+)-avrainvillamide in cultured cancer cells. Site-directed mutagenesis experiments support the proposal that (+)-avrainvillamide binds specifically to cysteine-275 of nucleophosmin, a residue near the C-terminus and one of three free cysteines in the native protein.

While synthetic small molecules that bind to nucleophosmin and thereby inhibit its participation in protein-protein and/or protein-nucleic acid interactions might serve as potential leads for the development of novel anti-cancer therapies (Grisendi et al., Nature Rev. Cancer 2006, 6, 493-505; Naoe et al., Cancer Sci. 2006, 97, 963-969; Lim et al., Cancer Detect. Prev. 2006, 30, 481-490; Frehlick et al., BioEssays 2006, 29, 49-59; Gjerset, R. A. J. Mol. Hist. 2006, 37, 239-251; each of which is incorporated herein by reference), such compounds are largely unknown. In addition to the peptide ligand discussed in Szebeni, A.; Herrera, J. E.; Olson, M. O. J. Biochemistry 1995, 34, 8037-8042 and Chan et al., Biochem. Biophys. Res. Commun. 2005, 333, 396-403; actinomycin D (and related compounds) may bind to nucleophosmin. See: Busch, R. K.; Chan, P.-K.; Busch, H. Life Sci. 1984, 35, 1777-1785, incorporated herein by reference. Several cytotoxic compounds are known to cause translocation of nucleophosmin from the nucleolus to the nucleoplasm or to the cytoplasm, but a direct interaction has not generally been inferred. See: (a) Chan, P. K. Expt. Cell Res. 1992, 203, 174-181. (b) Lee, H.-Z.; Wu, C.-H.; Chang, S.-P. Int. J. Cancer 2005, 113, 971-976; (c) Yung, B. Y.-M.; Busch, H.; Chan, P.-K. Cancer Res. 1986, 46, 922-925; (d) Chan, P.-K.; Aldrich, M. B.; Yung, B. Y.-M. Cancer Res. 1987, 47, 3798-3801; each of which is incorporated herein by reference. The S-glutathionylation of nucleophosmin has also been reported, but the cysteine residue involved in this transformation was not determined. See Townsend, D. M.; Findlay, V. J.; Fazilev, F.; Ogle, M.; Fraser, J.; Saavedra, J. E.; Ji, X.; Keefer, L. K.; Tew, K. D. Molec. Pharm. 2006, 69, 501-508; incorporated herein by reference. Nucleophosmin has also been identified as a receptor for phosphatidylinositol lipids, which may contribute to its regulatory activity. See Ye, K. Cancer Biol. Ther. 2005, 4, 918-923; incorporated herein by reference. In contrast to the parent protein nucleophosmin, several inhibitors of the hybrid oncoprotein NPM-ALK have been identified, but these presumably act upon the kinase domain. For a recent example, see Galkin et al., Proc. Nat. Acad. Sci. USA 2007, 104, 270-275; incorporated herein by reference.

### Results and Discussion

By modifying one coupling partner in a late-stage, two component coupling reaction (step 15 of a 17-step synthetic sequence) (Herzon, S. B.; Myers, A. G. J. Am. Chem. Soc. 2005, 127, 5342-5344; incorporated herein by reference), we have prepared more than 30 analogues of avrainvillamide to date.

For this study, we made use of the dansyl- and biotin-conjugated probes 4 and 5, respectively, and the analogues 8-11 of FIG. 4. We first studied the antiproliferative effects of the conjugates 4 and 5 and found that both compounds inhibited the growth of T-47D (breast cancer) cells with potencies similar to the natural product (FIG. 1). Although the biotin conjugate (5) was somewhat less potent than the dansyl conjugate (4) in inhibiting the growth of LNCaP (prostate cancer) cells, it did provide a GI₅₀ value similar to values measured with the structurally simpler analogue 2 and its biotin conjugate 3, compounds we had previously studied and reevaluated herein as controls (Wulff et al., J. Am. Chem. Soc. 129:4898-4899, 2007; incorporated herein by reference). Compounds 6 and 7 (FIG. 1), which lack the unsaturated nitrone function but contain the dansyl and biotin groups, respectively, as well as the lipophilic tethering groups, were inactive in our assays, suggesting that neither the tethers nor the reporter groups of the active probes 3-5 contribute substantially to the observed antiproliferative activities of these compounds.

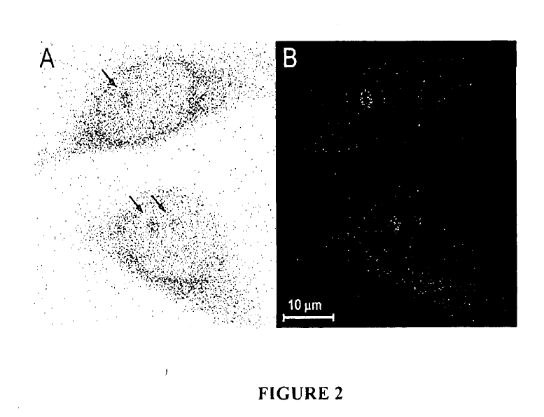

Fluorescence microscopy studies conducted with the dansyl-conjugate 4 revealed partial localization of the probe in the nucleoli of HeLa S3 (cervical cancer) and T-47D cells, in addition to a somewhat dispersed cytoplasmic distribution (FIG. 2).

To identify potential binding proteins, populations of healthy (adherent) T-47D cells were treated with the newly synthesized biotin conjugate 5 or the structurally simpler biotin-containing probe 3, previously studied. As a control, a separate population of cells was treated with the biotin derivative 7, which lacks the unsaturated nitrone function. The treated cells were incubated with probe or control for 90 min at 37° C., then were harvested, washed and lysed. The individual lysates were exposed to an agarose resin to remove nonspecific binding proteins. After centrifugation, the supernatants were then exposed to a streptavidin-agarose resin. This resin was collected by centrifugation and washed. Bound proteins were released by heat-denaturation, separated by SDS-PAGE, and analyzed by LC-MS/MS and Western-blot.

Nucleophosmin was initially identified by MS/MS sequencing of a pool of proteins of broad molecular weight range obtained using the structurally simpler probe 3. The analysis was complicated by the presence of a number of non-specific binding proteins, including structural proteins such as actin, tubulin, and myosin, as well as a number of biotinylated proteins, but the identification of nucleophosmin in probe-treated but not control protein samples was reproducible. With this information, MS/MS sequencing of a protein pool of somewhat narrower molecular weight range obtained using the more complex probe 5 also revealed a large peptide fragment with an amino acid sequence corresponding to nucleophosmin.

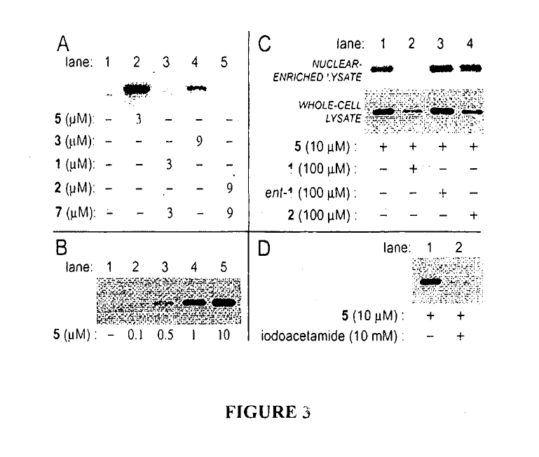

The presence of nucleophosmin in probe-derived (but not control) protein samples was readily confirmed by Western-blotting experiments (FIG. 3A, compare lane 2 with lane 3, and lane 4 with lane 5). Strikingly, probe 5 more effectively bound nucleophosmin than did the structurally simpler and less potent probe 3, even when a three-fold higher concentration of 3 was used relative to 5 (compare lane 2 of FIG. 3A with lane 4). This provided the first evidence that nucleophosmin might have a greater affinity for (+)-avrainvillamide (1) than for analogues with lesser potency in antiproliferative assays, such as 2 and 3. We found that as little as 100-500 nM concentrations of the biotinylated probe 5 were sufficient to afford detectable levels of nucleophosmin in affinity-isolation experiments from whole-cell lysates of T-47D cells (FIG. 3B). Competition experiments established that binding of nucleophosmin to the biotin-conjugated probe 5 in both nuclear-enriched and whole-cell lysates from T-47D cells was inhibited in the presence of a 10-fold higher concentration of free (+)-avrainvillamide (1) (FIG. 3C, compare lane 2 with lane 1), was not diminished in the presence of a 10-fold higher concentration of (−)-avrainvillamide (ent-1) (FIG. 3C, lane 3), and was somewhat diminished in the presence of a 10-fold higher concentration of the micromolar inhibitor 2 (FIG. 3C, lane 4). Binding of nucleophosmin to probe 5 was substantially reduced in the presence of a 1000-fold excess of iodoacetamide (FIG. 3D), consistent with the proposal that protein binding to 1 is cysteine-mediated (Wulff et al., J. Am. Chem. Soc. 129:4898-4899, 2007; incorporated herein by reference).

A more definitive series of competition experiments was conducted using a structurally similar series of analogues of (+)-1 spanning a 10-fold range of growth-inhibitory activities in T-47D and LNCaP cell lines (8-11, FIG. 4). The most active of these compounds (8) was nearly as potent as avrainvillamide in antiproliferative assays. The differing antiproliferative activities of compounds 8-11 were reasoned to be more likely attributable to differential target protein binding than to differential cell permeabilities and/or stabilities, although this was by no means certain. As shown by the data in FIG. 4, we observed a correlation between the antiproliferative activity of a compound and its ability to inhibit the affinity-isolation of nucleophosmin. Thus, binding of nucleophosmin to the probe was inhibited essentially equivalently by (+)-1 and the nearly equipotent analogue 8 (compare lanes 2 and 3, FIG. 4), was only partially inhibited by the 3-fold less potent inhibitor 9 (lane 4, FIG. 4), and was least effectively inhibited by the micromolar inhibitors 10 and 11 (lanes 5 and 6 of FIG. 4). (The correlation is not exact; for example, it appears that compound 11 is a slightly better inhibitor in the affinity-isolation of nucleophosmin than compound 10, although 10 is a more potent inhibitor of T-47D cell growth. This may well reveal the weakness of the underlying assumption that 10 and 11 will function equivalently in the many determinants of a measured GI₅₀ value (lipophilicity, transport, metabolism, etc.), which is not surprising.) This type of correlation was not observed with other proteins we had identified from our previous affinity-isolation experiments. For example, affinity-isolation of both exportin-1 and peroxiredoxin-1 from T-47D cells using the probe 5 is inhibited equally by (+)-1 and ent-1, although the latter is ˜3-fold less potent as an inhibitor of T-47D cell-growth. We have also identified an interaction in live cells between probe 3 and the endoplasmic reticulum protein CLIMP-63. See Myers et al., Synthesis of Avrainvillamide, Stephacidin B, and Analogues Thereof International PCT Application, PCT/US2006/009749, published as WO 2006/102097; which is incorporated herein by reference. Affinity-isolation experiments suggest that binding between probe 3 and CLIMP-63 is most pronounced after long incubation times (˜2 days). Preliminary experiments suggest that probes 3 and 5 do not display differential affinities for CLIMP-63. The observed difference in antiproliferative activity between (+)-1 and ent-(−)-1 appears to depend upon the assay conditions employed. In our previous report (Wulff et al., J. Am. Chem. Soc. 129:4898-4899, 2007), we made use of a 48-h incubation period, followed by detection with the MTS/PMS assay system. Under those conditions, (+)-1 was ˜9-fold more potent than the unnatural enantiomer. In the assay used here (72-h incubation period, followed by CellTiter-Blue detection), (+)-1 was only ˜3-fold more potent than ent-(−)-1. In contrast, inhibition of probe 5—nucleophosmin binding required the use of a ˜5-fold higher concentration of ent-1 versus 1 (500 μM and 100 μM, respectively).

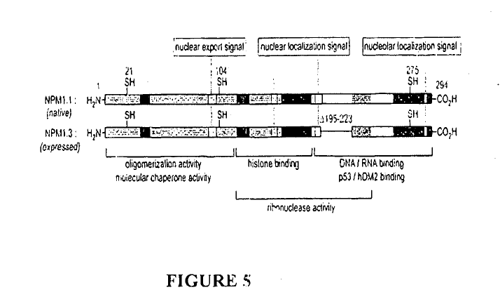

Wild-type nucleophosmin contains three free cysteine residues. Two of these, cys²¹ and cys¹⁰⁴, are located in the N-terminal domain, which serves as the locus for a dynamic pH- and ion-sensitive self-aggregation process leading to the formation of oligomeric complexes (Namboodiri et al., Structure 2004, 12, 2149-2160; Lee et al. 2007, in press. Structure available at www.pdb.org/pdb/explore.do?structureId=2P1B; Herrera et al., Biochemistry 1996, 35, 2668-2673; each of which is incorporated herein by reference). The C-terminal domain, which includes cys²⁷⁵, mediates interactions with p53, hDM2, and several known DNA and RNA sequences (Grisendi et al., Nature Rev. Cancer 2006, 6, 493-505; Naoe et al., Cancer Sci. 2006, 97, 963-969; Lim et al., Cancer Detect. Prev. 2006, 30, 481-490; Frehlick et al., BioEssays 2006, 29, 49-59; Gjerset, R. A. J. Mol. Hist. 2006, 37, 239-251). To identify whether a particular cysteine residue is involved in nucleophosmin binding, we prepared mutant constructs replacing in turn each cysteine residue with alanine, then expressed these mutant proteins in COS-7 cells. The mutant constructs were chosen to code for a naturally occurring (The cDNA for NPM1.3 was generated from isolates of a human large-cell lung carcinoma. Strausberg, R. L. et al. Proc. Natl. Acad. Sci. U.S.A. 2002, 99, 16899-16903; incorporated herein by reference.) isoform of nucleophosmin with a 29 amino acid-deletion in the central, basic region of the peptide sequence (NPM1.3, see FIG. 5; plasmids encoding both NPM1.1 and NPM1.3 are commercially available from Open Biosystems (Huntsville, Ala.)) in order to allow us to distinguish the mutant nucleophosmin proteins from the background native protein (NMP1.1). There is some confusion in the literature regarding the naming of this transcriptional variant of nucleophosmin. We use the convention of Lim et al. Cancer Detect. Prev. 30:481-90, 2006 in referring to this mutant, lacking alternate inframe exon 8 (Gene Bank accession # NM_{—}199185), as variant 3.

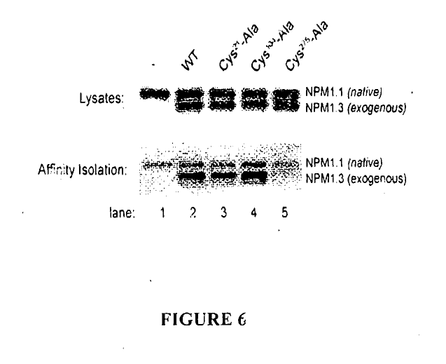

Following expression, the COS-7 cells were harvested and lysed. Affinity-isolation experiments were conducted as described above, using 1 μM biotinylated probe 5; nucleophosmin was detected by Western-blot analysis after separation by SDS-PAGE. As evident from the data of FIG. 6, NPM1.3 is readily distinguished from NPM1.1, and appears to be more effectively bound in the affinity-isolation procedure than the native form of the protein (NPM1.1). Whereas deletion of cys²¹ or cys¹⁰⁴ had little effect on affinity-isolation of NPM1.3 (compare lanes 3 or 4 of FIG. 6 with lane 2), deletion of cys²⁷⁵ greatly reduced affinity-isolation of NPM1.3 (compare lane 5 of FIG. 6 with lane 2), suggesting that cys²⁷⁵ mediates binding to the probe. The outcome of this experiment might well have been less definitive, given that nucleophosmin is known to self-associate to form oligomeric complexes;²⁹ this may explain the faint band for NPM1.3 that is present in lane 5 for the cys²⁷⁵→ala²⁷⁵ mutated protein.

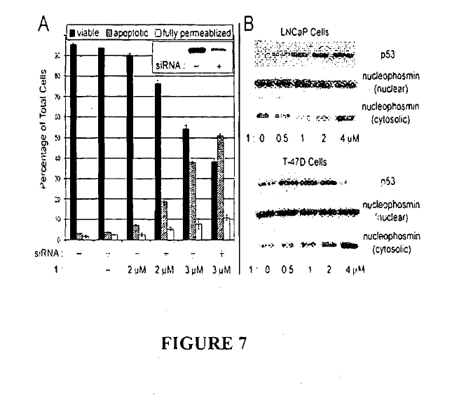

To further address the question of whether the binding of avrainvillamide to nucleophosmin may contribute to the observed antiproliferative effects of the natural product, we transiently depleted nucleophosmin in HeLa S3 cells by transfection with an siRNA targeting nucleophosmin, then compared the ability of (+)-avrainvillamide to induce apoptosis in the siRNA-modified cell line relative to a control population mock-transfected with a null siRNA (FIG. 7A). We found that the cells reduced in nucleophosmin exhibited enhanced sensitivity to (+)-avrainvillamide (1), providing a correlation between the antiproliferative effects of avrainvillamide and levels of the protein nucleophosmin.

Disruption of nucleophosmin function has been shown to lead to an increase in cellular p53 concentrations (Chan et al., Biochem. Biophys. Res. Commun. 333:396-403, 2005; incorporated herein by reference). We therefore investigated the effects of (+)-avrainvillamide-treatment on p53 levels in cultured cancer cells. Populations of healthy (adhered) T-47D or LNCaP cells were treated with varying concentrations of (+)-avrainvillamide (1) for 24 h. Following cell lysis and adjustment of concentrations to achieve uniform amounts of total protein, we analyzed for p53 by Western-blot. We observed a substantial increase in cellular p53 following the addition of as little as 500 nM (+)-avrainvillamide (1, see FIG. 7B). This increase occurs prior to apoptosis-related changes such as translocation of nucleophosmin to the cytosol (see FIG. 7B), cleavage of PARP, activation of caspase-3 or release of cytochrome-C from the mitochondrion (data not shown). Up-regulation of the tumor control-protein p53 is well known to promote apoptosis and is associated with tumor regression (Ventura et al., Nature 2007, 445, 661-665, incorporated herein by reference).

### Conclusion

(+)-Avrainvillamide (1) binds to a number of proteins in cancer cell lysates that contain reactive cysteine residues, as we have shown, and therefore may interact with more than one cellular protein in vivo. The discovery that avrainvillamide binds to nucleophosmin is significant, as non-peptidic small-molecules that bind this oncoprotein are virtually unknown. The apparent correlation we observe between the measured antiproliferative activities of a series of structurally similar analogues of avrainvillamide with their effectiveness in inhibiting the binding of nucleophosmin to the activity-based probe 5 is noteworthy. This, coupled with the finding that depletion of nucleophosmin by RNA silencing leads to increased sensitivity of HeLa S3 cells toward apoptotic cell death in the presence of (+)-avrainvillamide (1), suggests that the interaction of 1 and its analogues with cellular nucleophosmin may play a role in the observed antiproliferative effects of the compound class. The observation that affinity-isolation of nucleophosmin with the natural product-like probe 5 is inhibited in the presence of iodoacetamide is consistent with prior results that implicate avrainvillamide as an electrophile with a particular affinity for cysteine residues. Results of site-directed mutagenesis experiments, modifying in turn each of the three free cysteine residues of nucleophosmin, reveal that binding of the natural product is likely mediated by the specific residue cys²⁷⁵ near the C-terminus of the protein, which is associated with binding to nucleic acids and proteins such as p53 and hDM2.

### Experimental Section

A. Materials. (+)-Avrainvillamide (1), (−)-ent-avrainvillamide (ent-1), and compounds 2, 3 and 7 were synthesized as previously described (Wulff et al., J. Am. Chem. Soc. 129:4898-4899, 2007; Herzon, S. B.; Myers, A. G. J. Am. Chem. Soc. 2005, 127, 5342-5344; each of which is incorporated herein by reference). The syntheses of compounds 4-6 and 8-11 are described in the Supporting Information. LNCaP, T-47D, and HeLa-S3 cells were purchased from ATCC. COS-7 cells were a gift from the Alan Saghatelian group. Bradford reagent and Laemmli loading buffer (2× concentration) were purchased from Sigma Aldrich. Antiproliferative assays were conducted in pre-sterilized 96-well flat-bottomed plates from BD Falcon. Solutions of resazurin were purchased from Promega as the CellTiter-Blue Cell Viability Assay kit, and were used according to the manufacturer's instructions. Sodium dodecylsulfate polyacrylamide gel electrophoresis (SDS-PAGE) was performed using precast Novex Tris-glycine mini gels (10%, 12% or 4-20% gradient, Invitrogen). Benchmark prestained protein markers were purchased from Invitrogen. Electrophoresis and semi-dry electroblotting equipment was purchased from Owl Separation Systems. Nitrocellulose membranes were purchased from Amersham Biosciences. A mouse monoclonal antibody to nucleophosmin (B23) was purchased from Santa Cruz Biotechnology (sc-32256). A rabbit polyclonal antibody to peroxiredoxin 1 was purchased from GeneTex (GTX15571). Rabbit polyclonal antibodies to exportin 1 and p53 were purchased from Santa Cruz Biotechnology (XPO1: sc-5595; p53: sc-6243). An Alexafluor-647 goat anti-mouse secondary antibody, together with Image-iT FX Signal Enhancer blocking solution, was purchased from Invitrogen (A31625). Western-blot detection was performed using the SuperSignal West Pico Chemiluminscence kits (including goat anti-rabbit-HRP and goat anti-mouse-HRP conjugates) from Pierce. Western blots were visualized using CL-XPosure X-ray film from Pierce, or were imaged on an AlphaImager. Streptavidin-agarose and Sepharose 6B were purchased from Sigma Aldrich. Protein bands were visualized using the Novex Colloidal Blue staining kit from Invitrogen, and were analyzed at the Taplin Biological Mass Spectrometry Facility (Harvard University). Yo-Pro iodide was purchased from Invitrogen.

B. Instrumentation. Molecular Dynamics multiwell plate readers were used to obtain absorbance and fluorescence measurements (absorbance: SPECTRAmax PLUS 384, fluorescence: SPECTRAmax GEMINI XS). Data was collected using SOFTmax PRO v. 4.3 (Molecular Dynamics), and was manipulated in Excel (Microsoft). The XLfit4 plugin (IDBS software) running in Excel was used for curve fitting. Fluorescence microscopy experiments were performed using a Zeiss upright microscope, equipped with 355 nm, 488 nm, 543 nm and 633 nm lasers. Flow cytometry experiments were performed on an LSR II flow cytometer (BD Biosciences).

C. General Experimental Remarks. All cell-culture work was conducted in a class II biological safety cabinet. All buffers were filter-sterilized (0.2 nm) prior to use. Antiproliferative assays and other operations requiring the handling of nitrone species were carried out in the dark to prevent the occurrence of photochemical rearrangement reactions. Compounds 1-11 were stored at −80° C., either as frozen 5 mM stocks in DMSO, or as dry solids (100-μg portions).

D. Cell-Culture. Cells were cultured in RPMI 1640 (Roswell Park Memorial Institute culture medium, series 1640. For formulation, see Moore, G. E.; Gerner, R. E.; Franklin, H. A. JAMA 1967, 199, 519-524; incorporated herein by reference) (Mediatech) containing 10% fetal bovine serum (Hyclone), 10 mM HEPES, and 2 mM L-glutamine. Cells were grown in BD Falcon tissue culture flasks with vented caps.

E. Preparation of Solutions. RIPA buffer: 50 mM Tris.HCl, pH 7.35; 150 mM NaCl; 1 mM EDTA; 1% Triton X-100; 1% Sodium deoxycholate; 0.1% SDS; 1 mM PMSF; 5 μg/mL aprotinin; 5 μg/mL leupeptin; 200 nM Na₃VO₄; 50 mM NaF. Tris buffer: 50 mM Tris.HCl, pH 7.38; Wash buffer: 50 mM Tris.HCl, pH 7.6; 75 mM NaCl; 0.5 mM EDTA; 0.5% Triton X-100; 0.5% sodium deoxycholate; 0.05% SDS. Sucrose-hypotonic buffer: 25 mM Tris.HCl, pH 6.8; 250 mM sucrose; 0.05% digitonin; 1 mM DTT (dithiothreitol); 1 mM PMSF; 5 μg/mL aprotinin; 5 μg/mL leupeptin; 200 μM Na₃VO₄; 50 mM NaF. Apoptosis-detection buffer: 100 nM Yo-Pro iodide; 1.5 μM propidium iodide; 1 mM EDTA (ethylenediamine tetraacetic acid); 1% BSA (bovine serum albumin) in PBS (phosphate buffered saline) (Mediatech).

F. Preparation of Resins. A 400-μL aliquot of streptavidin-agarose suspension was transferred to a 1.7-mL centrifuge tube. Wash buffer (1.0 mL) was added, and the resulting slurry was mixed for 5 min at 4° C. The resin was isolated by centrifugation (12000×g, 2 min, 4° C.), and the supernatant was discarded. The resin was washed twice with 1.0 mL wash buffer (each wash: 5 min mixing at 4° C., followed by 2 min centrifugation at 12000×g, 4° C.), then was suspended in 800 μL wash buffer and mixed thoroughly prior to use. A 400-μL aliquot of Sepharose-6B suspension was treated identically, and used to remove nonspecific binding proteins where indicated.

G. Antiproliferative Assays. LNCaP and T-47D cells were grown to approximately 80% confluence, then were trypsinized, collected, and pelleted by centrifugation (10 min at 183×g). The cell pellet was suspended in fresh medium, and the concentration of cells was determined using a hemacytometer. The cell suspension was diluted to 1.0×10⁵ cells/mL. A multichannel pipette was used to load the wells of a 96-well plate with 100 μL per well of the diluted cell suspension. The plates were incubated for 24 h at 37° C. under an atmosphere of 5% CO₂. The following day, 100-μg portions of the nitrone samples were removed from the freezer, thawed, and dissolved in filter-sterilized DMSO to a concentration of 5 mM. A 6.5-μL aliquot of the nitrone solution was dissolved in 643.5 μL of medium to achieve a working concentration of 50 μM. Serial dilutions were employed to generate a range of different concentrations for analysis. Finally, 100-μL aliquots of this diluted nitrone solution were added to the wells containing adhered cells, resulting in final assay concentrations of up to 25 μM. The treated cells were incubated for 72 h at 37° C. (5% CO₂). To each well was added 20 μL of CellTiter-Blue reagent, and the samples were returned to the incubator. Fluorescence (560 nm excitation/590 nm emission) was recorded on a 96-well plate reader following a 4.0-h incubation period (37° C., 5% CO₂). Percent growth inhibition was calculated for each well, based upon the following formula: Percent growth inhibition=100×(S−B₀)/(Bₜ−B₀); where S is the sample reading, Bₜ is the average reading for a vehicle-treated population of cells at the completion of the assay, and B₀ is the average reading for an untreated population of cells at the beginning of the assay. Each analogue was run a minimum of eight times, over a period of at least two weeks. For each compound, 14 separate concentrations were used in the assay, ranging from 25 μM to 8 nM. The average inhibition at each concentration was plotted against concentration, and a curve fit was generated. To eliminate positional effects (e.g., cell samples in the center of the plate routinely grew more slowly than those near the edge), the data was automatically scaled to ensure that the curves showed no inhibition at negligible concentrations of added compound. Such a precaution was found to generate more consistent data from week to week, without affecting the final results. Final GI₅₀ values reflect the concentrations at which the resulting curves pass through 50 percent inhibition.

H. Fluorescence Microscopy Experiments. HeLa S3 cells adhered onto number 1.5 glass coverslips were exposed to medium containing 0 μM (vehicle control), 1 μM or 3 μM probe 4. All samples contained 0.06% DMSO. The samples were incubated (37° C., 5% CO₂) for 2 h, fixed in methanol at −20° C., and permeablized in 0.1% Triton X-100. The sample treated with 1 μM probe 4 was exposed to 150 μL of primary antibody solution (0.5 □L of mouse anti-B23 in 499.5 μL PBS), then to 150 μL of secondary antibody solution (0.5 □L of Alexafluor-647 goat anti-mouse in 499.5 μL PBS). All samples were washed with PBS and mounted with 20 μL Mowiol mounting mixture (containing 0.1% p-phenylene diamine) prior to analysis.

I. Identification of Nucleophosmin by LC-MS/MS. T-47D cells were grown to approximately 80% confluence, then were trypsinized, collected, and pelleted by centrifugation (10 min at 183×g). The supernatant was discarded, and the cell pellet was resuspended in fresh medium to achieve a concentration of approximately 1.0 to 1.5×10⁶ cells/mL. A sample was diluted 10-fold in fresh medium, and the concentration of cells was determined using a hemacytometer.

The cell suspension was diluted to 4.5×10⁵ cells/mL. Cell culture flasks (75 cm²) were charged with 12 mL of the suspension, and were then incubated for 2 days at 37° C. under an atmosphere of 5% CO₂.

The medium was removed from the growing cells, and replaced with 12 mL of medium containing either 8 μM of the biotinylated probe 3 or (as a control) 8 μM of compound 7. Incubation (at 37° C. and 5% CO₂) was continued for 1 d, after which the medium (including any detached cells) from each sample was transferred to a 50-mL centrifuge tube. The cells were rinsed with 10 mL PBS, which was added to centrifuge tubes. Adhered cells were detached from the culture flask by trypsinization (10 min, 37° C., 3 mL per flask, 0.05% trypsin, 0.53 mM EDTA). Fresh medium (6 mL) was added, and the resulting suspension was added to the centrifuge tubes, along with a 10 mL PBS rinse.

The samples were centrifuged (10 min at 183×g), and the supernatant was discarded. The cells were resuspended in 1 mL of PBS, transferred to a 1.5-mL centrifuge tube, and centrifuged again (5 min at 500×g). The supernatant was discarded, and the cells were washed with 1 mL of PBS.

The washed cells were cooled on ice, then lysed by addition of 500 μL per sample ice-cold RIPA buffer (see above for formulation). The samples were mixed end-over-end for 1 h at 4° C. with occasional vortexing, then 500 μL per sample Tris buffer was added. The samples were centrifuged (12000×g, 10 min, 4° C.), and insoluble material was removed with a pipette tip. The lysates were transferred to fresh 1.5-mL centrifuge tubes.

The protein concentration in each lysate was determined (Bradford method; Bradford, Anal. Biochem. 1976, 72, 248; incorporated herein by reference), and the samples were diluted with wash buffer to a final concentration of 3500 μg protein in 1100 μL. Each sample was treated with a 50-μL aliquot of washed, twice-diluted sepharose (see above for resin preparation) and the resulting slurry was mixed end-over-end for 1 h at 4° C. The samples were centrifuged (12000×g, 10 min, 4° C.), and 1 mL of supernatant from each sample was transferred to a clean 1.5-mL centrifuge tube. This was treated with two 30-μL aliquots of washed, well-suspended, two-fold diluted streptavidin-agarose resin (see above for resin preparation). The resulting slurry was mixed for 15 h at 4° C., then was centrifuged (12000×g, 10 min, 4° C.). The supernatant was discarded.

The collected resins were washed with wash buffer at 4° C., then with tris buffer at 4° C., then twice with tris buffer at 23° C. Each wash consisted of 10 min mixing, followed by 10 min centrifugation (either 12000×g at 4° C., or 10000×g at 23° C.). See above for solution preparation.

The washed resin was suspended in Laemmli loading buffer (Sigma, 2× concentration, 50 μL per sample), and the samples were heated to 95° C. for 6 min. A tris-glycine mini gel (10%, 12-well) was loaded with 20 μL per lane of the denatured protein mixture. The protein samples were electroeluted (20 min, 23° C., 150 V) until all of the loaded protein had migrated into the gel.

The resulting gel was stained with Colloidal Blue. The entire lanes (approximately 1 cm) corresponding to the protein from the two samples were submitted for protein sequencing by LC-MS/MS. Results are shown in Table S1.

TABLE 1
LC-MS/MS Analysis of Proteins Identified Following Affinity-Isolation with Probe 3

| | MW | percent coverage (by mass) | | |
|---|---|---|---|---|
| protein | (kDa) | 8 μM 3 | 8 μM 7 | assignment |
| cellular myosin heavy chain, type a | 226 | 40% | 41% | nonspecific binder: |
| | | | | myosin |
| actin-like protein Q562X8 | 12 | 28% | 28% | nonspecific binder: actin |
| actin-like protein actg1 | 29 | 18% | 18% | nonspecific binder: actin |
| actin-like protein Q562P9 | 11 | 17% | 17% | nonspecific binder: actin |
| 60s ribosomal protein l7 | 29 | 10% | — | possible selective |
| | | | | binding protein |
| cellular myosin heavy chain, type | 229 | 8% | 12% | nonspecific binder: |
| b | | | | myosin |
| tubulin alpha-2 chain | 50 | 8% | 4% | nonspecific binder: |
| | | | | tubulin |
| nucleophosmin | 33 | 7% | — | possible selective |
| | | | | binding protein |
| actin, alpha 1, skeletal muscle | 32 | 7% | 7% | nonspecific binder: actin |
| actin-like protein Q6ZSQ4 | 24 | 5% | 5% | nonspecific binder: actin |
| actin-like protein Q9BYX7 | 42 | 4% | 4% | nonspecific binder: actin |
| glyceraldehyde-3-phosphate | 36 | 4% | 10% | nonspecific binder: |
| dehydrogenase | | | | abundant protein |
| pyruvate kinase muscle isozyme | 58 | 4% | — | observed in other |
| | | | | experiments as a |
| | | | | nonspecific binding |
| | | | | protein |
| pyruvate carboxylase | 130 | 3% | 14% | biotinylated protein |
| tubulin alpha-6 chain | 50 | 3% | 3% | nonspecific binder: |
| | | | | tubulin |
| myosin heavy chain, smooth | 227 | 1% | 1% | nonspecific binder: |
| muscle isoform | | | | myosin |
| myosin heavy chain, nonmuscle iic | 228 | 1% | 1% | nonspecific binder: |
| | | | | myosin |
| methylcrotonoyl-coa carboxylase | 80 | — | 13% | biotinylated protein |
| subunit alpha | | | | |
| propionyl-coa carboxylase alpha | 77 | — | 4% | biotinylated protein |
| chain | | | | |
| propionyl-coa carboxylase beta | 58 | — | 3% | biotinylated protein |
| chain | | | | |
| methylcrotonoyl-coa carboxylase | 61 | — | 3% | biotinylated protein |
| beta chain | | | | |
| heat-shock protein beta-1 | 23 | — | 8% | known to associate with |
| | | | | tubulin |

Among several proteins common to both the sample and control lanes (in particular structural proteins such as myosin, actin, and tubulin, as well as biotinylated proteins), we observed only three proteins which were present in the sample originating from treatment with probe 3, but not in the control sample originating from treatment with compound 7. Of these, pyruvate kinase muscle isozyme was considered not to be a selective binding protein, since it had previously been detected in both sample and control lanes from other experiments.

In subsequent Western-blotting experiments, the 60 s ribosomal protein was likewise revealed to be a nonselective binding protein, while nucleophosmin was found to selectively bind to the biotinylated probes 3 and 5 (see below).

Attempts to directly identify nucleophosmin in a similar full-gel analysis by LC-MS/MS with the natural product-like probe 5 were unsuccessful (despite the fact that 5 binds more efficiently than 3 to nucleophosmin, as discussed below), as these analyses were invariably complicated by an overabundance of the nonspecific binding proteins discussed above. However, when a narrower region of the gel was submitted for analysis following affinity isolation with probe 5 and electroelution, nucleophosmin was detected by LC-MS/MS analysis. Nucleophosmin was not detected by LC-MS/MS analysis in control experiments using (+)-avrainvillamide (1) or 7 in lieu of probe 5 (equal concentrations).

J. Affinity-Isolation Experiments. Full details of affinity-isolation experiments in live cells and cellular lysates (including competitive binding experiments) are provided below.

For experiments in live cells, adhered T-47D cells were treated with probes (3 or 5) or controls (1, 2 and/or 7) in cell-culture medium for 90 min at 37° C. under an atmosphere of 5% CO₂. The medium (including any detached cells) from each sample was transferred to a 50-mL centrifuge tube. The cells were rinsed with 10 mL PBS, which was added to the centrifuge tubes. Adhered cells were detached from the culture flasks by trypsinization (10 min, 37° C., 5 mL per flask, 0.05% trypsin, 0.53 mM EDTA). Fresh medium (10 mL) was added, and the resulting suspension was added to the centrifuge tubes, along with a 5 mL PBS rinse. The samples were centrifuged (10 min at 183×g), and the supernatant was discarded. The cells were resuspended in 1 mL of PBS, transferred to a 1.7-mL centrifuge tube, and centrifuged again (5 min at 500×g). The supernatant was discarded, and the cells were washed twice with 1 mL of PBS. The washed cells were cooled on ice, then lysed by addition of 500 μL per sample ice-cold RIPA buffer. The samples were mixed end-over-end for 1 h at 4° C. with occasional vortexing, then 500 μL per sample Tris buffer was added. The samples were centrifuged (12000×g, 10 min, 4° C.), and insoluble material was removed with a pipette tip. The lysates were transferred to fresh 1.7-mL centrifuge tubes. Each individual sample lysate was treated with 50 μL of washed, well-suspended, two-fold diluted Sepharose resin. The resulting slurry was mixed for 6 h at 4° C., then was centrifuged (12000×g, 2 min, 4° C.). The supernatant was transferred to a clean 1.7-mL centrifuge tube.

For in vitro experiments, probe 5 was added (on ice, in the dark), in the presence or absence of competitors, to a 384-μL aliquot of cellular lysate at 1.5 mg/mL total protein (Bradford determination; Bradford, Anal. Biochem. 1976, 72, 248; incorporated herein by reference). The resulting samples (400 μL final volume, containing 4% DMSO) were mixed end-over-end in the dark for 4 h at 4° C.

Each sample was treated with two 30-μL aliquots of washed, well-suspended, two-fold diluted streptavidin-agarose resin. The resulting slurry was mixed for 15 h at 4° C., then was centrifuged (12000×g, 10 min, 4° C.). The supernatant was discarded. The collected resins were washed with wash buffer at 4° C., then with Tris buffer at 4° C., then twice with Tris buffer at 23° C. Each wash consisted of 10 min mixing, followed by 10-min centrifugation (either 12000×g at 4° C., or 10000×g at 23° C.). The washed resin was suspended in Laemmli loading buffer (70 μL per sample), and the samples were heated to 95° C. for 6 min. A Tris-glycine mini gel (4-20%, 12-well) was loaded with 15 μL per lane of the denatured protein mixture. The protein samples were electroeluted (1 h, 23° C., 150 V), then transferred under semi-dry conditions to a nitrocellulose membrane (100 mA, 23° C., 12 h). The membrane was blocked for 1 h (40 mL 3% low fat milk in TBS buffer with 0.1% Tween-20), then rinsed (two ten min washes with TBS buffer containing 0.1% Tween-20), and treated 1 h with primary antibody solution (20 mL of 1% low fat milk in TBS buffer with 0.1% Tween-20, containing 10 μg of mouse anti-B23 antibody). The membrane was rinsed again (two 10-min washes with 40 mL TBS buffer containing 0.1% Tween-20) and treated with secondary antibody solution (20 mL of 1% low-fat milk in TBS buffer with 0.1% Tween-20, containing 20 μg of goat anti-mouse-HRP conjugate). The membrane was rinsed once more (three ten min washes with 40 mL TBS buffer containing 0.1% Tween-20) and treated with 6 mL of a 1:1 mixture of stabilized peroxide solution:enhanced luminol solution for 3 min prior to visualization.

K. Site-Directed Mutagenesis and Transformation of COS-7 Cells. Site-Directed Mutagenesis Experiments.

### 1. Preparation of Mutant Sequences.

An E. coli DH10B clone carrying a pCMV-SPORT6 vector (including an ampicillin resistance gene) containing a cDNA that encodes for NPM1.3 was purchased from Open Biosystems (clone 3877633, catalogue number MHS1010-73718). A clone was streaked onto ampicillin-treated agar plates and incubated overnight at 37° C. The following day, individual colonies were selected and amplified overnight in 5 mL of ampicillin-containing broth. Plasmid DNA was isolated from individual colonies using the QIAGEN miniprep kit.

Cysteine→alanine mutations were carried out using the QuikChange Site-Directed Mutagenesis Kit (Invitrogen), following the manufacturer's directions. The following primers were used to effect the desired mutations:

Cys²¹ → Ala²¹: Forward primer:
(SEQ ID NO: XX)
5′-GCCCCAGAACTATCTTTTCGGTGCTGAACTAAAGGCCGAC-3′
Reverse primer:
(SEQ ID NO: XX)
5′-GTCGGCCTTTAGTTCAGCACCGAAAAGATAGTTCTGGGGC-3′
Cys¹⁰⁴ → Ala¹⁰⁴: Forward primer:
(SEQ ID NO: XX)
5′-TGGTCTTAAGGTTGAAGGCTGGTTCAGGGCCAGTGC-3′
Reverse primer:
(SEQ ID NO: XX)
5′-GCACTGGCCCTGAACCAGCCTTCAACCTTAAGACCA-3′
Cys²⁷⁵ → Ala²⁷⁵: Forward primer:
(SEQ ID NO: XX)
5′-AAGCCAAATTCATCAATTATGTGAAGAATGCCTTCCGGATGACTGA
C-3′
Reverse primer:
(SEQ ID NO: XX)
5′-GTCAGTCATCCGGAAGGCATTCTTCACATAATTGATGAATTTGGCT
T-3′

After codon exchange, the modified DNA was used to transform TOP10 chemically competent E. coli (Invitrogen) following the manufacturer's directions. The cells were plated on an ampicillin-treated agar plate and incubated overnight at 37° C. The following day, individual colonies were collected and amplified overnight in 5 mL of ampicillin-containing broth. Plasmid DNA was isolated (using the QIAGEN miniprep kit) and submitted for sequencing (Genewiz; forward primer=CACCATGGAAGATTCGATGGACATGG (SEQ ID NO: XX), reverse primer=TTAAAGAGACTTCCTCCACTGCC (SEQ ID NO: XX)).

Colonies expressing the desired plasmids were grown for 20 h at 37° C., in 50 mL of broth containing 100 μg/mL ampicillin. The following day, plasmid DNA was isolated (using the QIAGEN midiprep kit), quantified and sequenced (Genewiz).

### 2. Transformation of COS-7 Cells.

COS-7 cells were grown to approximately 80% confluence, then were trypsinized, collected, and pelleted by centrifugation (10 min at 183×g). The supernatant was discarded, the cell pellet was resuspended in fresh medium, and the concentration of the resulting suspension was determined using a hemacytometer.

Cell culture flasks (75 cm²) were charged with 12 mL of a 3×10⁵ cells/mL suspension, and incubated overnight at 37° C. under an atmosphere of 5% CO₂.

The following day, Lipofectamine 2000 (480 μL) was added to Opti-MEM reduced serum medium (3520 μL). Plasmid DNA (15 μg in QIAGEN extraction buffer) was added to Opti-MEM (to a final volume of 500 μL) for each sample (A: no DNA; B: NPM1.3; C: NPM1.3c²¹-a; D: NPM1.3c¹⁰⁴-a; E: NPM1.3c²⁷⁵-a). A 500-μL aliquot of the diluted Lipofectamine solution was added to each sample, and the resulting transfection complex solutions were incubated for 10 min at 23° C., then were diluted with 5 mL of Opti-MEM.

The medium was removed from the growing cells and replaced with the prepared transfection complex solutions. The samples were incubated at 37° C., under an atmosphere of 5% CO₂, for 5 h. The supernatant was removed from the adhered cells, and replaced with 12 mL of fresh serum-containing media. The samples were returned to incubation (37° C., 5% CO₂) for 60 h. The medium (including any detached cells) from each sample was transferred to a 50-mL centrifuge tube. The cells were rinsed with 10 mL PBS, which was added to centrifuge tubes. Adhered cells were detached from the culture flask by trypsinization (10 min, 37° C., 5 mL per flask, 0.05% trypsin, 0.53 mM EDTA). Fresh medium (10 mL) was added and the resulting suspension was added to the centrifuge tubes, along with a 5-mL PBS rinse.

The samples were centrifuged (10 min at 183×g), and the supernatant was discarded. The cells were resuspended in 1 mL of PBS, transferred to a 1.5-mL centrifuge tube, and centrifuged again (5 min at 500×g). The supernatant was discarded, and the cells were washed twice with 1 mL of PBS.

The washed cells were cooled on ice, then lysed by addition of 500 μL per sample ice-cold RIPA buffer (see above for formulation). The samples were mixed end-over-end for 1 h at 4° C. with occasional vortexing, then 500 μL per sample Tris buffer was added. The samples were centrifuged (12000×g, 10 min, 4° C.), and insoluble material was removed with a pipette tip. The lysates were transferred to fresh 1.5-mL centrifuge tubes. A 50-μL aliquot of washed, twice-diluted streptavidin-agarose resin (see above for wash conditions) was added to each sample, and the resulting slurry was rotated end-over-end for 15 h at 4° C. The samples were centrifuged (12000×g, 10 min, 4° C.), and the protein concentration in the supernatants was measured (Bradford method).

An aliquot from each supernatant was diluted with wash buffer to provide individual 397-μl samples, each containing 2 mg/mL total protein. These were mixed, then 5 μL was removed from each sample and added to Laemmli loading buffer (Sigma, 2× concentration, 45 μL per sample). The resulting solutions were heated to 95° C. for 6 mM, then were further diluted 5-fold with Laemmli loading buffer. A tris-glycine mini gel (12%, 12-well) was loaded with 15 μL per well of the diluted denatured protein mixture. The protein samples were electroeluted (150 V, 23° C., 90 min) and transferred to a nitrocellulose membrane (100 mA, 23° C., 12 h). Nucleophosmin (both native NPM1.1 and expressed NPM1.3) was detected by Western-blot using the procedure outlined above.

To the remaining 392-μL lysates, 8-μL aliquots of a 50 μM solution of probe 5 in DMSO were added (on ice, in the dark), to afford a final concentration of 1 μM probe 5, in each of the five 400-μL samples. The samples were mixed end-over-end at 4° C. for 4 h. Two 30-μL aliquot of washed, twice-diluted streptavidin-agarose resin (see above for wash conditions) was added to each sample, and the resulting slurry was rotated end-over-end for 15 h at 4° C.

The collected resins were washed with wash buffer at 4° C., then with tris buffer at 4° C., then twice with tris buffer at 23° C. Each wash consisted of 10 min mixing, followed by 10 min centrifugation (either 12000×g at 4° C., or 10000×g at 23° C.). See above for solution preparation.

The washed resin was suspended in Laemmli loading buffer (Sigma, 2× concentration, 50 μL per sample), and the samples were heated to 95° C. for 6 min. A tris-glycine mini gel (12%, 12-well) was loaded with 15 μL per well of the liberated protein mixture. The protein samples were electroeluted (150 V, 23° C., 90 min) and transferred to a nitrocellulose membrane (100 mA, 23° C., 12 h). Nucleophosmin (both native NPM1.1 and expressed NPM1.3) was detected by Western-blot using the procedure outlined above.

The results of the Western-blotting experiments (FIG. 5) suggest that cysteine-275 of nucleophosmin is required for binding to probe 5.

### Transfection/Apoptosis Experiments

HeLa S3 cells were grown to approximately 80% confluence, then were trypsinized, collected, and pelleted by centrifugation (10 min at 183×g). The supernatant was discarded, and the cell pellet was resuspended in fresh medium. The concentration of the cell suspension was determined using a hemacytometer, and a suspension of 1×10⁵ cells/mL was prepared.

siPORT NeoFX (100 μL) was added to Opti-MEM reduced serum medium (1900 μL). A siRNA targeting NPM1.1 (Applied Biosystems, Cat. No. AM16708; ID 143640; 11.4 μL from a 50 μM stock solution) was added to Opti-MEM (938.6 μL). At the same time, a control siRNA (Applied Biosystems, Cat. No. AM4611; 11.4 μL from a 50 μM stock) was similarly added to Opti-MEM (938.6 μL). A 950-μL aliquot of the diluted NeoFX solution was added to each sample, and the resulting transfection complex solutions were incubated for 10 min at 23° C.

Cell culture flasks (75 cm²) were charged with 1.8 mL of the prepared transfection complex solution, followed by 16.2 mL of the HeLa S3 cell suspension (at 1×10⁵ cells/mL). The samples were incubated for 2 d at 37° C., under an atmosphere of 5% CO₂. At the end of this period, the cells (which had reached ˜90% confluence) were stripped of media, rinsed with trypsin buffer, then detached from the culture flasks by trypsinization (5 min, 37° C., 5 mL per flask, 0.05% trypsin, 0.53 mM EDTA). Fresh medium (10 mL) was added and the resulting suspensions were transferred quantitatively to 50-mL centrifuge tubes. The culture flasks were rinsed with an additional 5 mL medium, which was likewise added to the centrifuge tubes.

The samples were centrifuged (10 min at 183×g). The supernatant was discarded, and the cells were resuspended in 30 mL per sample of fresh medium. The concentration of the cell suspensions was determined using a hemacytometer. Over the course of the 2 d transfection period, both the transfected and mock-transfected cells grew ˜4-fold. No statistically significant difference in growth rate was observed for the two populations of cells in this experiment, or in several related experiments, using various means of measurement (counting by hemacytometer, assaying cell viability with CellTiter-Blue, and quantifying total protein in lysed cells).

12-well plates were charged with 3 mL per well of suspensions of the transfected or mock-transfected cells, at 2.5×10⁴ cells per mL. The samples were incubated overnight at 37° C., under an atmosphere of 5% CO₂. The following day, solutions of cell culture medium containing (+)-avrainvillamide or vehicle control were prepared. 500-μL aliquots of these solutions were added to the 3-mL samples. The treated samples were returned to the incubator (37° C., 5% CO₂) for 24 h.

The medium (containing any detached cells) from each sample was transferred to a 15-mL centrifuge tube. The cells were rinsed with 1 mL PBS, which was added to the centrifuge tubes. Adhered cells were detached from the 12-well plates by trypsinization (5 min, 37° C., 300 μL per sample, 0.05% trypsin, 0.53 mM EDTA). The trypsin was quenched by the addition of 1 mL fresh medium, and the resulting suspension was added to the centrifuge tubes, along with a 1 mL rinse (PBS, with 1 mM EDTA and 1% BSA).

The samples were centrifuged (10 min at 183×g), and the supernatant was discarded. The cells from each sample were resuspended in 1 mL PBS (containing 1 mM EDTA and 1% BSA), transferred to a 1.5-mL centrifuge tube, and centrifuged again (5 min at 500×g). The supernatant was discarded, and the samples were cooled on ice. Apoptosis detection buffer (500 μL; see above for preparation) was added to each sample. The resulting suspensions were mixed and incubated on ice for 1 h, prior to analysis.

Each sample was analyzed on an LSRII flow cytometer, with 20,000 events recorded per sample. Apoptotic cells were defined as those permeable to Yo-Pro iodide, but not to propidium iodide (PI). Viable cells were defined as those permeable to neither die. Compensation controls were set manually, to achieve the greatest distinction between viable and apoptotic cell populations (PI vs. Yo-Pro: 30%; Yo-Pro vs. PI: 2%). The results (FIG. 6A) indicate that the transfected cells were more susceptible to avrainvillamide-induced apoptosis.

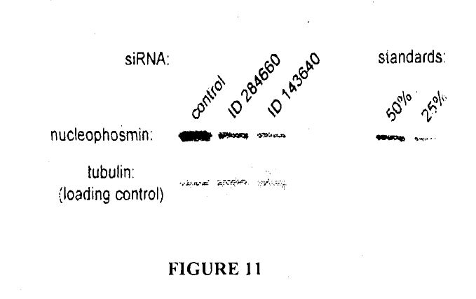

The experiment was carried out three times, with qualitatively similar results each time. Attempts to replicate these results with a second siRNA (Applied Biosystems, Cat. No. AM16708; ID 284660) were unsuccessful; Western-blotting experiments suggest that this siRNA afforded less complete suppression of nucleophosmin (FIG. 11).

L. Transfection/Apoptosis Experiments. HeLa S3 cells were grown to approximately 80% confluence, then were trypsinized, collected, and pelleted by centrifugation (10 min at 183×g). The supernatant was discarded, and the cell pellet was resuspended in fresh medium. The concentration of the cell suspension was determined using a hemacytometer, and a suspension of 1×10⁵ cells/mL was prepared. siPORT NeoFX (100 μL) was added to Opti-MEM reduced serum medium (1900 μL). A siRNA targeting NPM1.1 (Applied Biosystems, Cat. No. AM16708; ID 143640; 11.4 μL from a 50 μM stock solution) was added to Opti-MEM (938.6 μL). At the same time, a control siRNA (Applied Biosystems, Cat. No. AM4611; 11.4 μL from a 50 μM stock) was similarly added to Opti-MEM (938.6 μL). A 950-μL aliquot of the diluted NeoFX solution was added to each sample, and the resulting transfection complex solutions were incubated for 10 min at 23° C. Cell culture flasks (75 cm²) were charged with 1.8 mL of the prepared transfection complex solution, followed by 16.2 mL of the HeLa S3 cell suspension (at 1×10⁵ cells/mL). The samples were incubated for 2 d at 37° C., under an atmosphere of 5% CO₂. At the end of this period, the cells (which had reached ˜90% confluence) were stripped of media, rinsed with trypsin buffer, then detached from the culture flasks by trypsinization (5 min, 37° C., 5 mL per flask, 0.05% trypsin, 0.53 mM EDTA). Fresh medium (10 mL) was added, and the resulting suspensions were transferred quantitatively to 50-mL centrifuge tubes. The culture flasks were rinsed with an additional 5 mL medium, which was likewise added to the centrifuge tubes. The samples were centrifuged (10 min at 183×g). The supernatant was discarded, and the cells were resuspended in 30 mL per sample of fresh medium. The concentration of the cell suspensions was determined using a hemacytometer. Over the course of the 2 d transfection period, both the transfected and mock-transfected cells grew ˜4-fold. No statistically significant difference in growth rate was observed for the two populations of cells in this experiment, or in several related experiments, using various means of measurement (counting by hemacytometer, assaying cell viability with CellTiter-Blue, and quantifying total protein in lysed cells). 12-well plates were charged with 3 mL per well of suspensions of the transfected or mock-transfected cells, at 2.5×10⁴ cells/mL. The samples were incubated overnight at 37° C., under an atmosphere of 5% CO₂. The following day, solutions of cell culture medium containing (+)-avrainvillamide (1) or vehicle control were prepared. 500-μL aliquots of these solutions were added to the 3-mL samples. The treated samples were returned to the incubator (37° C., 5% CO₂) for 24 h. The medium (containing any detached cells) from each sample was transferred to a 15-mL centrifuge tube. The cells were rinsed with 1 mL PBS, which was added to the centrifuge tubes. Adhered cells were detached from the 12-well plates by trypsinization (5 min, 37° C., 300 μL per sample, 0.05% trypsin, 0.53 mM EDTA). Fresh medium (1 mL) was added, and the resulting suspension was added to the centrifuge tubes, along with a 1 mL rinse (PBS, with 1 mM EDTA and 1% BSA). The samples were centrifuged (10 min at 183×g), and the supernatant was discarded. The cells from each sample were resuspended in 1 mL PBS (containing 1 mM EDTA and 1% BSA), transferred to a 1.7-mL centrifuge tube, and centrifuged again (5 min at 500×g). The supernatant was discarded, and the samples were cooled on ice. Apoptosis detection buffer (500 μL) was added to each sample. The resulting suspensions were mixed and incubated on ice for 1 h, prior to analysis. Each sample was analyzed on an LSRII flow cytometer, with 20,000 events recorded per sample. Apoptotic cells were defined as those permeable to Yo-Pro iodide, but not to propidium iodide (PI). Viable cells were defined as those permeable to neither die. Compensation controls were set manually, to achieve the greatest distinction between viable and apoptotic cell populations (PI vs. Yo-Pro: 30%; Yo-Pro vs. PI: 2%). The experiment was carried out three times, with qualitatively similar results obtained each time. Attempts to replicate these results with a second siRNA (Applied Biosystems, Cat. No. AM16708; ID 284660) were unsuccessful; Western-blotting experiments suggest that this siRNA afforded less complete suppression of nucleophosmin (FIG. 11).

M. Effect of (+)-Avrainvillamide Incubation on p53/Nucleophosmin.
1. Treatment of Cells with (+)-Avrainvillamide

LNCaP and T-47D cells were grown to approximately 80% confluence, then were trypsinized, collected, and pelleted by centrifugation (10 min at 183×g). The supernatant was discarded, and the cell pellets were resuspended in fresh medium. The cell concentration in the resulting suspension was determined using a hemacytometer.

Four 6-well plates (two for each cell line) were charged with 6 mL per well of cell suspension at 2×10⁵ cells/mL. The cells were incubated overnight at 37° C., under an atmosphere of 5% CO₂. The following day, stock solutions of (+)-avrainvillamide (1) in fresh cell culture medium were prepared as indicated below:

| sample: | 1 | 2 | | 3 | | 4 | | 5 |
|---|---|---|---|---|---|---|---|---|
| DMSO: | 22.32 μL | 19.53 | μL | 16.74 | μL | 11.16 | μL | x |
| volume | x | 2.79 | μL | 5.58 | μL | 11.16 | μL | 22.32 μL |
| (+)-avrainvillamide (1): | | | | | | | | |
| (5 mM in DMSO) | | | | | | | | |
| Medium: | 877.68 μL | 877.68 | μL | 877.68 | μL | 877.68 | μL | 877.68 μL |
| [1] x 200/6200: | x | 0.5 | μM | 1 | μM | 2 | μM | 4 μM |

| | | | | | |
|---|---|---|---|---|---|
| [DMSO] x 200/6200: | 0.08% | 0.08% | 0.08% | 0.08% | 0.08% |

To each 6-mL sample, a 200-μL aliquot of the appropriate stock solution was added, resulting in a final concentration of 0-4 μM (+)-avrainvillamide (1). The samples were returned to the incubator (37° C., 5% CO₂) for 24 h.

The following day, the medium (containing any detached cells) from each sample was transferred to a 15-mL centrifuge tube. The cells were rinsed with 1 mL PBS, which was added to the centrifuge tubes. Adhered cells were detached from the 12-well plates by trypsinization (5 min, 37° C., 500 μL, per sample, 0.05% trypsin, 0.53 mM EDTA). Fresh medium (1 mL) was added and the resulting suspension was added to the centrifuge tubes, along with a 2-mL rinse with PBS.

The samples were centrifuged (10 min at 183×g), and the supernatant was discarded. The cells from each duplicate sample were combined (such that each sample contained the cells from two wells of a 6-well plate), then were resuspended in 1 mL PBS and transferred to a 1.5-mL centrifuge tube and centrifuged again (5 min at 500×g). The supernatant was discarded, and the cells were washed with 1 mL PBS. The cells were resuspended in 1 mL PBS and mixed thoroughly. A 500-μL aliquot from each sample was transferred to a fresh 1.5-mL centrifuge tube. All the samples were centrifuged (5 min at 500×g) and the supernatant was discarded. The resulting 20 samples (10 samples of T-47D cells, treated with 0-4 μM (+)-avrainvillamide, and 10 samples of LNCaP cells, treated with 0-4 μM (+)-avrainvillamide (1), where each sample contained the number of cells from 1 well of a 6-well plate) were separated into two groups. One group of samples was lysed in RIPA buffer (see below) to prepare a series of whole-cell lysates. The other group of samples was first treated with sucrose-hypotonic buffer to prepare a series of cytosolic lysates. The remaining pellets were washed and treated with RIPA buffer to prepare a series of nuclear-enriched lysates (see below).

### 2. Preparation and Analysis of Whole-Cell Lysates

From the samples prepared in section 1, five samples of T-47D cells and five samples of LNCaP cells (each treated with 0-4 μM (+)-avrainvillamide) were cooled on ice, treated for 1 h with ice-cold RIPA buffer (100 μL, see above for formulation), then centrifuged (12000×g, 10 min, 4° C.). The protein concentration in each lysate was quantified (Bradford method; samples and standards were measured in triplicate), and the lysates were mixed 1:1 with Laemmli loading buffer (Sigma, 2× concentration). The resulting samples were heated to 95° C. for 6 min, then were cooled and loaded onto tris-glycine mini gels (4-20%, 12-well) at 16 μg per well. The protein samples were electroeluted (1 h, 23° C., 150 V), then transferred under semi-dry conditions to nitrocellulose membranes (100 mA, 23° C., 12 h). The membranes were subjected to Western-blotting conditions for the detection of nucleophosmin, p53 and 14-3-3b (as a loading control), using an identical procedure to that described above.

### 3. Preparation and Analysis of Cytosolic and Nuclear-Enriched Lysates

From the samples prepared in section 1, five samples of T-47D cells and five samples of LNCaP cells (each treated with 0-4 μM (+)-avrainvillamide) were cooled on ice, and treated for 1 min with ice-cold sucrose-hypotonic buffer (50 μL, see above for formulation). The samples were vortexed and centrifuged (6800×g, 3 min, 4° C.). The supernatants (cytosolic lysates) were carefully transferred to fresh 1.5-mL centrifuge tubes. The remaining pellets were washed twice (on ice) twice with 500 μL PBS. The washed pellets were lysed by addition of ice-cold RIPA buffer (50 μL, see above for formulation). The resulting nuclear-enriched lysates were incubated 1 h at 4° C., then centrifuged (12000×g, 10 min, 4° C.).

The protein concentration in each lysate (both cytosolic and nuclear-enriched) was quantified (Bradford method; samples and standards were measured in triplicate), and the lysates were mixed 1:1 with Laemmli loading buffer (Sigma, 2× concentration). The resulting samples were heated to 95° C. for 6 min, then were cooled and loaded onto tris-glycine mini gels (4-20%, 12-well) at 16 μg per well. The protein samples were electroeluted (1 h, 23° C., 150 V), then transferred under semi-dry conditions to nitrocellulose membranes (100 mA, 23° C., 12 h). The membranes were subjected to Western-blotting conditions for the detection of nucleophosmin, p53 and 14-3-3β (as a loading control), using an identical procedure to that described above.

The results from these experiments (FIG. 6B, text, and S5, below) revealed an increasing concentration of p53 with increasing concentrations of (+)-avrainvillamide (1). The increase was observed in both T-47D cells (which have a relatively high concentration of p53 in unmodified cells) and LNCaP cells (which have a lower starting concentration of p53). Following incubation at the highest concentration of (+)-avrainvillamide (1), 4 μM, the T-47D cells experienced a reduction in cellular p53, presumably indicating proteasomal destruction of this protein as part of an apoptosis-related mechanism. The total concentration of nucleophosmin did not change, but translocation of nucleophosmin to the cytosol was observed following incubation with 4 μM (+)-avrainvillamide (1).

### A. Chemistry

General Experimental Procedures. All reactions were performed in single-neck, flame-dried, round-bottom flasks fitted with rubber septa under a positive pressure of argon, unless otherwise noted. Air- and moisture-sensitive liquids were transferred via syringe or stainless steel cannula. Organic solutions were concentrated at ambient temperature (23° C.) by rotary evaporation at 40 Torr (house vacuum). Analytical thin-layer chromatography (TLC) was performed using glass plates pre-coated with silica gel (0.25 mm, 60 Å pore-size, 230-400 mesh, Merck KGA) impregnated with a fluorescent indicator (254 nm). TLC plates were visualized by exposure to ultraviolet light, then were stained with iodine or by submersion in aqueous ceric ammonium molybdate (CAM), followed by brief heating on a hot plate. Flash-column chromatography was performed as described by Still et al. (Still et al. J. Org. Chem. 1978, 43, 2923; incorporated herein by reference), employing silica gel (60 Å, 32-63 μM, standard grade, Sorbent Technologies).

Materials. Commercial solvents and reagents were used as received with the following exceptions. Dichloromethane, benzene, tetrahydrofuran, and acetonitrile were purified by the method of Pangborn et al. (Organometallics 1996, 15, 1518; incorporated herein by reference). Biotinylated alkene 7 (Wulff et al., J. Am. Chem. Soc. 2007, 129, 4898; incorporated herein by reference), iodoarene 12 (Wulff et al., J. Am. Chem. Soc. 2007, 129, 4898; incorporated herein by reference), vinyl iodide 14 (Herzon et al. J. Am. Chem. Soc. 2005, 127, 5342; incorporated herein by reference), nitroarene 30 (Liu, L.; Zhang, Y.; Xin, B. J. Org. Chem. 2006, 71, 3994; incorporated herein by reference), iodoarene 34 (Maya, F.; Chanteau S. H.; Cheng L.; Stewart M. P.; Tour J. M. Chem. Mater. 2005, 17, 1331; incorporated herein by reference), and nitroaniline 36 (Seko, S.; Miyake, K.; Kawamura, N. J. Chem. Soc., Perkin Trans. 1 1999, 1437; incorporated herein by reference) were prepared as described previously.

Instrumentation. Proton nuclear magnetic resonance spectra (¹H NMR) were recorded at 400 or 500 MHz at 23° C. Proton chemical shifts are expressed in parts per million (ppm, δ scale) downfield from tetramethylsilane, and are referenced to residual protium in the NMR solvent (CHCl₃, δ 7.26; C₆HD₅, δ 7.15). Data are represented as follows: chemical shift, multiplicity (s=singlet, d=doublet, t=triplet, q=quartet, sext=sextet, m=multiplet and/or multiple resonances, br=broad, app=apparent), integration, and coupling constant in Hertz. Carbon nuclear magnetic resonance spectra (¹³C NMR) were recorded at 100 or 125 MHz at 23° C. unless otherwise noted. Carbon chemical shifts are reported in parts per million downfield from tetramethylsilane and are referenced to the carbon resonances of the solvent (CDCl₃, δ 77.0; C₆D₆, δ 128.0) Infrared (IR) spectra were obtained using a Perkin-Elmer FT-IR spectrometer referenced to a polystyrene standard. Data are represented as follows: frequency of absorption (cm⁻¹), intensity of absorption (s=strong, m=medium, w=weak, br=broad). Low- and high-resolution mass spectra were obtained at the Harvard University Mass Spectrometry Facility.

### Synthetic Procedures

For clarity, intermediates that have not been assigned numbers in the text are numbered sequentially in the supporting information, beginning with 12.

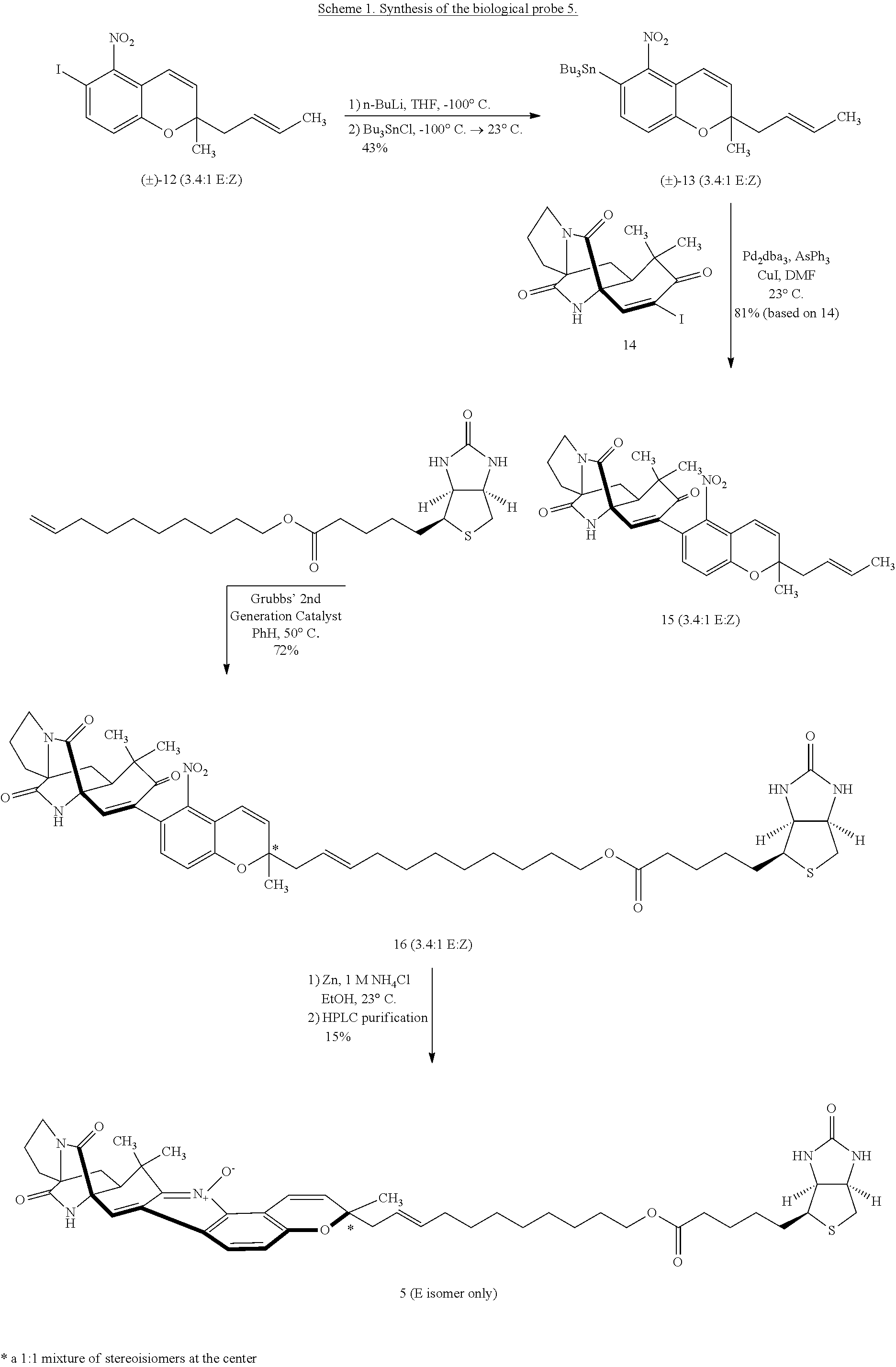

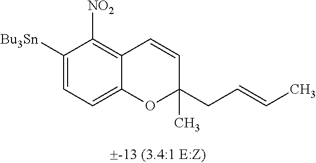

Stannane 13. n-Butyllithium in hexanes (2.4 M, 0.44 mL, 1.05 mmol, 1.05 equiv) and tributyltin chloride (0.28 mL, 1.05 mmol, 1.05 equiv) were added in sequence to a solution of the iodoarene 12 (371 mg, 1.0 mmol, 1.00 equiv) in tetrahydrofuran (10 mL) cooled to −100° C. The cooling bath was removed and the dark red solution was allowed to warm to 23° C. over 45 min. The solution was diluted with hexanes-ethyl ether (2:1) and the diluted solution was washed successively with water and saturated aqueous sodium chloride solution. The washed solution was dried over anhydrous sodium sulfate, the solids were removed by filtration, and the filtrate was concentrated in vacuo. The residue was purified by flash-column chromatography on silica gel (deactivated with 20% triethylamine-ethyl acetate, eluting with hexanes-ethyl acetate, 100:1), furnishing the stannane 13 (3.4:1 mixture of E- and Z-geometrical isomers, respectively, 228 mg, 43%) as an orange oil.

R_{f}=0.68 (hexanes-acetone 100:4). ¹H NMR (500 MHz, CDCl₃, signals for the major isomer), δ 7.26 (d, 1H, J=7.8 Hz), 6.98 (d, 1H, J=7.8 Hz), 6.68 (d, 1H, J=10.3 Hz), 5.77 (d, 1H, J=10.3 Hz), 5.55-5.43 (m, 2H), 2.49-2.36 (m, 2H), 1.66 (d, 3H, J=5.4 Hz), 1.57-1.38 (m, 6H), 1.41 (s, 3H), 1.32 (sext, 6H, J=7.3 Hz), 1.14-1.01 (m, 6H), 0.88 (t, 9H, J=7.3 Hz). ¹³C NMR (100 MHz, CDCl₃, signals for the major isomer), δ 154.8, 153.0, 136.9, 132.8, 129.8, 127.7, 124.9, 120.7, 118.5, 116.0, 78.4, 44.1, 29.2, 27.5, 25.9, 18.3, 13.9, 10.9. IR (NaCl, thin film), cm⁻¹ 2957 (m), 2921 (m), 2872 (m), 2854 (m), 1522 (s), 1279 (s).

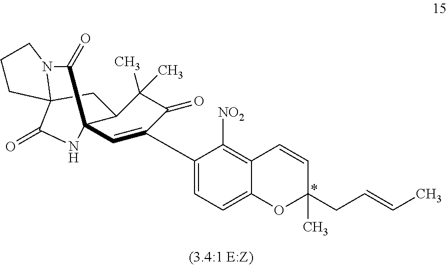

Nitroarene 15. A mixture of tris(dibenzylideneacetone)dipalladium (11.5 mg, 12.6 μmol, 25.1 μmol Pd) and triphenylarsine (15.4 mg, 50.2 μmol, 2 equiv based on Pd) in N,N-dimethylformamide (500 μL, deoxygenated by bubbling argon gas through the solvent for 1 h before use) was stirred at 23° C. for 30 min. In a separate flask, a suspension of copper iodide (5 mg, 26.3 μmol) in N,N-dimethylformamide (500 μL, deoxygenated by bubbling argon gas through the solvent for 1 h before use) was stirred at 23° C. for 30 min.

A third flask was charged with the vinyl iodide 14 (20 mg, 50 μmol, 1 equiv), the stannane 13 (53 mg, 100 μmol, 2 equiv), and N,N-dimethylformamide (500 μL, deoxygenated by bubbling argon gas through the solvent for 1 h before use). The resulting solution was treated sequentially with the tris(dibenzylideneacetone)dipalladium-triphenylarsine and copper iodide solutions prepared above (100 μL each). The reaction mixture was stirred at 23° C. for 48 h. The product solution was diluted with hexanes-ethyl ether (2:1, 100 mL). The diluted solution was washed successively with water and saturated aqueous sodium chloride solution. The combined aqueous layers were extracted with hexanes-ethyl ether (2:1). The combined organic phases were dried over anhydrous sodium sulfate, the solids were removed by filtration, and the filtrate was concentrated in vacuo. The residue was purified by flash-column chromatography (dichloromethane-methanol, 100:1 to 100:2), affording the nitroarene 15 (a 1:1 mixture of diastereoisomers at C(21), and a 3.4:1 mixture of E- and Z-geometrical isomers, respectively, 21 mg, 81%) as a yellow solid.

R_{f}=0.50 (hexanes-ethyl acetate 1:9). ¹H NMR (500 MHz, CDCl₃, signals for the major diastereoisomers), δ 7.45-7.30 (1H, br m), 7.11 (d, 1H, J=8.3 Hz), 6.95-6.92 (m, 1H), 6.88 (s, 1H), 6.51 (d, 1H, J=10.3 Hz), 5.79 (d, 1H, J=10.3 Hz), 5.61-5.40 (m, 2H), 3.64-3.59 (m, 1H), 3.47-3.45 (m, 1H), 2.80-2.74 (m, 2H), 2.42-2.40 (m, 2H), 2.23-2.19 (m, 1H), 2.07-1.95 (m, 2H), 1.86-1.81 (m, 3H), 1.67-1.60 (m, 2H), 1.45-1.41 (m, 3H), 1.09 (s, 3H), 1.06 (s, 3H). ¹³C NMR (100 MHz, CDCl₃, signals for the major diastereoisomers), δ 199.2, 172.8, 167.5, 154.6, 146.7, 140.4, 137.9, 133.7, 133.7, 131.4, 130.1, 130.0, 124.7, 124.6, 122.5, 119.7, 119.7, 117.7, 117.6, 115.2, 115.2, 79.2, 67.8, 61.1, 51.0, 45.2, 44.6, 44.2, 44.1, 32.5, 29.5, 26.0, 25.9, 24.8, 23.3, 18.5, 18.3. IR (NaCl, thin film), cm⁻¹ 3215 (br), 2973 (w), 2935 (w), 2881 (w), 1686 (s), 1530 (s), 1353 (m). HRMS-ESI (m/z): [M+H]⁺ calcd for C₂₉H₃₂N₃O₆⁺, 518.2291; found, 518.2301.

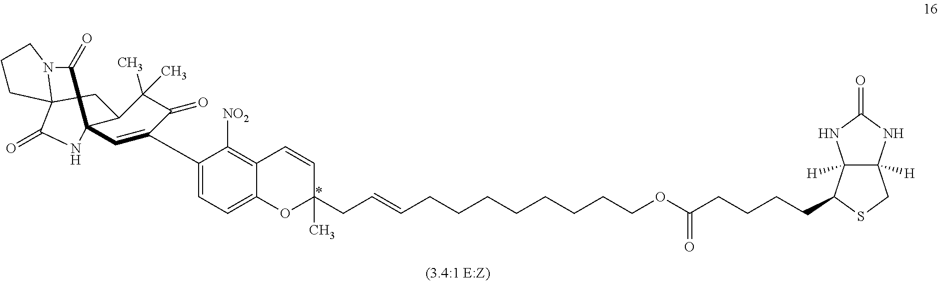

Biotinylated nitroarene 16. A solution of the nitroarene 15 (19 mg, 37 μmol, 1.0 equiv), biotinylated alkene 7 (71 mg, 185 μmol, 5.0 equiv), and Grubbs' second-generation catalyst (3.1 mg, 3.7 μmol, 0.1 equiv) in benzene (20 mL) was stirred at 50° C. for 24h. A second portion of Grubbs' second-generation catalyst (1.6 mg, 1.8 μmol, 0.05 equiv) was added and the solution was stirred at 50° C. for 18 h. The brown reaction mixture was allowed to cool to 23° C. and the cooled solution was concentrated in vacuo. The residue was purified by flash-column chromatography (dichloromethane-methanol, 25:1) to afford the biotinylated derivative 16 (a 1:1 mixture of diastereoisomers at C(21), and a 3.4:1 mixture of E- and Z-geometrical isomers, respectively, 23 mg, 72%) as a yellow film.

R_{f}=0.42 (dichloromethane-methanol 9:1). ¹H NMR (500 MHz, CDCl₃, signals for the major diasteroisomers), δ 9.09 (s, 1H), 7.18-7.10 (m, 1H), 6.98-6.86 (m, 2H), 6.55-6.50 (m, 1H), 6.17 (s, 1H), 5.82-5.76 (m, 1H), 5.55-5.37 (m, 2H), 5.34 (s, 1H), 4.50-4.44 (m, 1H), 4.27-4.22 (m, 1H), 4.08-3.99 (m, 2H), 3.66-3.60 (m, 1H), 3.47 (dt, 1H, J=11.7, 7.3 Hz), 3.13-3.08 (m, 1H), 2.90-2.86 (m, 1H), 2.82-2.75 (m, 2H), 2.71 (d, 1H, J=12.7 Hz), 2.44-2.38 (m, 2H), 2.33-2.29 (m, 2H), 2.23-2.18 (m, 1H), 2.09-1.97 (m, 4H), 1.88-1.82 (m, 2H), 1.72-1.56 (m, 6H), 1.45-1.23 (m, 15H), 1.10-1.06 (m, 6H). ¹³C NMR (100 MHz, CDCl₃, signals for the major diastereoisomers), 199.4, 199.3, 174.0, 173.7, 173.5, 167.8, 167.7, 164.0, 163.9, 154.6, 154.5, 146.7, 146.7, 140.3, 140.0, 138.5, 135.6, 135.4, 133.8, 131.7, 131.6, 123.7, 123.5, 122.9, 122.8, 122.7, 119.9, 119.7, 117.7, 117.6, 115.2, 115.1, 79.4, 79.1, 67.7, 64.8, 64.8, 61.9, 61.9, 61.1, 61.0, 60.6, 60.5, 60.3, 55.6, 55.6, 51.0, 50.9, 46.1, 45.2, 45.1, 44.5, 44.0, 40.8, 34.2, 34.2, 32.7, 32.6, 32.5, 29.9, 29.5, 29.4, 29.4, 29.3, 29.2, 29.1, 29.0, 28.8, 28.6, 28.4, 28.4, 27.6, 26.3, 26.1, 26.1, 25.9, 25.2, 25.1, 24.9, 23.4, 23.2, 18.6, 18.5. IR (NaCl, thin film), cm⁻¹ 3258 (br), 2928 (m), 2855 (w), 1701 (s), 1684 (s), 1529 (m), 1458 (m), 1351 (m), 1267 (w). HRMS-ESI (m/z): [M+H]⁺ calcd for C₄₆H₆₀N₅O₉S⁺, 858.4106; found, 858.4124.

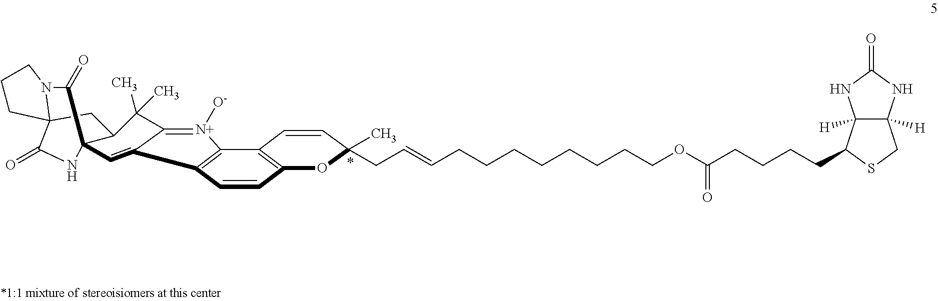

Biotinylated nitrone 5. Aqueous ammonium chloride solution (1 M, 22.4 μL, 22.4 μmol, 3.2 equiv) was added to a solution of the nitroarene 16 (5.6 mg, 7 μmol, 1 equiv) in ethanol (350 μL). Zinc powder (2.3 mg, 35 μmol, 5 equiv) was added and the resulting yellow suspension was stirred 23° C. for 2 hours. The suspension was diluted with ethyl acetate and the diluted suspension was filtered through Celite. The filtrate was washed with saturated aqueous sodium chloride solution, the washed solution was dried over anhydrous sodium sulfate, the solids were removed by filtration, and the filtrate was concentrated in vacuo. The residue was purified by flash-column chromatography (dichloromethane-methanol, 10:1) and further by HPLC (reverse phase, Beckman Coulter Ultrasphere ODS 5 μM, 30% to 100% acetonitrile in water) to afford the nitrone 5 (a 1:1 mixture of diastereoisomers at C(21), 788 μg, 15%) as a yellow solid.

R_{f}=0.39 (dichloromethane-methanol 85:15). ¹H NMR (500 MHz, C₆D₆, signals for the major diastereoisomers), δ 9.22 (br s, 1H), 8.44-8.40 (m, 1H), 6.88-6.85 (m, 1H), 6.77-6.72 (m, 1H), 6.18 (br s, 1H), 5.86 (br s, 1H), 5.57-5.38 (m, 3H), 5.11 (br s, 1H), 4.14-3.99 (m, 3H), 3.73-3.71 (m, 1H), 3.63-3.59 (m, 1H), 3.56-3.53 (m, 1H), 3.41-3.34 (m, 1H), 3.22-3.17 (m, 1H), 2.97-2.85 (m, 1H), 2.72-2.64 (m, 1H), 2.45-1.97 (m, 8H), 1.58-1.08 (m, 31H). IR (NaCl, thin film), cm⁻¹ 3140 (br), 3048 (w), 2931 (w), 2856 (w), 1701 (s), 1404 (m). HRMS-ESI (m/z): [M+H]⁺ calcd for C₄₆H₆₀N₅O₇S⁺, 826.4213; found, 826.4232.

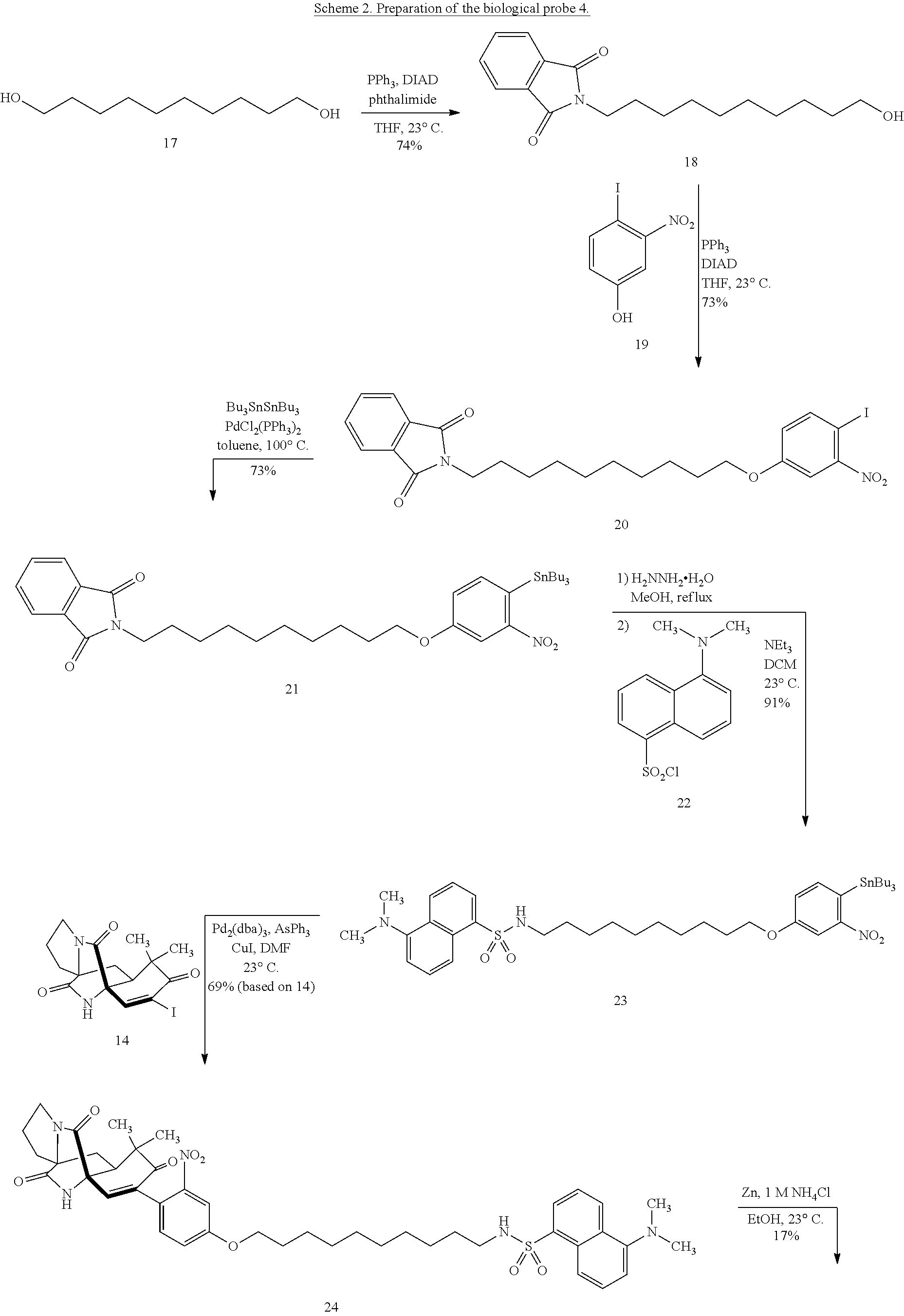

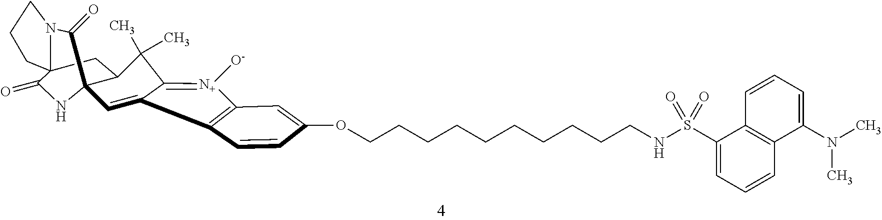

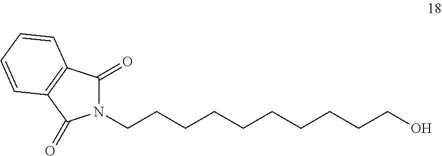

Phthalimide 18. Diisopropyl azodicarboxylate (11.81 mL, 60 mmol, 1.2 equiv) was added slowly to an ice-cooled solution of 1,10-decanediol (17) (26.14 g, 150 mmol, 3.0 equiv), triphenylphosphine (15.73 g, 60 mmol, 1.2 equiv), and phthalimide (7.36 g, 50 mmol, 1.0 equiv) in tetrahydrofuran (125 mL). The resulting yellow solution was stirred at 23° C. for 20 h. The yellow product mixture was concentrated in vacuo and the residue was subjected to flash-column chromatography (hexanes-ethyl acetate, 7:3 to 1:1), affording the phthalimide 18 (11.28 g, 74%) as a white solid.

R_{f}=0.30 (hexanes-ethyl acetate 3:2). ¹H NMR (500 MHz, CDCl₃), δ 7.84 (dd, 2H, J=5.4, 2.9 Hz), 7.71 (dd, 2H, J=5.4, 2.9 Hz), 3.68 (t, 2H, J=7.3 Hz), 3.64 (dd, 2H, J=12.2, 6.4 Hz), 1.68-1.66 (m, 2H), 1.59-1.53 (m, 2H), 1.33-1.27 (m, 12H). ¹³C NMR (125 MHz, CDCl₃), δ 168.7, 134.1, 132.4, 123.4, 63.3, 38.3, 33.0, 29.7, 29.5, 29.3, 28.8, 27.0, 25.9, 22.2. IR (NaCl, thin film), cm⁻¹ 3410 (br), 2927 (m), 2854 (m), 1773 (m), 1705 (s). HRMS-ESI (m/z): [M+H]⁺ calcd for C₁₈H₂₆NO₃⁺, 304.1907; found, 304.1900.

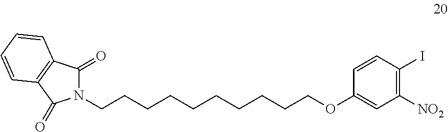

Iodoarene 20. Diisopropyl azodicarboxylate (3.25 mL, 16.5 mmol, 1.1 equiv) was added dropwise to a solution of 4-iodo-3-nitrophenol (19) (3.98 g, 15.0 mmol, 1.0 equiv), the alcohol 18 (5.01 g, 16.5 mmol, 1.1 equiv), and triphenylphosphine (4.33 g, 16.5 mmol, 1.1 equiv) in tetrahydrofuran (37 mL). The orange solution was stirred at 23° C. for 16 hours. The product solution was concentrated in vacuo and the residue was recrystallized from chloroform, furnishing the iodoarene 20 (6.03 g, 73%) as a pale yellow solid.

R_{f}=0.64 (hexanes-ethyl acetate 3:2). ¹H NMR (500 MHz, CDCl₃), δ 7.86-7.83 (m, 3H), 7.71 (dd, 2H, J=5.4, 2.9 Hz), 7.40 (d, 1H, J=2.4 Hz), 6.85 (dd, 1H, J=8.8, 2.9 Hz), 3.97 (t, 2H, J=6.4 Hz), 3.68 (t, 2H, J=7.3 Hz), 1.81-1.76 (m, 2H), 1.69-1.66 (m, 2H), 1.45-1.42 (m, 2H), 1.33-1.25 (m, 10H). ¹³C NMR (100 MHz, CDCl₃), δ 168.7, 159.9, 153.7, 142.2, 134.1, 132.4, 123.4, 121.1, 111.7, 74.3, 69.1, 38.3, 29.6, 29.5, 29.4, 29.3, 29.1, 28.8, 27.0, 26.0. IR (NaCl, thin film), cm⁻¹ 2928 (m), 2854 (m), 1772 (m), 1706 (s). HRMS-ESI (m/z): [M+H]⁺ calcd for C₂₄H₂₈IN₂O₅⁺, 551.1037; found, 551.1039.

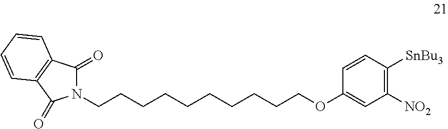

Stannane 21. A solution of the iodoarene 20 (1.10 g, 2.0 mmol, 1 equiv), bis(tributyltin) (1.11 mL, 2.2 mmol, 1.1 equiv), bis(triphenylphosphine)palladium(II) dichloride (14 mg, 20 μmol, 0.01 equiv), and triphenylphosphine (11 mg, 40 μmol, 0.02 equiv) in toluene (20 mL) was stirred at 100° C. for 58 h. The brown suspension was allowed to cool to 23° C. and the cooled mixture was filtered through Celite. The filtrate was concentrated in vacuo and the residue was purified by flash-column chromatography on silica gel (deactivated with 20% triethylamine-ethyl acetate, eluting with hexanes initially, grading to 10% ethyl acetate-hexanes), furnishing the stannane 21 (1.04 g, 73%) as a yellow oil.

R_{f}=0.57 (hexanes-ethyl acetate 4:1). ¹H NMR (500 MHz, C₆D₆), δ 7.86 (d, 1H, J=2.4 Hz), 7.49 (d, 1H, J=8.1 Hz), 7.46 (dd, 2H, J=5.4, 2.9 Hz), 6.99 (dd, 1H, J=8.1, 2.4 Hz), 6.86 (dd, 2H, J=5.4, 2.9 Hz), 3.56 (t, 2H, J=7.1 Hz), 3.47 (t, 2H, J=6.35 Hz), 1.70-1.58 (m, 8H), 1.55-1.49 (m, 2H), 1.37 (sext, 6H, J=7.3 Hz), 1.31-1.17 (m, 18H), 0.90 (t, 9H, J=7.3 Hz). ¹³C NMR (100 MHz, C₆D₆), δ 167.9, 160.5, 155.2, 138.1, 133.3, 132.6, 129.5, 122.8, 121.5, 109.3, 68.2, 37.8, 29.6, 29.6, 29.4, 29.3, 29.3, 29.2, 28.8, 27.6, 27.0, 26.1, 13.8, 11.2. IR (NaCl, thin film), cm⁻¹ 2925 (m), 2854 (m), 1773 (w), 1712 (s), 1603 (w), 1524 (s). HRMS-ESI (m/z): [M+H]⁺ calcd for C₃₆H₅₅N₂O₅Sn⁺, 715.3133; found, 715.3140.

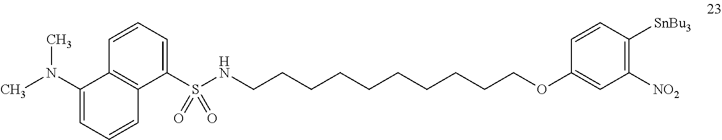

Stannane 23. Hydrazine monohydrate (0.14 mL, 2.89 mmol, 2 equiv) was added to a solution of the stannane 21 (1.03 g, 1.44 mmol, 1 equiv) in methanol (15 mL). The yellow solution was heated to reflux for 2 h. The product solution was allowed to cool to 23° C. and the cooled solution was concentrated in vacuo. The residue was suspended in dichloromethane (ca. 15 mL) and the suspension was dried over anhydrous sodium sulfate. The solids were removed by filtration through Celite and the filtrate was concentrated in vacuo. The resulting yellow oil was dissolved in dichloromethane (5 mL). Dansyl chloride (22) (388 mg, 1.44 mmol, 1 equiv) and triethylamine (0.40 mL, 2.89 mmol, 2 equiv) were added. The yellow solution was stirred at 23° C. for 12 h. The product mixture was concentrated in vacuo and the residue was purified by flash-column chromatography (hexanes-ethyl acetate-triethylamine, 9:1:0.2 to 8:2:0.2), affording the stannane 23 (1.07 g, 91%) as a yellow oil.

R_{f}=0.73 (hexanes-ethyl acetate 3:2). ¹H NMR (500 MHz, C₆D₆), δ 8.68 (d, 1H, J=8.7 Hz), 8.40 (d, 1H, J=8.7 Hz), 8.36 (dd, 1H, 7.3, 1.4 Hz), 7.87 (d, 1H, J=2.3 Hz), 7.50 (d, 1H, J=7.8 Hz), 7.38 (dd, 1H, J=8.7, 7.3 Hz), 7.09 (dd, 1H, J=8.7, 7.3 Hz), 7.00 (dd, 1H, 7.8, 2.3 Hz), 6.84 (d, 1H, J=7.3 Hz), 4.21-4.18 (m, 1H), 3.49 (t, 2H, J=6.4 Hz), 2.64 (q, 2H, J=6.9 Hz), 2.48 (s, 6H), 1.66-1.60 (m, 6H), 1.54 (dt, 2H, J=15.1, 6.4 Hz), 1.37 (sext, 6H, J=7.3 Hz), 1.31-1.21 (m, 8H), 1.19-1.07 (m, 6H), 1.04-0.93 (m, 4H), 0.90 (t, 9H, J=7.3 Hz), 0.87-0.82 (m, 2H). ¹³C NMR (100 MHz, C₆D₆), δ 160.5, 155.2, 152.0, 138.2, 136.4, 130.3, 130.1, 129.7, 129.5, 128.3, 128.2, 123.3, 121.4, 119.9, 115.4, 109.4, 68.2, 45.0, 43.3, 29.7, 29.6, 29.5, 29.5, 29.5, 29.2, 29.1, 27.6, 26.5, 26.1, 13.8, 11.2. IR (NaCl, thin film), cm⁻¹ 3284 (br), 2953 (w), 2925 (m), 2854 (w), 1525 (s), 1330 (s), 1161 (s). HRMS-ESI (m/z): [M+H]⁺ calcd for C₄₀H₆₄N₃O₅SSn⁺, 818.3583; found, 818.3589.

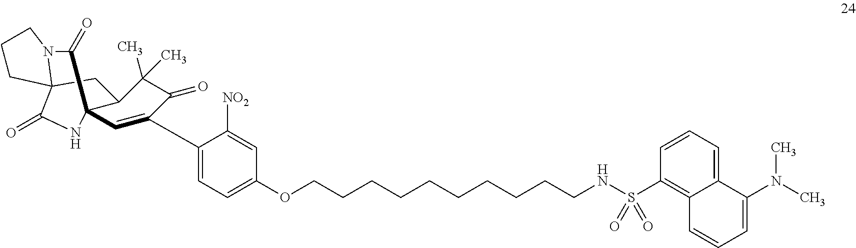

Nitroarene 24. A mixture of tris(dibenzylideneacetone)dipalladium (9 mg, 9.8 μmol, 19.6 μmol Pd) and triphenylarsine (12 mg, 39.2 μmol, 2 equiv based on Pd) in N,N-dimethylformamide (500 μL, deoxygenated by bubbling argon gas through the solvent for 1 h before use) was stirred at 23° C. for 30 min. In a separate flask, a suspension of copper iodide (3.8 mg, 20 μmol) in N,N-dimethylformamide (500 μL, deoxygenated by bubbling argon gas through the solvent for 1 h before use) was stirred at 23° C. for 30 min.

A third flask was charged with the vinyl iodide 14 (8 mg, 20 μmol, 1 equiv), the stannane 23 (33 mg, 40 μmol, 2 equiv), and N,N-dimethylformamide (150 μL, deoxygenated by bubbling argon gas through the solvent for 1 h before use). The resulting solution was treated sequentially with the tris(dibenzylideneacetone)dipalladium-triphenylarsine and copper iodide solutions prepared above (50.0 μL each). The reaction mixture was stirred at 23° C. for 65 h. The product solution was diluted with hexanes-ethyl ether (1:1, 100 mL). The diluted solution was washed with saturated aqueous sodium chloride solution. The aqueous layer was extracted with hexanes-ethyl ether. The combined organic phases were dried over anhydrous sodium sulfate, the solids were removed by filtration, and the filtrate was concentrated in vacuo. The residue was purified by radial chromatography (1-mm rotor, eluting with dichloromethane-triethylamine (100:1) initially, grading to dichloromethane-methanol-triethylamine (100:2:1), affording the nitroarene 24 (11 mg, 69%) as a yellow oil.

R_{f}=0.73 (hexanes-ethyl acetate 3:2). ¹H NMR (500 MHz, CDCl₃), δ 8.53 (d, 1H, J=8.8 Hz), 8.28 (d, 1H, J=8.8 Hz), 8.24 (dd, 1H, J=7.3, 1.5 Hz), 7.60-7.50 (m, 1H), 7.56 (d, 1H, J=7.8 Hz), 7.52 (dd, 1H, J=8.8, 7.3 Hz), 7.31 (br s, 1H), 7.18 (d, 1H, J=7.3 Hz), 7.15-7.11 (m, 1H), 6.91 (br s, 1H), 6.83 (s, 1H), 4.70 (t, 1H, J=5.9 Hz), 4.01 (t, 2H, J=6.6 Hz), 3.66-3.62 (m, 1H), 3.51-3.46 (m, 1H), 2.97-2.78 (m, 4H), 2.89 (s, 6H), 2.24 (dd, 1H, J=13.2, 10.3 Hz), 2.11-1.96 (m, 2H), 1.90-1.84 (m, 2H), 1.81-1.76 (m, 2H), 1.45-1.05 (m, 14H), 1.11 (app s, 6H). ¹³C NMR (100 MHz, CDCl₃), δ 199.2, 172.8, 171.4, 167.6, 160.1, 152.3, 149.2, 141.8, 136.1, 135.0, 133.1, 130.6, 130.1, 129.9, 128.6, 123.4, 123.2, 120.3, 118.9, 115.4, 110.2, 69.0, 67.8, 51.1, 45.6, 45.2, 44.6, 43.5, 32.5, 29.7, 29.6, 29.5, 29.4, 29.3, 29.1, 26.6, 26.0, 24.9, 23.5, 18.7. IR (NaCl, thin film), cm⁻¹ 3245 (br), 2958 (w), 2927 (m), 2854 (w), 1697 (s), 1533 (s). HRMS-ESI (m/z): [M+H]⁺ calcd for C₄₃H₅₄N₅O₈S⁺, 800.3688; found, 800.3655.

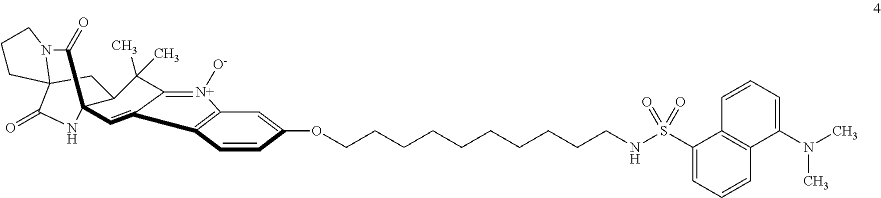

Dansylated nitrone 4. Ammonium chloride solution (1 M, 22 μL, 22 μmol, 3.2 equiv) was added to a solution of the nitroarene 24 (5.6 mg, 7 μmol, 1 equiv) in ethanol (350 μL) and tetrahydrofuran (100 μL). Zinc powder (2.3 mg, 35 μmol, 5 equiv) was added. The resulting pale yellow suspension was stirred at 23° C. for 1 h. The product mixture was diluted with ethyl acetate (9 mL) and the diluted mixture was filtered through Celite. The filtrate was washed with saturated aqueous sodium chloride solution, the washed solution was dried over sodium sulfate, the solids were removed by filtration, and the filtrate was concentrated in vacuo. The residue was subjected to flash-column chromatography (ethyl acetate to ethyl acetate-methanol 20:1). The semi-purified product was purified by HPLC (reverse phase, Beckman Coulter Ultrasphere ODS 5 μM, 30% to 100% acetonitrile in water) to afford the nitrone 4 (929 μg, 17%) as a yellow solid.

R_{f}=0.35 (ethyl acetate-methanol 100:4). ¹H NMR (500 MHz, C₆D₆), δ 8.73 (d, 1H, J=8.7 Hz), 8.40 (d, 1H, J=8.7 Hz), 8.38 (d, 1H, J=7.3 Hz), 7.55 (d, 1H, J=2.3 Hz), 7.38 (t, 1H, J=8.2 Hz), 7.21-7.08 (m, 2H), 6.90 (dd, 1H, J=8.2, 2.3 Hz), 6.85 (d, 1H, J=7.8 Hz), 6.17 (s, 1H), 5.54 (s, 1H), 4.80 (t, 1H, J=6.2 Hz), 3.66 (t, 2H, J=6.2 Hz), 3.23-3.18 (m, 1H), 2.91 (dt, 1H, J=11.0, 7.3 Hz), 2.72-2.62 (m, 3H), 2.49 (s, 6H), 1.99 (dd, 1H, J=10.1, 6.4 Hz), 1.60 (s, 3H), 1.59-1.53 (m, 2H), 1.46-1.39 (m, 2H), 1.31-0.97 (m, 13H), 1.24 (s, 3H), 0.91-0.81 (m, 4H). IR (NaCl, thin film), cm⁻¹ 3300 (br), 2926 (m), 2872 (w), 1697 (s). HRMS-ESI (m/z): [M+H]⁺ calcd for C₄₃H₅₄N₅O₆S⁺, 768.3789; found, 768.3780.

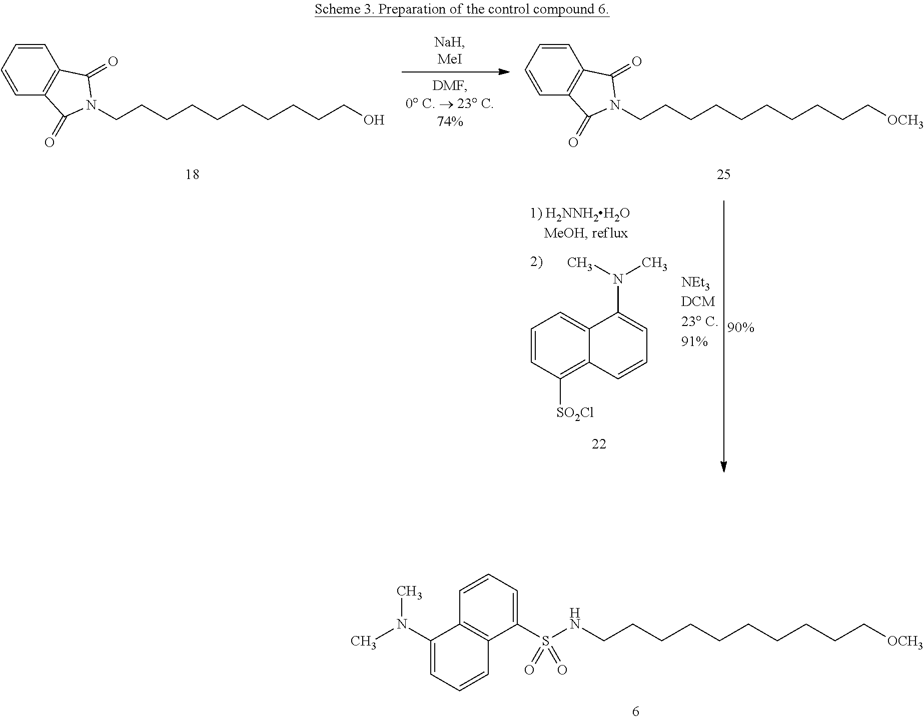

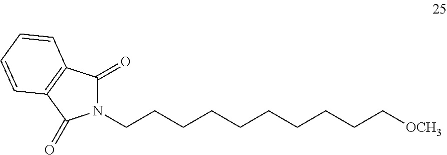

Phthalimide 25.60% Sodium hydride in mineral oil (360 mg, 9 mmol, 1.5 equiv) was added in one portion to an ice-cooled solution of the alcohol 18 (1.82 g, 6 mmol, 1.0 equiv) in N,N-dimethylformamide (20 mL) (gas evolution). The mixture was stirred at 0° C. for 15 min. Methyl iodide (0.56 mL, 9 mmol, 1.5 equiv) was added dropwise. The cooling bath was removed, the reaction mixture was allowed to warm to 23° C., and the mixture was stirred at 23° C. for 20 h. The product mixture was poured on water and ice (160 mL). The resulting mixture was extracted three times with hexane-ethyl ether (2:1). The combined organic phases were washed with saturated aqueous sodium chloride solution, the washed solution was dried over sodium sulfate, the solids were removed by filtration, and the filtrate was concentrated in vacuo. The residue was purified by flash-column chromatography (hexanes-ethyl acetate, 100:20), furnishing the phthalimide 25 (1.41 g, 74%) as a white solid.

R_{f}=0.71 (hexanes-ethyl acetate). ¹H NMR (500 MHz, CDCl₃), δ 7.84 (dd, 2H, J=5.37, 2.93 Hz), 7.71 (dd, 2H, J=5.37, 2.93 Hz), 3.67 (t, 2H, J=7.3 Hz), 3.35 (t, 2H, J=6.8 Hz), 3.32 (s, 3H), 1.66 (dt, 2H, J=14.2, 7.3 Hz), 1.59-1.52 (m, 2H), 1.32-1.27 (m, 12H). ¹³C NMR (100 MHz, CDCl₃), δ 168.7, 134.1, 132.4, 123.4, 73.2, 58.8, 38.3, 29.9, 29.7, 29.7, 29.6, 29.4, 28.8, 27.1, 26.3. IR (NaCl, thin film), cm⁻¹ 2928 (m), 2855 (m), 1773 (m), 1708 (s). HRMS-ESI (m/z): [M+H]⁺ calcd for C₁₉H₂₈NO₃⁺, 318.2069; found, 318.2059.

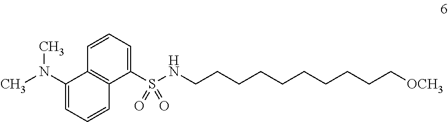

Dansylated derivative 6. Hydrazine monohydrate (0.22 mL, 4.5 mmol, 2 equiv) was added to a solution of the phthalimide 25 (714 mg, 2.25 mmol, 1 equiv) in methanol (20 mL). The clear solution was heated to reflux for 2 h. The product solution was allowed to cool to 23° C. and the cooled solution was concentrated in vacuo. The residue was suspended in dichloromethane (ca. 20 mL), the suspension was dried over anhydrous sodium sulfate, the solids were removed by filtration through Celite, and the filtrate was concentrated in vacuo. The residue was dissolved in dichloromethane (10 mL). Dansyl chloride (22) (607 mg, 2.25 mmol, 1 equiv) and triethylamine (0.63 mL, 4.5 mmol, 2 equiv) were added. The yellow solution was stirred at 23° C. for 20 h. The product solution was concentrated in vacuo and the residue was purified by flash-column chromatography (hexanes-ethyl acetate-triethylamine, 100:10:2 to 100:20:2), affording the dansylated control 6 (852 mg, 2.03 mmol, 90%) as a yellow oil.

R_{f}=0.60 (hexanes-ethyl acetate 3:2). ¹H NMR (500 MHz, CDCl₃), δ 8.54 (d, 1H, J=8.8 Hz), 8.28 (d, 1H, J=8.8 Hz), 8.25 (dd, 1H, J=7.3, 1.0 Hz), 7.57 (dd, 1H, J=8.8, 7.3 Hz), 7.53 (dd, 1H, J=8.8, 7.3 Hz), 7.19 (d, 1H, J=7.3 Hz), 4.53 (t, 1H, J=6.3 Hz), 3.35 (t, 2H, J=6.8 Hz), 3.33 (s, 3H), 2.89 (s, 6H), 2.89-2.86 (m, 2H), 1.54 (dt, 2H, J=14.6 6.8 Hz), 1.37-1.32 (m, 2H), 1.30-1.09 (m, 12H). ¹³C NMR (100 MHz, CDCl₃), δ 152.3, 134.9, 130.6, 130.1, 129.9, 129.9, 128.6, 123.4, 118.9, 115.4, 73.2, 58.8, 45.6, 43.6, 29.9, 29.7, 29.6, 29.5, 29.1, 26.6, 26.3. IR (NaCl, thin film), cm⁻¹ 3301 (br), 2928 (m), 2854 (m), 1589 (w), 1576 (w), 1457 (m), 1321 (s), 1160 (s). HRMS-ESI (m/z): [M+H]⁺ calcd for C₂₃H₃₇N₂O₃S⁺, 421.2525; found, 421.2538.

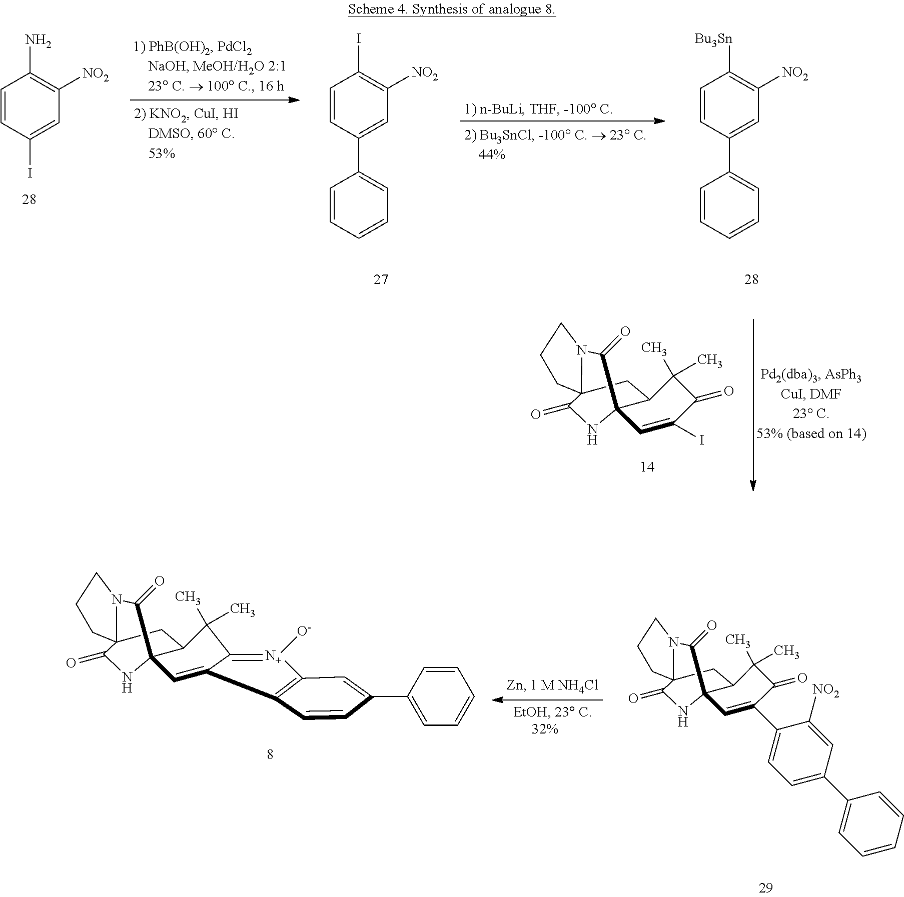

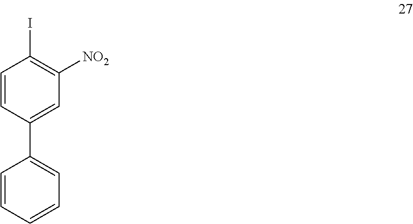

Iodoarene 27. A mixture of 4-iodo-2-nitroaniline (26) (1.06 g, 4.0 mmol, 1 equiv), phenylboronic acid (536 mg, 4.4 mmol, 1.1 equiv), palladium chloride (35 mg, 0.2 mmol, 0.05 equiv), and sodium hydroxide (640 mg, 16 mmol, 4 equiv) in methanol-water (2:1, 15 mL) was stirred at 23° C. for 19 h and further at 100° C. for 3 hours. The mixture was allowed to cool to 23° C. and the cooled mixture was concentrated in vacuo. The residue was neutralized with 5% hydrochloric acid solution. The resulting solution was extracted four times with ethyl acetate. The combined organic phases were dried over anhydrous sodium sulfate, the solids were removed by filtration, and the filtrate was concentrated in vacuo. The resulting brown solid, potassium nitrite (857 mg, 4.0 mmol, 1 equiv), and copper iodide (762 mg, 4.0 mmol, 1 equiv) were suspended in dimethylsulfoxide and the mixture was heated to 60° C. A solution of 55% hydroiodic acid (5 mL) in dimethylsulfoxide was added dropwise to the warmed reaction mixture. The resulting dark red solution was stirred at 60° C. for 30 min. The solution was allowed to cool to 23° C. and the cooled reaction mixture was poured onto a mixture of potassium carbonate (5 g) in ice-water (100 mL). The mixture was extracted three times with ethyl ether. The combined organic phases were washed successively with water and saturated aqueous sodium chloride solution. The washed solution was dried over anhydrous sodium sulfate, the solids were removed by filtration, and the filtrate was concentrated in vacuo. The residue was purified by flash-column chromatography (hexanes-dichloromethane, 9:1 to 8:2), affording the iodoarene 27 (684 mg, 53%) as a yellow solid.

R_{f}=0.32 (hexanes-acetone 100:4). ¹H NMR (500 MHz, CDCl₃), δ 8.10-8.07 (m, 2H), 7.60-7.58 (m, 2H), 7.51-7.42 (m, 4H). ¹³C NMR (100 MHz, CDCl₃), δ 143.0, 142.4, 137.9, 132.0, 129.5, 129.1, 127.1, 124.1, 84.6. IR (NaCl, thin film), cm⁻¹ 3086 (w), 3064 (w), 2871 (w), 1540 (s), 1507 (m), 1465 (m), 1345 (m) 1025 (m), 1019 (m).

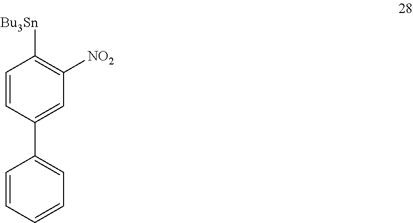

Stannane 28. n-Butyllithium in hexanes (2.48 M, 0.42 mL, 1.05 mmol, 1.05 equiv) and tributyltin chloride (0.28 mL, 1.05 mmol, 1.05 equiv) were added in sequence to a solution of iodoarene 27 (325 mg, 1.0 mmol, 1 equiv) in tetrahydrofuran (10 mL) cooled to −100° C. The cooling bath was removed and the brown solution was allowed to warm to 23° C. over 45 min. The solution was diluted with hexanes-ethyl ether (2:1) and the diluted solution was washed successively with water and saturated aqueous sodium chloride solution. The washed solution was dried over anhydrous sodium sulfate, the solids were removed by filtration, and the filtrate was concentrated in vacuo. The residue was purified by flash-column chromatography on silica gel (deactivated with 20% triethylamine-ethyl acetate, eluting with hexanes-ethyl acetate 100:2), furnishing the stannane 28 (213 mg, 44%) as a yellow oil.

R_{f}=0.75 (hexanes-acetone 100:4). ¹H NMR (500 MHz, C₆D₆), δ 8.49 (d, 1H, J=1.5 Hz), 7.61 (d, 1H, J=7.8 Hz), 7.45 (dd, 1H, J=7.8, 1.5 Hz), 7.28-7.26 (m, 2H), 7.17-7.11 (m, 3H), 1.67-1.60 (m, 6H), 1.38 (sext, 6H, J=7.3 Hz), 1.28-1.24 (m, 6H), 0.91 (t, 9H, J=7.3 Hz). ¹³C NMR (125 MHz, C₆D₆), δ 154.8, 142.8, 138.9, 138.1, 131.7, 129.1, 128.3, 128.2, 127.1, 122.5, 29.4, 27.6, 13.8, 11.3. IR (NaCl, thin film), cm⁻¹ 2956 (m), 2922 (m), 2852 (w), 1534 (s), 1343 (m).

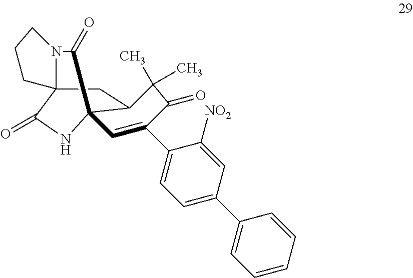

Nitroarene 29. A mixture of tris(dibenzylideneacetone)dipalladium (9 mg, 9.8 μmol, 19.6 μmol Pd) and triphenylarsine (12 mg, 39.2 μmol, 2 equiv based on Pd) in N,N-dimethylformamide (500 μL, deoxygenated by bubbling argon gas through the solvent for 1 h before use) was stirred at 23° C. for 30 min. In a separate flask, a suspension of copper iodide (3.8 mg, 20 μmol) in N,N-dimethylformamide (500 μL, deoxygenated by bubbling argon gas through the solvent for 1 h before use) was stirred at 23° C. for 30 min.

A third flask was charged with vinyl iodide 14 (8 mg, 20 μmol, 1 equiv), stannane 28 (20 mg, 40 μmol, 2 equiv), and N,N-dimethylformamide (150 μL, deoxygenated by bubbling argon gas through the solvent for 1 h before use). The resulting solution was treated sequentially with the tris(dibenzylideneacetone)dipalladium-triphenylarsine and copper iodide solutions prepared above (50.0 μL each). The reaction mixture was stirred at 23° C. for 61 h. The product solution was diluted with hexanes-ethyl ether (2:1, 100 mL). The diluted solution was washed with saturated aqueous sodium chloride solution. The aqueous layer was extracted with hexanes-ethyl ether (2:1). The combined organic phases were dried over anhydrous sodium sulfate, the solids were removed by filtration, and the filtrate was concentrated in vacuo. The residue was purified by radial chromatography (1-mm rotor, eluting with dichloromethane-methanol, 100:1), affording the nitroarene 29 (5 mg, 53%) as a pale yellow solid.

R_{f}=0.35 (hexanes-ethyl acetate 1:9). ¹H NMR (500 MHz, CDCl₃), δ 8.31 (s, 1H), 7.86 (d, 1H, J=7.8 Hz), 7.62 (d, 2H, J=7.3 Hz), 7.52-7.43 (m, 4H), 6.92 (s, 1H), 6.77 (br s, 1H), 3.69-3.64 (m, 1H), 3.50 (dt, 1H, J=11.2, 7.6 Hz), 2.96-2.80 (m, 2H), 2.29 (dd, 1H, J=13.2, 10.3 Hz), 2.14-1.99 (m, 2H), 1.93-1.86 (m, 2H), 1.16 (s, 6H). ¹³C NMR (100 MHz, CDCl₃), δ 199.0, 172.7, 167.5, 149.0, 143.4, 142.0, 138.3, 136.5, 132.7, 132.1, 129.9, 129.4, 129.0, 127.3, 123.1, 67.8, 61.1, 51.1, 45.2, 44.6, 32.5, 29.6, 24.9, 23.5, 18.8. IR (NaCl, thin film), cm⁻¹ 2921 (w), 1686 (s), 1532 (m), 1352 (w). HRMS-ESI (m/z): [M+H]⁺ calcd for C₂₇H₂₆N₃O₅⁺, 472.1867; found, 472.1850.

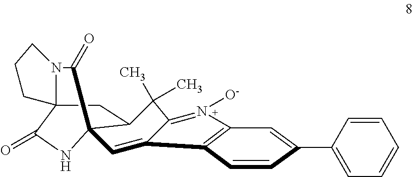

Nitrone 8. Ammonium chloride solution (1 M, 15 μL, 15 μmol, 2.2 equiv) was added to a solution of nitroarene 29 (3.3 mg, 7 μmol, 1 equiv) in ethanol (350 μL). Zinc powder (2.3 mg, 35 μmol, 5 equiv) was added. The resulting pale yellow suspension was stirred at 23° C. for 15 min. The product mixture was diluted with ethyl acetate (9 mL) and the diluted mixture was filtered through Celite. The filtrate was washed with saturated aqueous sodium chloride solution, the washed solution was dried over sodium sulfate, the solids were removed by filtration, and the filtrate was concentrated in vacuo. The residue was filtered through a plug of silica gel, eluting with dichloromethane-acetone (2:1). The filtrate was concentrated in vacuo and the residue was purified by radial chromatography (1-mm rotor, eluting with dichloromethane-methanol 100:1 initially, grading to dichloromethane-methanol 100:3), affording the nitrone 8 (970 μg, 32%), as a yellow solid.

R_{f}=0.40 (dichloromethane-methanol 100:6). ¹H NMR (500 MHz, C₆D₆), δ 8.17 (s, 1H), 7.40-7.36 (m, 4H), 7.21-7.03 (m, 3H), 6.17 (s, 1H), 5.36 (br s, 1H), 3.19-3.15 (m, 1H), 2.89 (dt, 1H, J=11.2, 7.3 Hz), 2.65-2.60 (m, 1H), 1.91 (dd, 1H, J=9.8, 6.8 Hz), 1.57 (s, 3H), 1.43-1.08 (m, 5H), 1.23 (s, 3H). IR (NaCl, thin film), cm⁻¹ 3215 (w), 2925 (w), 1702 (s), 1686 (s). HRMS-ESI (m/z): [M+H]⁺ calcd for C₂₇H₂₆N₃O₃⁺, 440.1969; found, 440.1962.

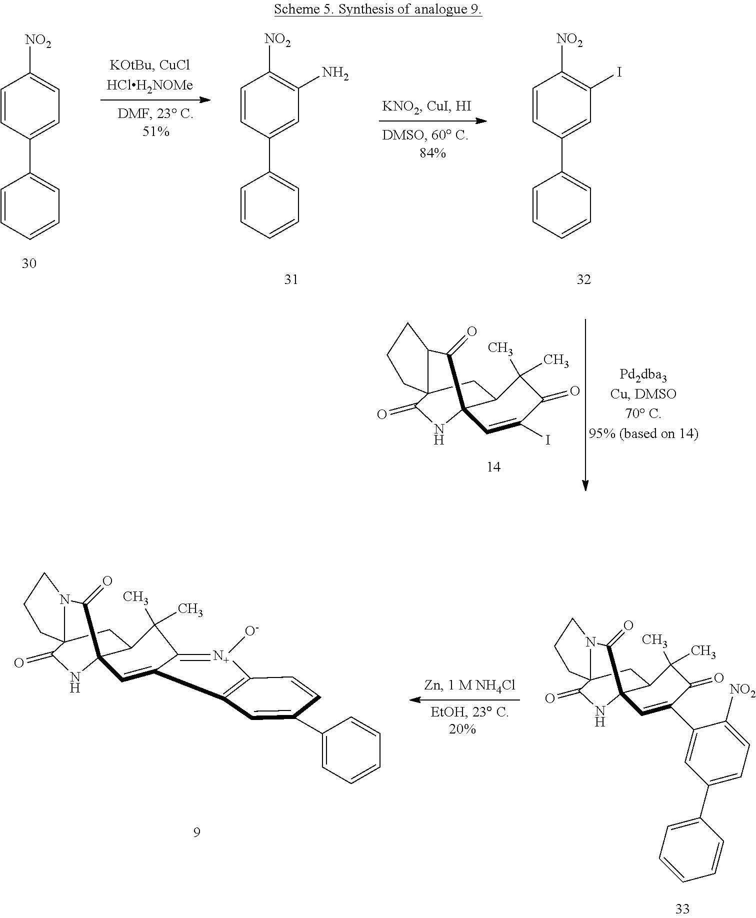

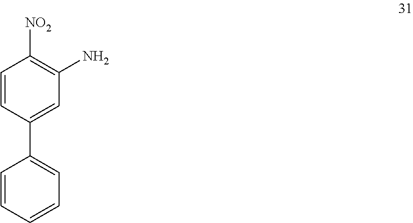

Nitroaniline 31. A solution of the nitroarene 30 (1.54 g, 7.73 mmol, 1.0 equiv) and methoxylamine hydrochloride (807 mg, 9.66 mmol, 1.25 equiv) in dimethylformamide (12 mL) was added over 5 min to a solution of potassium tert-butoxide (3.69 g, 32.85 mmol, 4.25 equiv) and copper chloride (77 mg, 0.1 mmol, 0.1 equiv) in dimethylformamide (27 mL). The resulting dark red solution was stirred at 23° C. for 1.5 h. The product solution was diluted with saturated ammonium chloride solution and the diluted solution was extracted three times with dichloromethane. The combined organic phases were dried over sodium sulfate, the solids were removed by filtration, and the filtrate was concentrated in vacuo. The residue was purified by recrystallization (hexanes-ethyl acetate), affording the nitroaniline 31 (853 mg, 51%) as a yellow solid.

R_{f}=0.35 (hexanes-ethyl acetate 8:2). ¹H NMR (500 MHz, CDCl₃), δ 8.19 (d, 1H, J=8.8 Hz), 7.59-7.57 (m, 2H), 7.49-7.41 (m, 3H), 6.99 (d, 1H, J=2.0 Hz), 6.94 (dd, 1H, J=8.8, 2.0 Hz), 6.15 (br s, 2H). ¹³C NMR (100 MHz, CDCl₃), δ 148.8, 145.1, 139.2, 131.7, 129.2, 129.1, 127.4, 127.1, 116.8, 116.7. IR (NaCl, thin film), cm⁻¹ 3487 (m), 3369 (m), 3179 (w), 3066 (w), 1620 (s), 1572 (s), 1483 (s), 1444 (s), 1416 (m), 1331 (s), 1282 (s), 1231 (s). HRMS-ESI (m/z): [M+H]⁺ calcd for C₁₂H₁₁N₂O₂⁺, 215.0815; found, 215.0811.

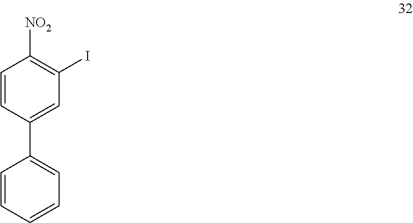

Iodoarene 32. A solution of 55% hydroiodic acid (4.93 mL) in dimethylsulfoxide (16 mL) was added dropwise to a mixture of the nitroaniline 31 (840 mg, 3.92 mmol, 1 equiv), potassium nitrite (734 mg, 8.62 mmol, 2.2 equiv), and copper iodide (747 mg, 3.92 mmol, 1 equiv) in dimethylsulfoxide (20 mL) at 60° C. The dark red mixture was stirred at 60° C. for 30 min. The mixture was allowed to cool to 23° C. and the cooled mixture was poured onto potassium carbonate (5 g) in ice-water (100 mL). The mixture was extracted three times with ethyl ether. The combined organic phases were washed successively with water and saturated aqueous sodium chloride solution. The washed solution was dried over anhydrous sodium sulfate, the solids were removed by filtration, and the filtrate was concentrated in vacuo. The residue was purified by flash-column chromatography (hexanes-dichloromethane, 9:1 to 8:2), affording the iodoarene 32 (1.07 g, 84%) as a pale yellow solid.

R_{f}=0.68 (hexanes-ethyl acetate 8:2). ¹H NMR (500 MHz, CDCl₃), δ 8.26 (d, 1H, J=1.8 Hz), 7.98 (d, 1H, J=8.5 Hz), 7.68 (dd, 1H, J=8.5, 1.8 Hz), 7.59-7.57 (m, 2H), 7.52-7.44 (m, 3H). ¹³C NMR (100 MHz, CDCl₃), δ 146.9, 140.7, 137.6, 129.5, 129.4, 127.7, 127.6, 126.2, 87.3. IR (NaCl, thin film), cm⁻¹ 3061 (w), 3031 (w), 1583 (m), 1566 (m), 1522 (s), 1345 (m). HRMS-ESI (m/z): [M+H]⁺ calcd for C₂₃H₃₇N₂O₃S⁺, ; found, .

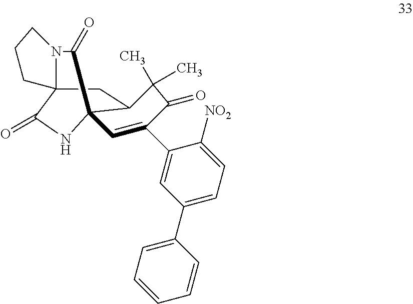

Nitroarene 33. A mixture of the vinyl iodide 14 (8 mg, 20 mmol, 1.0 equiv), the aryl iodide 32 (16.3 mg, 50 mmol, 2.5 equiv), tris(dibenzylideneacetone)dipalladium (1.8 mg, 2 mmol, 0.1 equiv), and copper (6.4 mg, 100 mmol, 5.0 equiv) in dimethylsulfoxide (200 mL) was stirred at 70° C. for 4 h. The brown product mixture was allowed to cool to 23° C. and the cooled mixture was diluted with dichloromethane. The diluted mixture was washed with saturated aqueous ammonium solution-water-ammonium hydroxide (4:1:0.5). The layers were separated and the aqueous phase was extracted with dichloromethane. The combined organic phases were dried over sodium sulfate, the solids were removed by filtration, and the filtrate was concentrated in vacuo. The residue was purified by flash-column chromatography (dichloromethane-methanol, 100:1), furnishing the nitroarene 33 (9 mg, 95%) as a pale yellow solid.

R_{f}=0.40 (hexanes-ethyl acetate 1:9). ¹H NMR (500 MHz, CDCl₃), δ 8.20 (d, 1H, J=8.8 Hz), 7.73-7.62 (m, 3H), 7.50-7.44 (m, 4H), 6.91 (s, 1H), 6.90 (br s, 1H), 3.66-3.61 (m, 1H), 3.52-3.47 (m, 1H), 2.95-2.77 (m, 2H), 2.29-2.25 (m, 1H), 2.10-1.99 (m, 2H), 1.93-1.84 (m, 2H), 1.15 (s, 6H). ¹³C NMR (100 MHz, CDCl₃), δ 199.0, 172.9, 167.4, 147.3, 147.2, 142.5, 138.5, 136.5, 132.2, 130.8, 129.4, 129.2, 128.2, 127.7, 125.4, 67.8, 61.2, 50.8, 45.2, 44.6, 32.5, 29.6, 24.8, 23.9, 18.9. IR (NaCl, thin film), cm⁻¹ 2968 (w), 1688 (s), 1520 (m), 1350 (w). HRMS-ESI (m/z): [M+H]⁺ calcd for C₂₇H₂₆N₃O₅⁺, 472.1867; found, 472.1865.

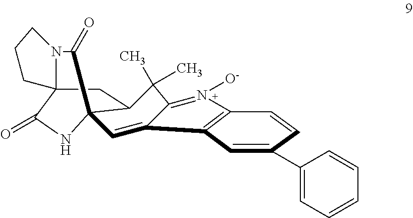

Nitrone 9. Ammonium chloride solution (1 M, 18 μL, 18 μmol, 2.2 equiv) was added to a solution of the nitroarene 33 (3.8 mg, 8 μmol, 1 equiv) in ethanol (400 μL). Zinc powder (2.6 mg, 40 μmol, 5 equiv) was added. The resulting pale yellow suspension was stirred at 23° C. for 30 min. The product mixture was diluted with ethyl acetate (9 mL) and the diluted mixture was filtered through Celite. The filtrate was washed with saturated aqueous sodium chloride solution, the washed solution was dried over sodium sulfate, the solids were removed by filtration, and the filtrate was concentrated in vacuo. The residue was filtered through a plug of silica gel, eluting with dichloromethane-acetone (2:1). The filtrate was concentrated in vacuo and the residue was purified by radial chromatography (1-mm rotor, eluting with dichloromethane-methanol 100:1 initially, grading to dichloromethane-methanol 100:3), affording the nitrone 9 (702 μg, 20%) as a yellow solid.

R_{f}=0.35 (dichloromethane-methanol 100:6). ¹H NMR (500 MHz, C₆D₆), δ 7.81 (d, 1H, J=8.3 Hz), 7.49 (d, 1H, J=1.5 Hz), 7.34 (d, 2H, J=7.3 Hz), 7.31-7.17 (m, 2H), 7.23 (d, 2H, J=7.3 Hz), 6.10 (s, 1H), 5.49 (1H, br s), 3.21-3.16 (m, 1H), 2.89 (dt, 1H, J=11.2, 7.3 Hz), 2.66-2.61 (m, 1H), 1.97 (dd, 1H, J=10.3, 6.8 Hz), 1.58 (s, 3H), 1.45-1.13 (m, 5H), 1.23 (s, 3H). IR (NaCl, thin film), cm⁻¹ 3226 (w), 2961 (w), 2928 (w), 1701 (s), 1689 (s). HRMS-ESI (m/z): [M+H]⁺ calcd for C₂₇H₂₆N₃O₃⁺, 440.1969; found, 440.1969.

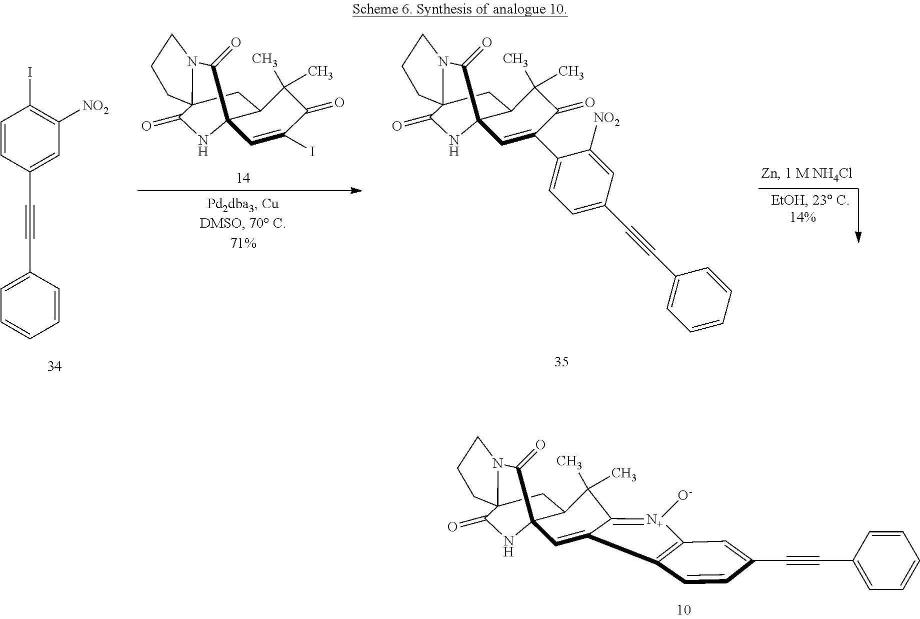

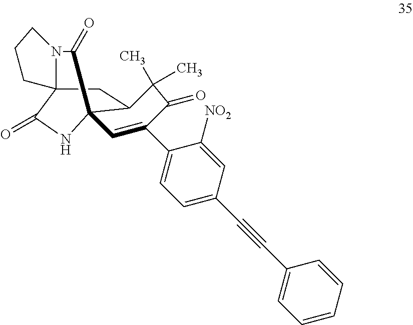

Nitroarene 35. A mixture of the vinyl iodide 14 (8 mg, 20 mmol, 1.0 equiv), the iodoarene 34 (17.5 mg, 50 mmol, 2.5 equiv), tris(dibenzylideneacetone)dipalladium (1.8 mg, 2 mmol, 0.1 equiv), and copper (6.4 mg, 100 mmol, 5.0 equiv) in dimethylsulfoxide (200 mL) was stirred at 70° C. for 5 h. The brown product mixture was allowed to cool to 23° C. and the cooled mixture was diluted with dichloromethane. The diluted mixture was washed with saturated aqueous ammonium solution-water-ammonium hydroxide (4:1:0.5). The layers were separated and the aqueous phase was extracted with dichloromethane. The combined organic phases were dried over sodium sulfate, the solids were removed by filtration, and the filtrate was concentrated in vacuo. The residue was purified by flash-column chromatography (dichloromethane-methanol, 100:1), furnishing the nitroarene 35 (7 mg, 71%) as a pale yellow solid.

R_{f}=0.45 (hexanes-ethyl acetate 1:9). ¹H NMR (500 MHz, CDCl₃), δ 8.24 (d, 1H, J=1.4 Hz), 7.77 (dd, 1H, J=7.8, 1.4 Hz), 7.57-7.55 (m, 2H), 7.44-7.37 (m, 4H), 6.90 (s, 1H), 6.81 (br s, 1H), 3.68-3.63 (m, 1H), 3.50 (dt, 1H, J=11.4, 7.6 Hz), 2.85-2.80 (m, 2H), 2.27 (dd, 1H, J=13.3, 10.0 Hz), 2.13-1.99 (m, 2H), 1.92-1.86 (m, 2H), 1.14 (s, 6H). ¹³C NMR (100 MHz, CDCl₃), δ 198.7, 172.7, 167.4, 148.6, 141.7, 136.9, 136.3, 132.4, 132.1, 130.8, 129.4, 128.7, 127.4, 125.8, 122.3, 93.0, 86.8, 67.8, 61.1 51.0, 45.2, 44.6, 32.4, 29.6, 24.9, 23.5, 18.8. IR (NaCl, thin film), cm⁻¹ 2924 (w), 1688 (s), 1531 (m), 1353 (m). HRMS-ESI (m/z): [M+H]⁺ calcd for C₂₉H₂₆N₃O₅⁺, 496.1867; found, 496.1872.

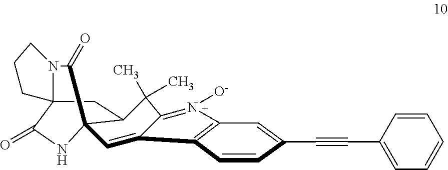

Nitrone 10. Ammonium chloride solution (1 M, 18 μL, 18 μmol, 2.2 equiv) was added to a solution of nitroarene 35 (4.0 mg, 8 μmol, 1 equiv) in ethanol (400 μL). Zinc powder (2.6 mg, 40 μmol, 5 equiv) was added. The resulting pale yellow suspension was stirred at 23° C. for 1 h. The product mixture was diluted with ethyl acetate (9 mL) and the diluted mixture was filtered through Celite. The filtrate was washed with saturated aqueous sodium chloride solution, the washed solution was dried over sodium sulfate, the solids were removed by filtration, and the filtrate was concentrated in vacuo. The residue was filtered through a plug of silica gel, eluting with dichloromethane-acetone (2:1). The filtrate was concentrated in vacuo and the residue was purified by radial chromatography (1-mm rotor, eluting with dichloromethane-methanol 100:1 initially, grading to dichloromethane-methanol 100:3), affording the nitrone 10 (489 μg, 14%) as a yellow solid.

R_{f}=0.31 (dichloromethane-methanol 100:6). ¹H NMR (500 MHz, C₆D₆), δ 8.20 (s, 1H), 7.50-7.49 (m, 2H), 7.41-7.40 (m, 1H), 7.22-7.00 (m, 4H), 6.08 (s, 1H), 5.24 (br s, 1H), 3.17-3.12 (m, 1H), 2.87 (dt, 1H, J=11.2, 7.3 Hz), 2.63-2.56 (m, 1H), 1.84 (dd, 1H, J=10.3, 6.3 Hz), 1.55 (s, 3H), 1.42-1.11 (m, 5H), 1.16 (s, 3H). IR (NaCl, thin film), cm⁻¹ 2954 (w), 2913 (w), 2851 (w), 1692 (s), 1260 (m). HRMS-ESI (m/z): [M+H]⁺ calcd for C₂₉H₂₆N₃O₃⁺, 464.1969; found, 464.1992.

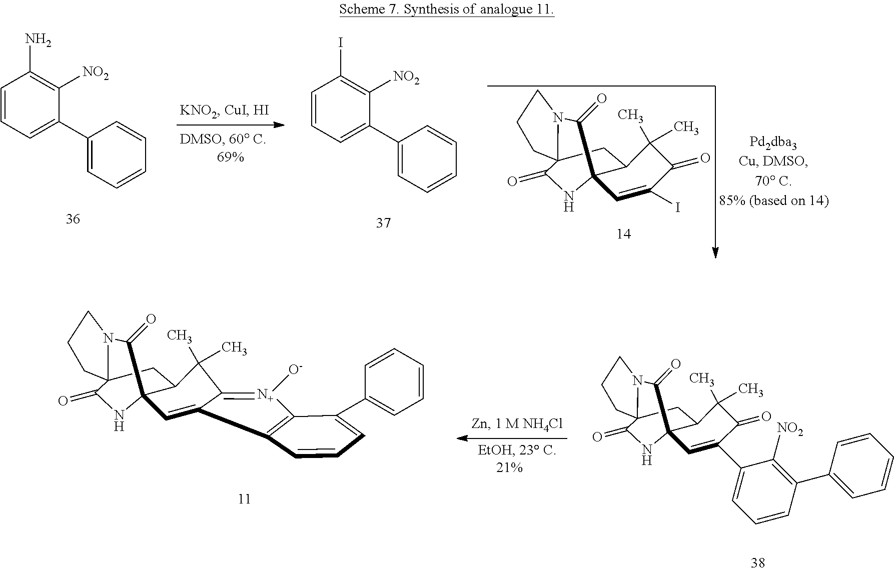

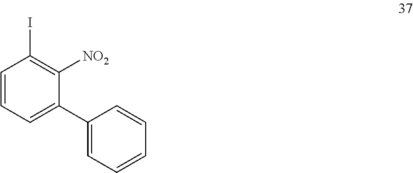

Iodoarene 37. A solution of 55% hydroiodic acid (3.89 mL) in dimethylsulfoxide (12 mL) was added dropwise to a mixture of the nitroaniline 36 (666 mg, 3.11 mmol, 1 equiv), potassium nitrite (582 mg, 6.84 mmol, 2.2 equiv), and copper iodide (592 mg, 3.11 mmol, 1 equiv) in dimethylsulfoxide (15 mL) at 60° C. The dark red mixture was stirred at 60° C. for 30 min. The mixture was allowed to cool to 23° C. and the cooled mixture was poured onto potassium carbonate (5 g) in ice-water (100 mL). The mixture was extracted three times with ethyl ether. The combined organic phases were washed successively with water and saturated aqueous sodium chloride solution. The washed solution was dried over anhydrous sodium sulfate, the solids were removed by filtration, and the filtrate was concentrated in vacuo. The residue was purified by flash-column chromatography (hexanes-dichloromethane, 9:1 to 8:2), affording the iodoarene 37 (693 mg, 69%) as a white solid.

R_{f}=0.46 (hexanes-ethyl acetate 8:2). ¹H NMR (500 MHz, CDCl₃), δ 7.90-7.88 (m, 1H), 7.44-7.40 (m, 3H), 7.35-7.32 (m, 2H), 7.24 (t, 2H, J=7.8 Hz). ¹³C NMR (100 MHz, CDCl₃), δ 139.3, 136.1, 136.0, 131.4, 131.3, 129.2, 129.1, 128.2, 85.8. IR (NaCl, thin film), cm⁻¹ 3084 (w), 3070 (m), 3032 (w), 1522 (s), 1367 (s). HRMS-ESI (m/z): [M+H]⁺ calcd for C₁₂H₈IKNO₂⁺, 363.9231; found, 363.9229.

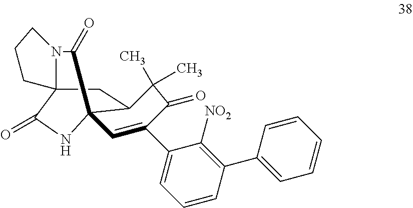

Nitroarene 38. A mixture of the vinyl iodide 14 (8 mg, 20 μmol, 1.0 equiv), the iodoarene 37 (16.3 mg, 50 μmol, 2.5 equiv), tris(dibenzylideneacetone)dipalladium (1.8 mg, 2 μmol, 0.1 equiv), and copper (6.4 mg, 100 μmol, 5.0 equiv) in dimethylsulfoxide (200 μL) was stirred at 70° C. for 5 h. The brown product mixture was allowed to cool to 23° C. and the cooled mixture was diluted with dichloromethane. The diluted mixture was washed with saturated aqueous ammonium solution-water-ammonium hydroxide (4:1:0.5). The layers were separated and the aqueous phase was extracted with dichloromethane. The combined organic phases were dried over sodium sulfate, the solids were removed by filtration, and the filtrate was concentrated in vacuo. The residue was purified by flash-column chromatography (dichloromethane-methanol, 100:1), furnishing the nitroarene 38 (8 mg, 85%) as a pale yellow solid.

R_{f}=0.35 (hexanes-ethyl acetate 1:9). ¹H NMR (500 MHz, CDCl₃), δ 7.59-7.55 (m, 1H), 7.45 (dd, 1H, J=7.8, 1.4 Hz), 7.43-7.35 (m, 4H), 7.34-7.32 (m, 2H), 6.90 (s, 1H), 6.67-6.56 (m, 1H), 3.67-3.62 (m, 1H), 3.49 (dt, 1H, J=11.4, 7.3 Hz), 2.85-2.78 (m, 2H), 2.25 (dd, 1H, J=13.3, 10.5 Hz), 2.12-1.98 (m, 2H), 1.90-1.84 (m, 2H), 1.14 (s, 3H), 1.11 (s, 3H). ¹³C NMR (125 MHz, CDCl₃), δ 199.1, 172.6, 167.1, 149.5, 139.9, 139.0, 137.0, 135.7, 132.1, 130.8, 130.6, 129.8, 128.9, 128.7, 128.2, 67.8, 61.1, 51.1, 45.3, 44.6, 32.5, 29.6, 24.9, 23.3, 18.7. IR (NaCl, thin film), cm⁻¹ 2925 (m), 1686 (s), 1533 (m), 1358 (m). HRMS-ESI (m/z): [M+H]⁺ calcd for C₂₇H₂₆N₃O₅⁺, 472.1867; found, 472.1861.

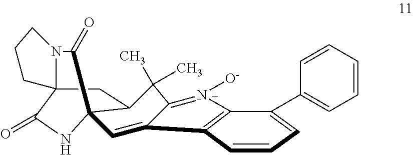

Nitrone 11. Ammonium chloride solution (1 M, 18 μL, 18 μmol, 2.2 equiv) was added to a solution of nitroarene 38 (3.8 mg, 8 μmol, 1 equiv) in ethanol (400 μL). Zinc powder (2.6 mg, 40 μmol, 5 equiv) was added. The resulting pale yellow suspension was stirred at 23° C. for 2 h. The product mixture was diluted with ethyl acetate (9 mL) and the diluted mixture was filtered through Celite. The filtrate was washed with saturated aqueous sodium chloride solution, the washed solution was dried over sodium sulfate, the solids were removed by filtration, and the filtrate was concentrated in vacuo. The residue was subjected to flash-column chromatography (dichloromethane-ethyl acetate, 4:1 to 5:3), giving the nitrone 11 (731 μg, 21%) as a yellow solid.

R_{f}=0.39 (dichloromethane-methanol 100:6). ¹H NMR (500 MHz, C₆D₆), δ 7.56 (d, 2H, J=6.8 Hz), 7.28-7.00 (m, 6H), 6.08 (s, 1H), 5.37 (br s, 1H), 3.21-3.17 (m, 1H), 2.89 (dt, 1H, J=11.2, 7.3 Hz), 2.64-2.59 (m, 1H), 1.96 (dd, 1H, J=10.3, 6.3 Hz), 1.45-1.12 (m, 5H), 1.42 (s, 3H), 1.14 (s, 3H). IR (NaCl, thin film), cm⁻¹ 3222 (w), 2961 (w), 2927 (w), 1701 (s), 1684 (s). HRMS-ESI (m/z): [M+H]⁺ calcd for C₂₇H₂₆N₃O₃⁺, 440.1969; found, 440.1986.

### B. Biology

General Experimental Procedures. All cell-culture work was conducted in a class II biological safety cabinet. Buffers were filter-sterilized (0.2 μm) prior to use. Antiproliferative assays and other operations requiring the handling of nitrone species were carried out in the dark to prevent the occurrence of photochemical rearrangement reactions. Compounds 1-7 were typically stored in the dark as 5 mM stock solutions in DMSO, and were kept at −80° C. Compounds 8-11 were stored at −80° C. as dry solids (100-μg portions). Stock solutions (5 mM in DMSO) were prepared immediately prior to use.

Materials. LNCaP, T-47D, and HeLa-S3 cells were purchased from ATCC. COS-7 cells were kindly provided by Professor Alan Saghatelian. All cells were cultured in RPMI 1640 (Mediatech) containing 10% fetal bovine serum (Hyclone), 10 mM HEPES, and 2 mM L-glutamine. Cells were grown in BD Falcon tissue culture flasks with vented caps. Bradford reagent and Laemmli loading buffer (2× concentration) were purchased from Sigma Aldrich. Antiproliferative assays were conducted in pre-sterilized 96-well flat-bottomed plates from BD Falcon. Solutions of resazurin were purchased from Promega as part of the CellTiter-Blue Cell Viability Assay kit, and were used according to the manufacturer's instructions. Sodium dodecylsulfate polyacrylamide gel electrophoresis (SDS-PAGE) was performed using precast Novex tris-glycine mini gels (10%, 12% or 4-20% gradient, Invitrogen). Electrophoresis and semi-dry electroblotting equipment was purchased from Owl Separation Systems. Nitrocellulose membranes were purchased from Amersham Biosciences. A mouse monoclonal antibody to nucleophosmin (B23) was purchased from Santa Cruz Biotechnology (sc-32256). A rabbit polyclonal antibody to peroxiredoxin 1 was purchased from GeneTex (GTX15571). Rabbit polyclonal antibodies to exportin 1 and p53 were purchased from Santa Cruz Biotechnology (XPO1: sc-5595; p53: sc-6243). An Alexafluor 647 goat anti-mouse secondary antibody, together with Image-iT FX Signal Enhancer blocking solution, was purchased from Invitrogen (A31625). Western-blot detection was performed using the SuperSignal West Pico Chemiluminscence kit (including a goat anti-rabbit-HRP or goat anti-mouse-HRP conjugate) from Pierce. Western blots were visualized using CL-XPosure X-ray film from Pierce, or were imaged on an AlphaImager. Streptavidin-agarose was purchased from Sigma Aldrich. Protein bands were visualized using the Novex Colloidal Blue staining kit from Invitrogen, and were analyzed at the Taplin Biological Mass Spectrometry Facility (Harvard University). Yo-Pro iodide was purchased from Invitrogen.

Instrumentation. Absorbance and fluorescence measurements were made using Molecular Dynamics multiwell plate readers (absorbance: SPECTRAmax PLUS 384, fluorescence: SPECTRAmax GEMINI XS). Data was collected using SOFTmax PRO v. 4.3 (Molecular Dynamics), and was manipulated in Excel (Microsoft). The XLfit4 plugin (IDBS software) running in Excel was used for curve fitting. Analytical HPLC measurements were made on a Beckman Coulter System Gold HPLC, equipped with a reverse phase Beckman Coulter Ultrasphere ODS column (5 μM, 4.6 mm×25 cm). Fluorescence microscopy experiments were performed using a Zeiss upright microscope, equipped with 355 nm, 488 nm, 543 nm and 633 nm lasers. Flow cytometry experiments were performed on an LSR II flow cytometer (BD Biosciences).

### Preparation of Solutions.

RIPA buffer:
50 mM Tris•HCl, pH 7.35
150 mM NaCl
1 mM EDTA
1% Triton X-100
1% Sodium deoxycholate
0.1% SDS
1 mM PMSF
5 μg/mL aprotinin
5 μg/mL leupeptin
200 μM Na₃VO₄
50 mM NaF
Apoptosis Detection Buffer
100 nM Yo-Pro iodide
1.5 μM Propidium iodide
1 mM EDTA
1% BSA
in PBS
Wash buffer:
50 mM Tris•HCl, pH 7.6
75 mM NaCl
0.5 mM EDTA
0.5% Triton X-100
0.5% Sodium deoxycholate
0.05% SDS
Tris Buffer:
50 mM Tris•HCl, pH 7.8
Sucrose-Hypotonic Buffer:
25 mM Tris•HCl, pH 6.8
250 mM Sucrose
0.05% digitonin
1 mM DTT
1 mM PMSF
5 μg/mL leupeptin
200 μM Na₃VO₄
50 mM NaF

### Preparation of Resins.

A 400-μL aliquot of Sepharose 6B suspension (Sigma) was transferred to a 1.5-mL centrifuge tube. Wash buffer (1.0 mL, see above for formulation) was added, and the resulting slurry was mixed for 5 min at 4° C. The resin was centrifuged (12000×g, 2 min, 4° C.), and the supernatant was discarded. The resin was washed twice with 1.0 mL wash buffer (each wash: 5 min mixing at 4° C., followed by 2 min centrifugation at 12000×g, 4° C.), then was suspended in 800 μL wash buffer and mixed thoroughly prior to use.

A 400-μL aliquot of streptavidin-agarose suspension (Sigma) was transferred to a 1.5-mL centrifuge tube. Wash buffer (1.0 mL, see above for formulation) was added, and the resulting slurry was mixed for 5 min at 4° C. The resin was centrifuged (12000×g, 2 min, 4° C.), and the supernatant was discarded. The resin was washed twice with 1.0 mL wash buffer (each wash: 5 min mixing at 4° C., followed by 2 min centrifugation at 12000×g, 4° C.), then was suspended in 800 μL wash buffer and mixed thoroughly prior to use.

### Antiproliferative Assays.

LNCaP and T-47D cells were grown to approximately 80% confluence, then were trypsinized, collected, and pelleted by centrifugation (10 min at 183×g). The supernatant was discarded, and the cell pellet was resuspended in fresh medium to achieve a concentration of approximately 1.0 to 1.5×10⁶ cells/mL. A sample was diluted 10-fold in fresh medium, and the concentration of cells was determined using a hemacytometer.

The cell suspension was diluted to 1.0×10⁵ cells/mL. A multichannel pipette was used to charge the wells of a 96-well plate with 100 μL per well of the diluted cell suspension. The plates were incubated for 24 h at 37° C. under an atmosphere of 5% CO₂.

The following day, a 6.5-μL aliquot of nitrone solution, at 5 mM in DMSO, was diluted in 643.5 μL of medium to achieve a working concentration of 50 μM. Serial dilutions were employed to generate a range of different concentrations for analysis. Finally, 100-μL aliquots of the diluted nitrone solutions were added to the wells containing adhered cells, resulting in final assay concentrations of up to 25 μM.

The treated cells were incubated for 72 h at 37° C. (5% CO₂). To each well was added 20 μL of CellTiter-Blue reagent, and the samples were returned to the incubator. Fluorescence (560 nm excitation/590 nm emission) was recorded on a 96-well plate reader following a 4.0 h incubation period (37° C., 5% CO₂).

Percent growth inhibition was calculated for each well, based upon the following formula:

Percent growth inhibition=100×(S−B₀)/(B_{t-B}₀)

where S is the sample reading, Bₜ is the average reading for a vehicle-treated population of cells at the completion of the assay, and B₀ is the average reading for an untreated population of cells at the beginning of the assay.

Each analogue was run a minimum of eight times, over a period of at least two weeks. For each compound, 14 separate concentrations were used in the assay, ranging from 25 μM to 8 nM. The average inhibition at each concentration was plotted against concentration, and a curve fit was generated. To eliminate positional effects (e.g., cell samples in the center of the plate routinely grew more slowly than those near the edge), the data was automatically scaled to ensure that the curves showed no inhibition at negligible concentrations of added compound. Such a precaution was found to generate more consistent data from week to week, without affecting the final results. Final GI₅₀ values reflect the concentrations at which the resulting curves pass through 50 percent inhibition.

### Fluorescence Microscopy Experiments.

HeLa-S3 cells were grown to approximately 80% confluence, then were trypsinized, collected, and pelleted by centrifugation (10 min at 183×g). The supernatant was discarded and the cell pellet was resuspended in fresh medium to achieve a concentration of approximately 1.0 to 1.5×10⁶ cells/mL. A sample was diluted 10-fold in fresh medium, and the concentration of cells was determined using a hemacytometer.

The cell suspension was diluted to 2.0×10⁴ cells/mL. A 6-well plate was charged with one 22 mm×22 mm number 1.5 glass coverslip per well, followed by 4 mL/well of cell suspension. The plate was incubated for 24 h at 37° C. under an atmosphere of 5% CO₂.

The following day, 5.94 μL of a 5 mM stock solution of probe 4 in DMSO was added to 1094 μL of cell-culture medium. From the resulting 27 μM solution, 500 μL was added to one well of the 6-well plate, resulting in a final concentration of 3 μM probe 4. Other samples were prepared in a similar manner, but with final concentrations of 1 μM or 0 μM (vehicle control) probe 4. All samples contained 0.06% DMSO.

The plate was returned to the incubator for 2 h, then the coverslips were carefully removed. Each coverslip was immersed in 5 mL methanol at −20° C. for 3 min to fix the cells, then was washed three times (5 min per wash) in 5 mL PBS. The cells were permeablized by immersing the coverslips in 5 mL of 0.1% Triton X-100 in PBS for 5 min at 23° C., followed by three washes (5 min in 5 mL PBS). The coverslips were coated with a film of Image-iT FX Signal Enhancer and incubated at 23° C. for 30 min, then were washed three times (5 min in 5 mL PBS).

The 3 μM and vehicle control samples were rinsed briefly in water, then mounted on slides with 20 μL Mowiol mounting mixture (containing 0.1% p-phenylene diamine).

The 1 μM sample was treated with 150 μL of primary antibody solution (0.5 μL of mouse anti-B23, Santa Cruz Biotechnology (sc-32256) in 499.5 μL PBS) for 30 min, then washed three times (5 min in 5 mL PBS) and treated with 150 μL of secondary antibody solution (0.5 μL of Alexafluor 647 goat anti-mouse, Invitrogen (A31625) in 499.5 μL PBS) for 30 min. The coverslip was washed three more times (5 min in 5 mL PBS), rinsed briefly in water, and mounted onto a slide with 20 μL Mowiol mounting mixture (containing 0.1% p-phenylene diamine).

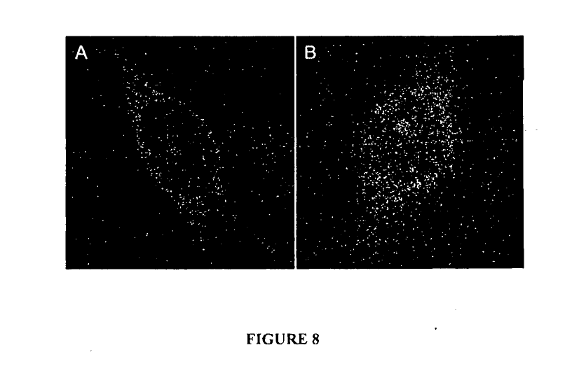

Fluorescence microscopy experiments (λₑₓ=355 nm) showed that the dansyl group of the activity-based probe 4 was detectable above the background; e.g., cells treated with 3 μM of probe 4 (FIG. 8A) showed a higher fluorescence output than cells treated with vehicle control (FIG. 8B).

Probe 4 was observed in both the cytosol and nucleus of HeLa S3 cells at concentrations of both 1 μM and 3 μM. Within the nucleus, the probe appeared to be concentrated within a smaller intranuclear region, identified as the nucleolus by immunofluorescence experiments using nucleophosmin as a nucleolar marker (FIG. 8B, FIG. 2).

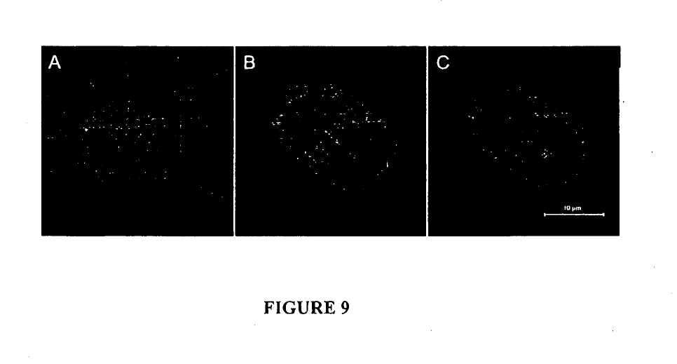

Data from similar experiments in T-47D cells are shown in FIG. 9.

Affinity-Isolation Experiments from Incubations with Live Cells, then Lysis
1. Preparation of Cellular Lysates from Treated Cells.

T-47D cells were grown to approximately 80% confluence, then were trypsinized, collected, and pelleted by centrifugation (10 min at 183×g). The supernatant was discarded, and the cell pellet was resuspended in fresh medium to achieve a concentration of approximately 1.0 to 1.5×10⁶ cells/mL. A sample was diluted 10-fold in fresh medium, and the concentration of cells was determined using a hemacytometer.

The cell suspension was diluted to 3.0×10⁵ cells/mL. Cell culture flasks (75 cm²) were charged with 12 mL of the suspension, and were then incubated for 2 d at 37° C. under an atmosphere of 5% CO₂. The medium was removed, and 12 mL fresh cell culture medium was added. Incubation was continued for 24 h. The cells were ˜65% confluent.

The medium was removed from the growing cells, and replaced with 12 mL of medium containing the following activity-based probes and control compounds (from 5 mM stocks in DMSO):

| | volume | volume | volume 5 | | volume 3 | volume (+)-1 | volume 2 | volume 7 | % |
|---|---|---|---|---|---|---|---|---|---|
| sample: | medium | DMSO | 5000 μM | | 5000 μM | 5000 μM | 5000 μM | 5000 μM | DMSO |
| 1 | 12.5 mL | 45.0 μL | x | | x | x | x | x | 0.36% |
| 2 | 12.5 mL | 37.5 μL | 7.5 μL | (3 μM) | x | x | x | x | 0.36% |

| | | | | | | | | | | |
|---|---|---|---|---|---|---|---|---|---|---|
| 3 | 12.5 mL | 30.0 μL | x | x | 7.5 μL | (3 μM) | x | 7.5 μL | (3 μM) | 0.36% |

| | | | | | | | | | |
|---|---|---|---|---|---|---|---|---|---|
| 4 | 12.5 mL | 22.5 μL | x | 22.5 μL | (9 μM) | x | x | x | 0.36% |

| | | | | | | | | | | |
|---|---|---|---|---|---|---|---|---|---|---|
| 5 | 12.5 mL | x | x | x | x | 22.5 μL | (9 μM) | 22.5 μL | (9 μM) | 0.36% |

The cells were incubated for 90 min at 37° C. under an atmosphere of 5% CO₂. The medium (including any detached cells) from each sample was transferred to a 50-mL centrifuge tube. The cells were rinsed with 10 mL PBS, which was added to the centrifuge tubes. Adhered cells were detached from the culture flask by trypsinization (10 min, 37° C., 5 mL per flask, 0.05% trypsin, 0.53 mM EDTA). Fresh medium (10 mL) was added and the resulting suspension was added to the centrifuge tubes, along with a 5-mL PBS rinse.

The samples were centrifuged (10 min at 183×g), and the supernatant was discarded. The cells were resuspended in 1 mL of PBS, the suspension was transferred to a 1.5-mL centrifuge tube, and the cells were again pelleted by centrifugation (5 min at 500×g). The supernatant was discarded, and the cells were washed twice with 1 mL of PBS.

The washed cells were cooled on ice, then were lysed by addition of 500 μL per sample ice-cold RIPA buffer (see above for formulation). The samples were mixed end-over-end for 1 hour at 4° C. with occasional vortexing, then 500 μL per sample Tris buffer was added. The samples were centrifuged (12000×g, 10 min, 4° C.), and insoluble material was removed with a pipette tip. The lysates were transferred to fresh 1.5-mL centrifuge tubes.

### 2. Affinity-Isolation of Bound Proteins.

Each individual sample lysate from section 1 was treated with 50 μL of washed, well-suspended, two-fold diluted Sepharose resin (see above for resin preparation). The resulting slurry was mixed for 6 h at 4° C., then was centrifuged (12000×g, 2 min, 4° C.). The supernatant was transferred to a clean 1.5 mL centrifuge tube. The protein concentration in each lysate was analyzed by the Bradford method, and found to be consistent across all samples, within experimental error.

Each sample was treated with two 30-μL aliquots of washed, well-suspended, two-fold diluted streptavidin-agarose resin (see above for resin preparation). The resulting slurry was mixed for 15 h at 4° C., then was centrifuged (12000×g, 10 min, 4° C.). The supernatant was discarded.

The collected resins were washed with wash buffer at 4° C., then with tris buffer at 4° C., then twice with tris buffer at 23° C. Each wash consisted of 10 min mixing, followed by 10 min centrifugation (either 12000×g at 4° C., or 10000×g at 23° C.). See above for solution preparation.

The washed resin was suspended in Laemmli loading buffer (Sigma, 2× concentration, 70 μL per sample), and the samples were heated to 95° C. for 6 min.

### 3. Western-Blot Detection of Nucleophosmin.

A tris-glycine mini gel (4-20%, 12-well) was loaded with 15 μL per lane of the denatured protein mixture from section 2. One lane was loaded with 8 μL of Benchmark prestained protein ladder (Invitrogen). The protein samples were electroeluted (1 h, 23° C., 150 V), then transferred under semi-dry conditions to a nitrocellulose membrane (100 mA, 23° C., 12 h).

The membrane was blocked for 1 h (40 mL 3% low-fat milk in TBS buffer with 0.1% tween-20), then rinsed (two ten min washes with TBS buffer containing 0.1% tween-20), and treated 1 h with primary antibody solution (20 mL of 1% low-fat milk in TBS buffer with 0.1% tween-20, containing 10 μg of mouse anti-B23 antibody). The membrane was rinsed again (two 10-min washes with 40 mL TBS buffer containing 0.1% tween-20) and treated with secondary antibody solution (20 mL of 1% low-fat milk in TBS buffer with 0.1% tween-20, containing 20 μg of goat anti-mouse-HRP conjugate). The membrane was rinsed once more (three 10-min washes with 40 mL TBS buffer containing 0.1% tween-20) and treated with 6 mL of a 1:1 mixture of stabilized peroxide solution:enhanced luminol solution (Pierce; WestPico Chemiluminescent Substrate kit) for 3 min. Finally, the membrane was sealed in plastic wrap and exposed to X-ray film to provide the Western-blot of FIG. 3A.

Affinity-Isolation Experiments from Incubations with Cell Lysates

### 1. Preparation of Whole Cell Lysate

T-47D cells were grown to approximately 90% confluence in 9 T-150 tissue culture flasks. The medium was discarded, and the cells were washed with PBS (10 mL per flask). The cells were harvested by trypsinization (10 min, 37° C., 8 mL per flask, 0.05% trypsin, 0.53 mM EDTA). Fresh cell-culture medium (16 mL) was added to each flask, and the suspension was transferred to 50-mL centrifuge tubes. The cells were pelleted by centrifugation (10 min at 183×g). The supernatant was discarded, and the cell pellets were resuspended in PBS (10 mL) and transferred to 15-mL centrifuge tubes. The cells were pelleted once again by centrifugation (10 min at 183×g), then were washed twice with 5 mL PBS.

Packed cells (1.5 mL) were cooled on ice. Ice-cold RIPA buffer (5 mL, see above for formulation) was added, and the mixture was rotated end-over-end for 1 h at 4° C. Tris buffer (5 mL) was added, and the lysate was centrifuged (12000×g, 10 min, 4° C.). Insoluble material was removed with a pipette tip, and the remaining lysate was transferred to a clean 15-mL centrifuge tube. A 750-μL aliquot of washed, well-suspended, two-fold diluted streptavidin-agarose resin (see above for resin preparation) was added, and the resulting slurry was mixed for 5 h at 4° C., then was centrifuged (12000×g, 10 min, 4° C.). The supernatant lysate was carefully removed, briefly mixed, and partitioned into ten 1-mL aliquots, which were flash-frozen in liquid N₂ and stored at −80° C. prior to use. The lysate contained 7.6 mg/mL total protein (Bradford method).

### 2. Preparation of Nuclear-Enriched Lysate.

T-47D cells were grown to approximately 90% confluence in 11 T-150 tissue culture flasks. The medium was discarded, and the cells were washed with PBS (10 mL per flask), then harvested by trypsinization (10 min, 37° C., 8 mL per flask, 0.05% trypsin, 0.53 mM EDTA). Fresh cell-culture medium (16 mL) was added to each flask, and the resulting suspension was transferred to 50-mL centrifuge tubes. The cells were pelleted by centrifugation (10 min at 183×g). The supernatant was discarded, and the cell pellets were resuspended in PBS (10 mL) and transferred to a 15-mL centrifuge tubes. The cells were pelleted once again by centrifugation (10 min at 183×g), then were washed twice with 5 mL PBS.

Packed cells (2.1 mL) were cooled on ice. Ice-cold sucrose-hypotonic buffer (5 mL, see above for formulation) was added. The suspension was mixed for 1 min on ice, then was centrifuged (6800×g, 3 min, 4° C.). The supernatant (cytosolic lysate) was removed, and the remaining pellet was washed twice with 4 mL PBS, then was lysed by the addition of 6 mL RIPA buffer (see above for formulation). The suspension was mixed end-over-end for 1 h at 4° C., then was diluted with 6 mL tris buffer and centrifuged (12000×g, 10 min, 4° C.). Insoluble material was removed using a pipette tip, and the remaining nuclear-enriched lysate was carefully removed, briefly mixed, and partitioned into ten 1-mL aliquots, which were flash-frozen in liquid N₂ and stored at −80° C. prior to use. The lysate contained 6.2 mg/mL total protein (Bradford method).

### 3. Titration of Probe 5-Nucleophosmin Binding.

A 1-mL aliquot of T-47D whole cell lysate was thawed at 4° C. and diluted with 4 mL wash buffer, to afford a working lysate of 1.5 mg/mL total protein. This was partitioned into 1.5-mL centrifuge tubes, and treated (on ice, in the dark) with DMSO and solutions of 5 (prepared by serial dilution from an initial 5 mM stock in DMSO) as indicated:

| | volume | volume | volume 5 | | volume 5 | volume 5 | final | % |
|---|---|---|---|---|---|---|---|---|
| sample: | lysate | DMSO | 5 μM | | 50 μM | 500 μM | volume | DMSO |
| 1 | 384 μL | 16 μL | x | | x | x | 400 μL | 4% |
| 2 | 384 μL | 8 μL | 8 μL | (100 nM) | x | x | 400 μL | 4% |

| | | | | | | | | |
|---|---|---|---|---|---|---|---|---|
| 3 | 384 μL | 12 μL | x | 4 μL | (500 nM) | x | 400 μL | 4% |
| 4 | 384 μL | 8 μL | x | 8 μL | (1 μM) | x | 400 μL | 4% |

| | | | | | | | | |
|---|---|---|---|---|---|---|---|---|
| 5 | 384 μL | 8 μL | x | x | 8 μL | (10 μM) | 400 μL | 4% |

The samples were mixed end-over-end in the dark for 4 h at 4° C. Each sample was treated with two 30-μL aliquots of washed, well-suspended, two-fold diluted streptavidin-agarose resin (see above for resin preparation). The resulting slurry was mixed for 15 h at 4° C., then was centrifuged (12000×g, 10 min, 4° C.). The supernatant was discarded.

The collected resins were washed with wash buffer at 4° C., then with tris buffer at 4° C., then twice with tris buffer at 23° C. Each wash consisted of 10 min mixing, followed by 10 min centrifugation (either 12000×g at 4° C., or 10000×g at 23° C.). See above for solution preparation.

The washed resin was suspended in Laemmli loading buffer (Sigma, 2× concentration, 90 μL per sample), and the samples were heated to 95° C. for 6 min.

A tris-glycine mini gel (4-20%, 12-well) was loaded with 15 μL per lane of the denatured protein mixture. One lane was loaded with 8 μL of Benchmark prestained protein ladder (Invitrogen). The protein samples were electroeluted (1 h, 23° C., 150 V), then transferred under semi-dry conditions to a nitrocellulose membrane (100 mA, 23° C., 12 h).

The membrane was blocked for 1 hour (40 mL 3% low-fat milk in TBS buffer with 0.1% tween-20), then rinsed (two 10-min washes with TBS buffer containing 0.1% tween-20), and treated 1 h with primary antibody solution (20 mL of 1% low-fat milk in TBS buffer with 0.1% tween-20, containing 10 μg of mouse anti-B23 antibody). The membrane was rinsed again (two 10-min washes with 40 mL TBS buffer containing 0.1% tween-20) and treated with secondary antibody solution (20 mL of 1% low-fat milk in TBS buffer with 0.1% tween-20, containing 20 μg of goat anti-mouse-HRP conjugate). The membrane was rinsed once more (three 10-min washes with 40 mL TBS buffer containing 0.1% tween-20) and treated with 6 mL of a 1:1 mixture of stabilized peroxide solution:enhanced luminol solution (Pierce; WestPico Chemiluminescent Substrate kit) for 3 min. Finally, the membrane was sealed in plastic wrap and exposed to X-ray film to provide the Western-blot of FIG. 3B.

### 4. Competitive Binding Affinity-Isolation Experiments.

Aliquots of T-47D whole cell and nuclear-enriched lysates were thawed at 4° C. and diluted with wash buffer to provide working lysates of 1.5 mg/mL total protein. These were partitioned into 1.5-mL centrifuge tubes, and treated (on ice, in the dark) with DMSO and solutions of 5, 1, ent-1 and 2, as indicated:

| | volume | volume | volume 5 | | volume 1 | | volume ent-1 | volume 2 | final | % |
|---|---|---|---|---|---|---|---|---|---|---|
| sample: | lysate | DMSO | 500 μM | | 5 mM | | 5 mM | 5 mM | volume | DMSO |
| 1 A | nuclear | 8 μL | 8 μL | (10 μM) | x | | x | x | 400 μL | 4% |
| | 384 μL | | | | | | | | | |
| 2 A | nuclear | 0 μL | 8 μL | (10 μM) | 8 μL | (100 μM) | x | x | 400 μL | 4% |
| | 384 μL | | | | | | | | | |

| | | | | | | | | | | |
|---|---|---|---|---|---|---|---|---|---|---|
| 3 A | nuclear | 0 μL | 8 μL | (10 μM) | x | 8 μL | (100 μM) | x | 400 μL | 4% |
| | 384 μL | | | | | | | | | |

| | | | | | | | | | | |
|---|---|---|---|---|---|---|---|---|---|---|
| 4 A | nuclear | 0 μL | 8 μL | (10 μM) | x | x | 8 μL | (100 μM) | 400 μL | 4% |
| | 384 μL | | | | | | | | | |

| 1 B | whole cell | 8 μL | 8 μL | (10 μM) | x | | x | x | 400 μL | 4% |
|---|---|---|---|---|---|---|---|---|---|---|
| | 384 μL | | | | | | | | | |
| 2 B | whole cell | 0 μL | 8 μL | (10 μM) | 8 μL | (100 μM) | x | x | 400 μL | 4% |
| | 384 μL | | | | | | | | | |

| | | | | | | | | | | |
|---|---|---|---|---|---|---|---|---|---|---|
| 3 B | whole cell | 0 μL | 8 μL | (10 μM) | x | 8 μL | (100 μM) | x | 400 μL | 4% |
| | 384 μL | | | | | | | | | |

| | | | | | | | | | | |
|---|---|---|---|---|---|---|---|---|---|---|
| 4 B | whole cell | 0 μL | 8 μL | (10 μM) | x | x | 8 μL | (100 μM) | 400 μL | 4% |
| | 384 μL | | | | | | | | | |

The samples were mixed end-over-end in the dark for 4 h at 4° C. Each sample was treated with two 30-μL aliquots of washed, well-suspended, two-fold diluted streptavidin-agarose resin (see above). The resulting slurry was mixed for 15 h at 4° C., then was centrifuged (12000×g, 10 min, 4° C.). The supernatant was discarded.

The collected resins were washed with wash buffer at 4° C., then with tris buffer at 4° C., then twice with tris buffer at 23° C. Each wash consisted of 10 min mixing, followed by 10 min centrifugation (either 12000×g at 4° C., or 10000×g at 23° C.). See above for solution preparation.

The washed resin was suspended in Laemmli loading buffer (Sigma, 2× concentration, 90 μL per sample), and the samples were heated to 95° C. for 6 min.

A tris-glycine mini gel (4-20%, 12-well) was loaded with 15 μL per lane of the denatured protein mixture. One lane was loaded with 8 μL of Benchmark prestained protein ladder (Invitrogen). The protein samples were electroeluted (1 h, 23° C., 150 V), then transferred under semi-dry conditions to a nitrocellulose membrane (100 mA, 23° C., 12 h).

The membrane was blocked for 1 h (40 mL 3% low-fat milk in TBS buffer with 0.1% tween-20), then rinsed (two 10-min washes with TBS buffer containing 0.1% tween-20), and treated 1 h with primary antibody solution (20 mL of 1% low-fat milk in TBS buffer with 0.1% tween-20, containing 10 μg of mouse anti-B23 antibody). The membrane was rinsed again (two 10-min washes with 40 mL TBS buffer containing 0.1% tween-20) and treated with secondary antibody solution (20 mL of 1% low-fat milk in TBS buffer with 0.1% tween-20, containing 20 μg of goat anti-mouse-HRP conjugate). The membrane was rinsed once more (three 10-min washes with 40 mL TBS buffer containing 0.1% tween-20) and treated with 6 mL of a 1:1 mixture of stabilized peroxide solution:enhanced luminol solution (Pierce; WestPico Chemiluminescent Substrate kit) for 3 min. Finally, the membrane was sealed in plastic wrap and exposed to X-ray film to provide the Western-blot of FIG. 3C.

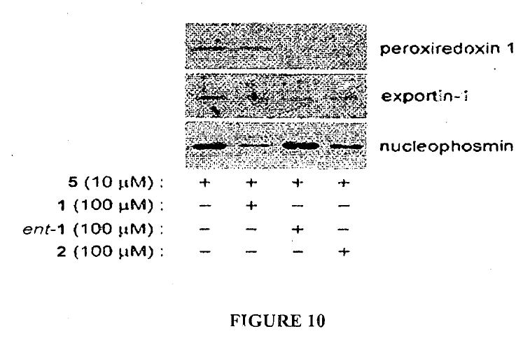

Western-blot detection of exportin-1 (XPO1) and peroxiredoxin 1 (PRX1) showed that all three inhibitors (1, ent-1 and 2) were capable of blocking the binding of probe 5 to these proteins whereas the three inhibitors exhibited differential blocking of the binding of probe 5 to nucleophosmin, with the natural product 1 being most effective (FIG. 10).

5. Affinity-Isolation Experiments following Co-Incubation with Iodoacetamide.

Identical affinity-isolation experiments to those described in the previous section were performed, except that iodoacetamide (8 μL of a freshly prepared 500 mM solution in DMSO) was added to one sample:

| | | | volume 5 | | volume | | | |
|---|---|---|---|---|---|---|---|---|
| | volume | volume | 500 | | iodoacetamide | | final | % |
| sample: | lysate | DMSO | μM | | 500 mM | | volume | DMSO |
| 1 | whole cell | 8 μL | 8 μL | (10 μM) | x | | 400 μL | 4% |
| | 384 μL | | | | | | | |
| 2 | whole cell | 0 μL | 8 μL | (10 μM) | 8 μL | (10 mM) | 400 μL | 4% |
| | 384 μL | | | | | | | |

Western-blot detection (as described above) revealed a reduction in affinity-isolated nucleophosmin for the sample treated with iodoacetamide.

### Example 2

### Avrainvillamide Shows Selectivity for Malignant versus Non-Malignant Cells

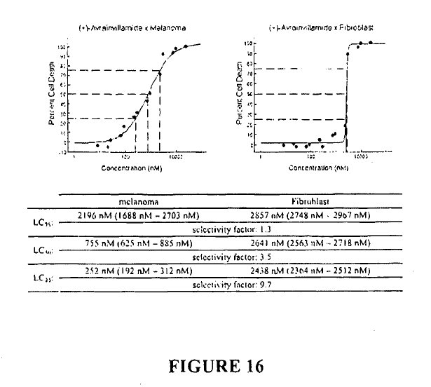

Avrainvillamide shows nanomolar activity against MALME-3M cells, which corresponds to a malignant metastatic melanoma isolated from the lung of a 43 y.o. Caucasion male. A cell line from a healthy fibroblast from the same patient has also been deposited with the American Type Cell Culture Corporation (ATCC). Fresh stockes of both MALME-3M and MALME-3 from ATCC. Avrainvillamide was test against the two cells lines at the same time, taking all possible precautions to ensure that both sets of samples were treated identically. FIG. 16 shows the data from this study. As a measure of cytotoxicity and antiproliferative activity, we calculated both LC50 and LC25 (as an estimate of GI50). Avrainvillamide showed a significantly greater activity against the melanoma cells relative to the fibroblast control, with selectivity factors of 3.5 and 9.7 for the two different measurements.

The 9.7-fold selectivity at 25 percent cell death is representative of at least a modest degree of selectivity. For comparison, cytochalsine B and geldanamycin were analyzed in identical experiments. Cytochalasine B is a non-selective cytotoxic agent for which a selectivity factor of 0.2 at 25 percent cell death was observed. Geldanamycin is known to be a potent, selective inhibitor of tumor cell growth for which a selectivity factor of >100 at 25 percent cell death was observed. In sum, these results indicate that avrainvillamide has a modest degree of selectivity for malignant cells.

Morphologically, the avrainvillamide's effect against the two cell lines was even more striking. When treated with avrainvillamide, cells display partial detachment along with balling up of the cell structure. Cytochalasin B induced this morphological change in both melanoma and fibroblast cells. In contrast, avrainvillamide did not cause this type of change in fibroblast cells, which may suggest a different mechanism of action. If the cytotoxicity of avrainvillamide in fibroblast cells is in fact due to off-target drug-protein interactions, then it may be possible to design an analogue with even greater selectivity.

### Example 3

### In Vitro Cytotoxicity Data for Avrainvillamide Analogs

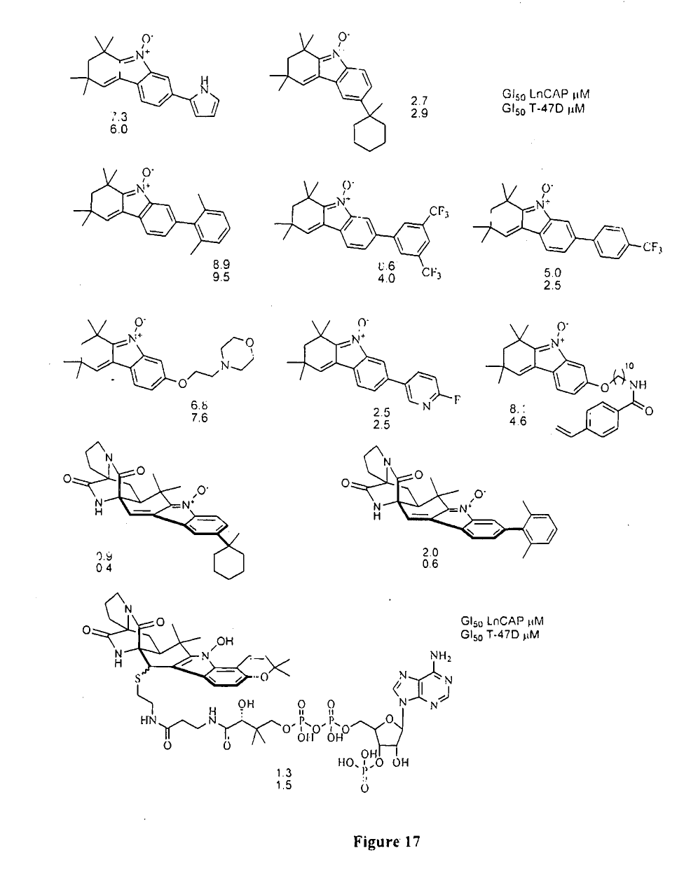

In vitro cytotoxicity data for several analogs of avrainvillamide in LnCAP and T-47D cells are shown in FIG. 17. LnCap cells are human androgen-sensitive human prostate adenocarcinoma cells, and T-47D are human breast ductal carcinoma cells.

In addition, five potent analogues of avrainvillamide as shown below were tested in the NCI 60 cell lines.

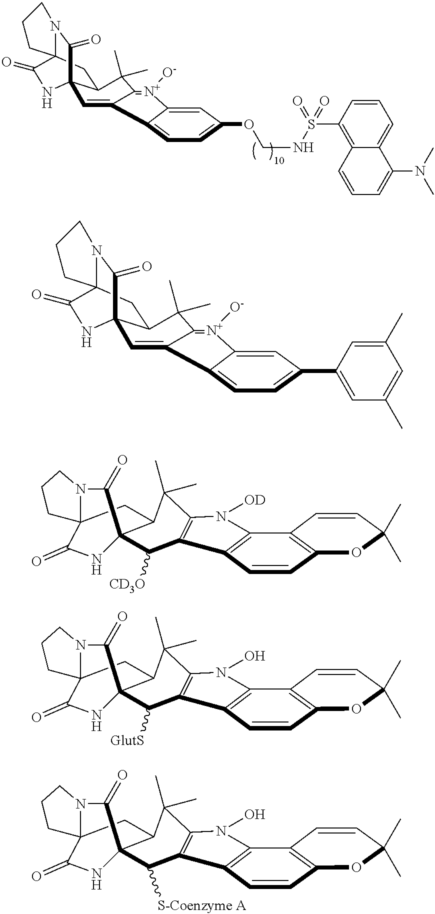

The human tumor cell lines were grown in RPMI 1640 medium containing 5% fetal bovine serum (FBS) and 2 mM L-glutamine. The cells were inoculated into 96 well microtiter plates in 100 μL volumes at plating densities ranging from 5000 to 40000 cells/well depending on the doubling time of each individual cell line. After cell inoculation, the microtiter plates were incubated at 37° C., 5% CO₂, 95% air, and 100% relative humidity for 24 hours prior to addition of the test compound. The following day, two plates of each cell line were fixed in situ with TCA to represent a measurement of cell population for each cell line at the time of sample addition (T_{z}). Each of the test compounds was dissolved in dimethyl sulfoxide at 400-times the desired final maximum test concentration, and the resulting solutions were stored frozen prior to use. At the time of sample addition, an aliquot of frozen concentrate was thawed and diluted to twice the desired final maximum test concentration with complete medium containing 50 μg/mL gentamicin. Additional four 10-fold serial dilutions were prepared to provide a total of five sample concentrations plus control. Aliquots of 100 μL of these different concentrations were added to the appropriate microtiter wells already containing 100 μL of medium, making up the required final sample concentrations. After addition of the test compound to the cell lines, the plates were incubated for an additional 48 hours at 37° C., 5% CO₂, 95% air, and 100% relative humidity. For adherent cells, the assay was terminated by the addition of cold TCA. Cells were fixed in situ by gentle addition of 50 μL of cold 50% (w/v) TCA (final concentration of 10% TCA), and the plates were incubated for 60 minutes at 4° C. The supernatant was discarded, and the plates were washed five times with tap water and air-dried. Sulforhodamine B (SRB) solution (100 μL at 0.4% w/v in 1% acetic acid) was added to each well, followed by incubation for 10 minutes at room temperature. After staining, unbound dye was removed by washing five times with 1% acetic acid, and the plates were air-dried. Bound stain was subsequently solubilized with 10 mM trizma base, and the absorbance was read on an automated plate reader at a wavelength of 515 nm. For suspension cells, the same methodology was applied except that the assay was terminated by fixing settled cells at the bottom of the wells by gentle addition of 50 μL of 80% TCA (final concentration=16% TCA). Using the seven absorbance measurements [time zero (T_{z}), control growth (C), and test growth in the presence of drug at the five concentration levels (Tᵢ)], the percentage growth was calculated at each of the sample concentration levels. Percentage growth inhibition=[(Tᵢ−T_{z})/(C−T_{z})]×100 for concentrations for which Tᵢ≧T_{z}; or Percentage growth inhibition=[(Tᵢ−T_{z})/T_{z}]×100 for concentrations for which Tᵢ<T_{z}. Three dose response parameters were computed for each experimental cell line. Growth inhibition of 50% (GI₅₀) was calculated from [(Tᵢ−T_{z})/(C−T_{z})]×100=50, representing the sample concentration resulting in a 50% reduction in the net protein increase (as measured by SRB staining) in control cells during incubation. The test compound concentration resulting in total growth inhibition was calculated from Tᵢ=T_{z}. The lethal concentration (LC₅₀, concentration of drug resulting in a 50% reduction in the measured protein at the end of the treatment as compared to that at the beginning) was calculated from [(Tᵢ−T_{z})/T_{z}]×100=−50. In the event when the effect was not reached or was exceeded, the value for the respective parameter was expressed as greater or less than the maximum or minimum concentration tested.

The NCI 60 cells lines used are listed in the table below.

| Panel | Cell Line | Panel | Cell Line |
|---|---|---|---|
| Leukemia | CCRF-CEM | Colon Cancer | COLO 205 |
| | HL-60(TB) | | HCC-2998 |
| | K-562 | | HCT-116 |
| | MOLT-4 | | HCT-15 |
| | RPMI-8226 | | HT29 |
| | SR | | KM12 |
| Non-Small Cell | A549/ATCC | | SW-620 |
| Lung Cancer | EKVX | CNS Cancer | SF-268 |
| | HOP-62 | | SF-295 |
| | HOP-92 | | SF-539 |
| | NCI-H226 | | SNB-19 |
| | NCI-H23 | | SNB-75 |
| | NCI-H322M | | U251 |
| | NCI-H460 | Melanoma | LOX IMVI |
| | NCI-H522 | | MALME-3M |
| Colon Cancer | COLO 205 | | M14 |
| | HCC-2998 | | SK-MEL-2 |
| | HCT-116 | | SK-MEL-28 |
| | HCT-15 | | SK-MEL-5 |
| | HT29 | | UACC-257 |
| | KM12 | | UACC62 |
| | SW-620 | Renal Cancer | 786-0 |
| Ovarian Cancer | IGROV1 | | A498 |
| | OVCAR-3 | | ACHN |
| | OVCAR-4 | | CAKI-1 |
| | OVCAR-5 | | RXF 393 |
| | OVCAR-8 | | SN12C |
| | SK-OV-3 | | TK-10 |
| Prostate Cancer | PC-3 | Prostate Cancer | DU-145 |
| Breast Cancer | MCF7 | Breast Cancer | MDA-MB-435 |
| | NCI/ADR-RES | | BT-549 |
| | MDA-MB-231/ATCC | | T-47D |
| | HS578T | | MDA-MB-468 |

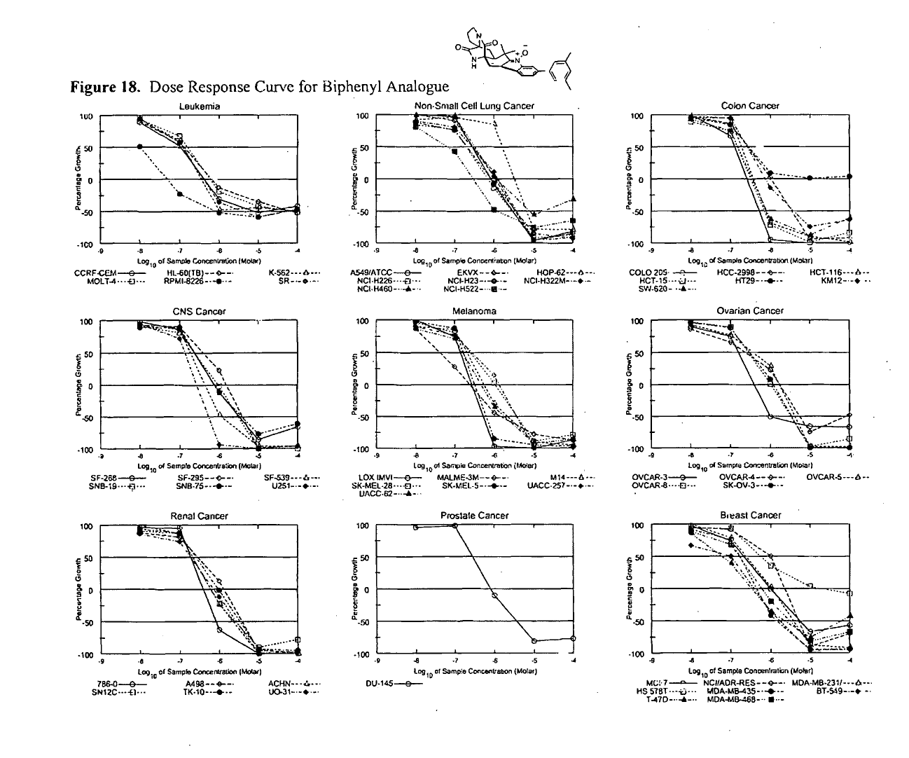

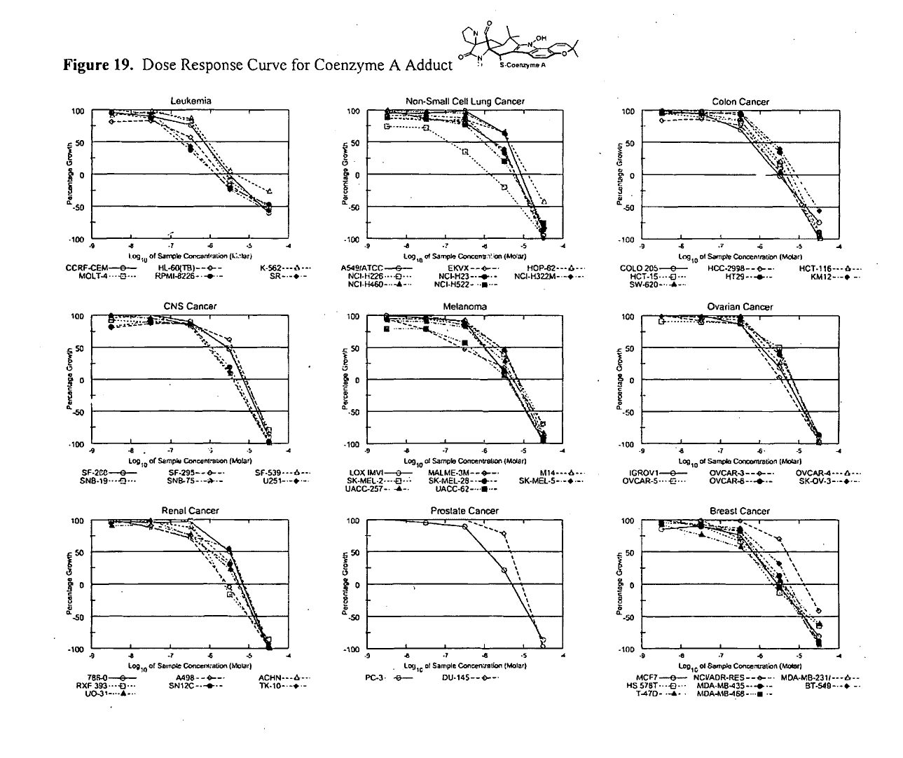

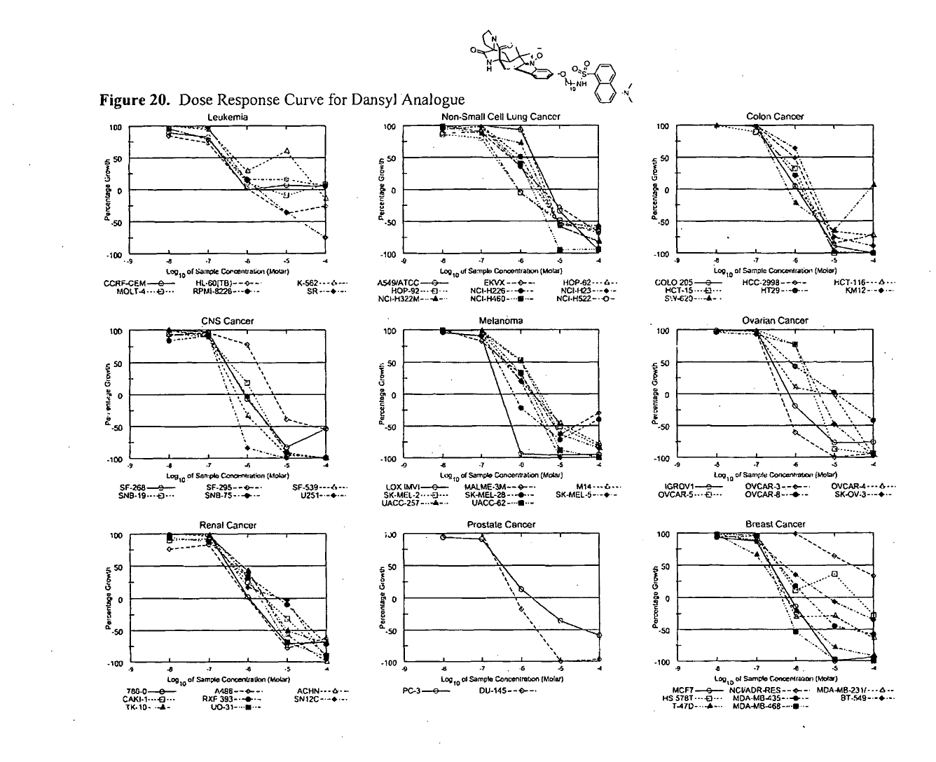

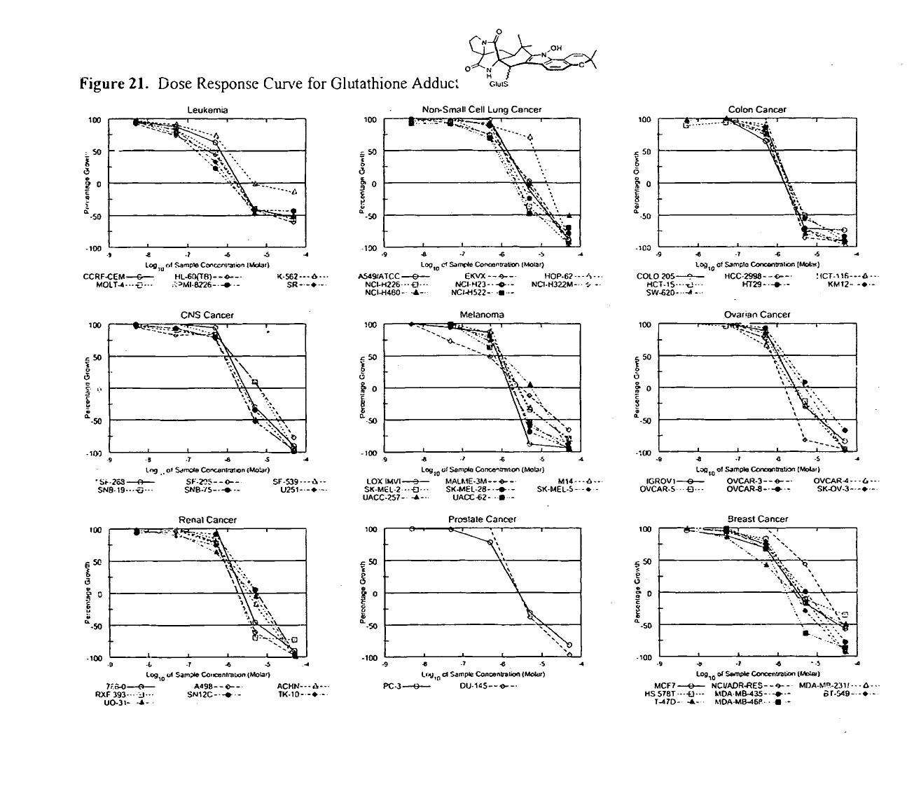

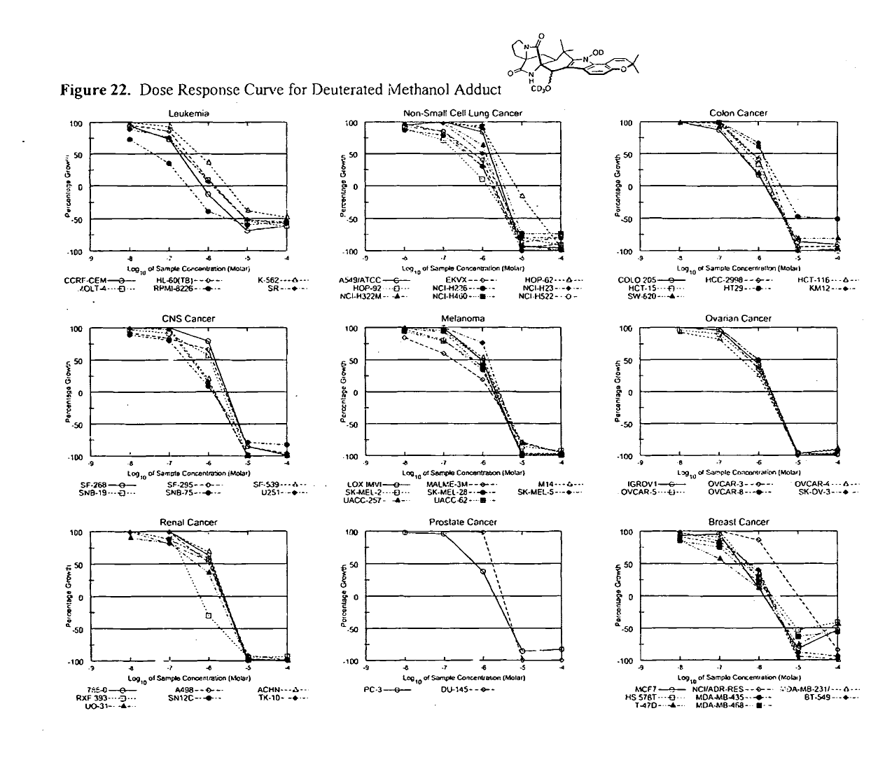

These results include GI₅₀, TGI, and LC₅₀ values for each compound in the 60 cell lines as shown in the tables below. These analogues showed sub-micromolar inhibition towards most of these cells lines. Representative dose-response curves for the five analogues are included as FIGS. 18-22.

Dansyl Analogue

| Panel | Cell Line | GI50 (M) | TGI (M) | LC50 (M) |
|---|---|---|---|---|
| Leukemia | CCRF-CEM | 2.46E−07 | >1.00E−4 | >1.00E−4 |
| Leukemia | HL-60(TB) | 2.11E−07 | 1.16E−06 | >1.00E−4 |
| Leukemia | K-562 | — | 6.44E−05 | — |
| Leukemia | MOLT-4 | 2.76E−07 | — | >1.00E−4 |
| Leukemia | RPMI-8226 | 2.82E−07 | >1.00E−4 | >1.00E−4 |
| Leukemia | SR | 3.71E−07 | 1.94E−06 | 2.37E−05 |
| Non-Small Cell | A549/ATCC | 2.17E−06 | 5.37E−06 | 1.81E−05 |
| Lung Cancer | | | | |
| Non-Small Cell | EKVX | 5.40E−07 | 3.63E−06 | 3.70E−05 |
| Lung Cancer | | | | |
| Non-Small Cell | HOP-62 | 2.38E−06 | 4.64E−06 | 9.02E−06 |
| Lung Cancer | | | | |
| Non-Small Cell | HOP-92 | 2.24E−07 | 8.67E−07 | 1.27E−05 |
| Lung Cancer | | | | |
| Non-Small Cell | NCI-H226 | 1.03E−06 | 3.03E−06 | 8.93E−06 |
| Lung Cancer | | | | |
| Non-Small Cell | NCI-H23 | 5.81E−07 | 2.48E−06 | 9.05E−06 |
| Lung Cancer | | | | |
| Non-Small Cell | NCI-H322M | 1.48E−06 | 3.58E−06 | 8.67E−06 |
| Lung Cancer | | | | |
| Non-Small Cell | NCI-H460 | 7.00E−07 | 1.99E−06 | 4.64E−06 |
| Lung Cancer | | | | |
| Non-Small Cell | NCI-H522 | 2.60E−07 | 8.57E−07 | 8.89E−06 |
| Lung Cancer | | | | |
| Colon Cancer | COLO 205 | 3.28E−07 | 1.14E−06 | 3.86E−06 |
| Colon Cancer | HCC-2998 | 9.46E−07 | 2.32E−06 | 5.49E−06 |
| Colon Cancer | HCT-116 | 3.49E−07 | 1.15E−06 | 5.98E−06 |
| Colon Cancer | HCT-15 | 4.84E−07 | 1.75E−06 | 4.18E−06 |
| Colon Cancer | HT29 | 4.54E−07 | 1.51E−06 | 3.96E−06 |
| Colon Cancer | KM12 | 1.25E−06 | 2.88E−06 | 6.60E−06 |
| Colon Cancer | SW-620 | 2.66E−07 | — | — |
| CNS Cancer | SF-268 | 2.82E−07 | 8.53E−07 | 3.69E−06 |
| CNS Cancer | SF-295 | 1.73E−06 | 4.63E−06 | 4.94E−05 |
| CNS Cancer | SF-539 | 2.15E−07 | 5.45E−07 | 1.89E−06 |
| CNS Cancer | SNB-19 | 3.59E−07 | 1.44E−06 | 4.10E−06 |
| CNS Cancer | SNB-75 | 2.73E−07 | 9.30E−07 | 3.43E−06 |
| CNS Cancer | U251 | 1.75E−07 | 3.34E−07 | 6.40E−07 |
| Melanoma | LOX IMVI | 1.66E−07 | 3.10E−07 | 5.79E−07 |
| Melanoma | MALME−3M | 4.26E−07 | 2.11E−06 | — |
| Melanoma | M14 | 4.23E−07 | 2.03E−06 | 1.36E−05 |
| Melanoma | SK-MEL-2 | 1.07E−06 | 3.23E−06 | 9.82E−06 |
| Melanoma | SK-MEL-28 | 2.82E−07 | 6.78E−07 | — |
| Melanoma | SK-MEL-5 | 3.73E−07 | 1.47E−06 | 3.84E−06 |
| Melanoma | UACC-257 | 1.02E−06 | 2.81E−06 | 7.74E−06 |
| Melanoma | UACC62 | 5.65E−07 | 1.88E−06 | 4.88E−06 |
| Ovarian Cancer | IGROV1 | 2.72E−07 | 6.95E−07 | 3.39E−06 |
| Ovarian Cancer | OVCAR-3 | 2.01E−07 | 4.13E−07 | 8.49E−07 |
| Ovarian Cancer | OVCAR-4 | 3.48E−07 | 7.42E−06 | 3.22E−05 |
| Ovarian Cancer | OVCAR-5 | 1.46E−06 | 2.96E−06 | 5.97E−06 |
| Ovarian Cancer | OVCAR-8 | 7.59E−07 | 1.10E−05 | >1.00E−4 |
| Ovarian Cancer | SK-OV-3 | 1.65E−06 | 4.13E−06 | 1.08E−05 |
| Renal Cancer | 786-0 | 3.25E−07 | 1.07E−06 | 5.21E−06 |
| Renal Cancer | A498 | 2.50E−07 | 1.01E−06 | 4.40E−06 |
| Renal Cancer | ACHN | 7.36E−07 | 2.70E−06 | 8.56E−06 |
| Renal Cancer | CAKI-1 | 4.26E−07 | 2.79E−06 | 2.08E−05 |
| Renal Cancer | RXF 393 | 6.11E−07 | 5.92E−06 | 4.44E−05 |
| Renal Cancer | SN12C | 4.01E−07 | 6.81E−06 | 3.17E−05 |
| Renal Cancer | TK-10 | 7.16E−07 | 2.81E−06 | 9.66E−06 |
| Renal Cancer | UO-31 | 4.80E−07 | 2.08E−06 | 6.48E−06 |
| Prostate Cancer | PC-3 | 3.34E−07 | 1.82E−06 | 4.17E−05 |
| Prostate Cancer | DU-145 | 2.71E−07 | 7.06E−07 | 2.45E−06 |
| Breast Cancer | MCF7 | 2.32E−07 | 7.24E−07 | 2.61E−06 |
| Breast Cancer | NCI/ADR-RES | 2.82E−05 | >1.00E−4 | >1.00E−4 |
| Breast Cancer | MDA-MB- | 2.42E−07 | 5.84E−07 | 4.05E−05 |
| | 231/ATCC | | | |
| Breast Cancer | HS578T | 3.55E−07 | 3.69E−05 | >1.00E−4 |
| Breast Cancer | MDA-MB-435 | 3.82E−07 | 1.92E−06 | 2.31E−05 |
| Breast Cancer | BT-549 | 5.15E−07 | 6.73E−06 | >1.00E−4 |
| Breast Cancer | T-47D | 1.52E−07 | 5.72E−07 | 3.14E−06 |
| Breast Cancer | MDA-MB-468 | 2.02E−07 | 4.36E−07 | 9.41E−07 |

Biphenyl Analogue

| Panel | Cell Line | GI50 (M) | TGI (M) | LC50 (M) |
|---|---|---|---|---|
| Leukemia | CCRF-CEM | 1.05E−07 | 4.21E−07 | — |
| Leukemia | HL-60(TB) | 1.31E−07 | 6.55E−07 | 6.43E−05 |
| Leukemia | K-562 | 1.24E−07 | 3.58E−07 | >1.00E−4 |
| Leukemia | MOLT-4 | 1.59E−07 | 6.04E−07 | 7.91E−05 |
| Leukemia | RPMI-8226 | 1.02E−08 | 4.86E−08 | — |
| Leukemia | SR | 1.18E−07 | 4.07E−07 | — |
| Non-Small Cell | A549/ATCC | 2.79E−07 | 8.34E−07 | 3.00E−06 |
| Lung Cancer | | | | |
| Non-Small Cell | EKVX | 2.96E−06 | 1.11E−06 | 3.47E−06 |
| Lung Cancer | | | | |
| Non-Small Cell | HOP-62 | 1.64E−06 | 3.32E−06 | 6.74E−06 |
| Lung Cancer | | | | |
| Non-Small Cell | HOP-92 | — | — | — |
| Lung Cancer | | | | |
| Non-Small Cell | NCI-H226 | 1.99E−07 | 7.07E−07 | 2.92E−06 |
| Lung Cancer | | | | |
| Non-Small Cell | NCI-H23 | 2.06E−07 | 8.40E−07 | 3.58E−06 |
| Lung Cancer | | | | |
| Non-Small Cell | NCI-H322M | 2.78E−07 | 1.28E−06 | 3.79E−06 |
| Lung Cancer | | | | |
| Non-Small Cell | NCI-H460 | 3.26E−07 | 1.18E−06 | — |
| Lung Cancer | | | | |
| Non-Small Cell | NCI-H522 | 6.62E−08 | 2.97E−07 | 1.16E−06 |
| Lung Cancer | | | | |
| Colon Cancer | COLO 205 | 1.28E−07 | 2.59E−07 | 5.26E−07 |
| Colon Cancer | HCC-2998 | 2.69E−07 | 1.06E−06 | 3.50E−06 |
| Colon Cancer | HCT-116 | 1.86E−07 | 3.77E−07 | 7.64E−07 |
| Colon Cancer | HCT-15 | 1.46E−07 | 3.22E−07 | 7.10E−07 |
| Colon Cancer | HT29 | 2.84E−07 | >1.00E−4 | >1.00E−4 |
| Colon Cancer | KM12 | 2.57E−07 | 7.40E−07 | 3.91E−06 |
| Colon Cancer | SW-620 | 1.51E−07 | 3.49E−07 | 8.06E−07 |
| CNS Cancer | SF-268 | 2.36E−07 | 7.67E−07 | 3.32E−06 |
| CNS Cancer | SF-295 | 3.92E−07 | 1.54E−06 | 3.97E−06 |
| CNS Cancer | SF-539 | 1.86E−07 | 4.45E−07 | 1.17E−06 |
| CNS Cancer | SNB-19 | 2.38E−07 | 9.50E−07 | 3.09E−06 |
| CNS Cancer | SNB-75 | 2.42E−07 | 7.57E−07 | 3.86E−06 |
| CNS Cancer | U251 | 1.36E−07 | 2.73E−07 | 5.47E−07 |
| Melanoma | LOX IMVI | 1.39E−07 | 2.73E−07 | 5.34E−07 |
| Melanoma | MALME-3M | 4.17E−08 | 2.44E−07 | 1.50E−06 |
| Melanoma | M14 | 2.05E−07 | 5.56E−07 | 2.18E−06 |
| Melanoma | SK-MEL-2 | — | — | — |
| Melanoma | SK-MEL-28 | 2.52E−07 | 1.08E−06 | 3.77E−06 |
| Melanoma | SK-MEL-5 | 1.66E−07 | 3.24E−07 | 6.29E−07 |
| Melanoma | UACC-257 | 2.72E−07 | 1.36E−06 | 3.74E−06 |
| Melanoma | UACC62 | 1.61E−07 | 4.71E−07 | 1.85E−06 |
| Ovarian Cancer | IGROV1 | — | — | — |
| Ovarian Cancer | OVCAR-3 | 1.58E−07 | 3.95E−07 | 9.86E−07 |
| Ovarian Cancer | OVCAR-4 | 2.37E−07 | 1.72E−06 | — |
| Ovarian Cancer | OVCAR-5 | 3.68E−07 | 1.70E−06 | 4.21E−06 |
| Ovarian Cancer | OVCAR-8 | 2.77E−07 | 1.03E−06 | 3.30E−06 |
| Ovarian Cancer | SK-OV-3 | 3.00E−07 | 1.19E−06 | 3.45E−06 |
| Renal Cancer | 786-0 | 1.92E−07 | 3.98E−07 | 8.25E−07 |
| Renal Cancer | A498 | 2.85E−07 | 1.32E−06 | 3.83E−06 |
| Renal Cancer | ACHN | 2.14E−07 | 6.18E−07 | 2.26E−06 |
| Renal Cancer | CAKI-1 | — | — | — |
| Renal Cancer | RXF 393 | — | — | — |
| Renal Cancer | SN12C | 2.14E−07 | 6.14E−07 | 2.52E−06 |
| Renal Cancer | TK-10 | 2.63E−07 | 9.55E−07 | 3.38E−06 |
| Renal Cancer | UO-31 | 1.90E−07 | 7.31E−07 | 2.76E−06 |
| Prostate Cancer | PC-3 | — | — | — |
| Prostate Cancer | DU-145 | 2.80E−07 | 8.13E−07 | 3.66E−06 |
| Breast Cancer | MCF7 | 2.12E−07 | 9.84E−07 | 5.51E−06 |
| Breast Cancer | NCI/ADR-RES | 9.92E−07 | 2.27E−06 | 5.18E−06 |
| Breast Cancer | MDA-MB- | 2.40E−07 | 1.05E−06 | 3.76E−06 |
| | 231/ATCC | | | |
| Breast Cancer | HS578T | 5.46E−07 | 1.89E−05 | >1.00E−4 |
| Breast Cancer | MDA-MB-435 | 1.66E−07 | 4.40E−07 | 1.41E−06 |
| Breast Cancer | BT-549 | 9.53E−08 | 3.79E−07 | 1.70E−06 |
| Breast Cancer | T-47D | 5.76E−08 | 3.30E−07 | — |
| Breast Cancer | MDA-MB-468 | 1.60E−07 | 5.90E−07 | 3.03E−06 |

Deuterated Methanol Adduct

| Panel | Cell Line | GI50 (M) | TGI (M) | LC50 (M) |
|---|---|---|---|---|
| Leukemia | CCRF-CEM | 1.85E−07 | 7.04E−07 | 4.60E−06 |
| Leukemia | HL-60(TB) | 2.98E−07 | 1.45E−06 | 9.37E−06 |
| Leukemia | K-562 | 5.76E−07 | 3.07E−06 | >1.00E−4 |
| Leukemia | MOLT-4 | 2.29E−07 | 1.38E−06 | 8.56E−06 |
| Leukemia | RPMI-8226 | 4.02E−08 | 2.98E−07 | 3.30E−06 |
| Leukemia | SR | 2.35E−07 | 1.25E−06 | 9.37E−06 |
| Non-Small Cell | A549/ATCC | 1.56E−06 | 2.99E−06 | 5.73E−06 |
| Lung Cancer | | | | |
| Non-Small Cell | EKVX | 5.94E−07 | 1.97E−06 | 4.64E−06 |
| Lung Cancer | | | | |
| Non-Small Cell | HOP-62 | 2.39E−06 | 7.03E−06 | 2.67E−05 |
| Lung Cancer | | | | |
| Non-Small Cell | HOP-92 | 2.22E−07 | 1.30E−06 | 4.62E−06 |
| Lung Cancer | | | | |
| Non-Small Cell | NCI-H226 | 3.91E−07 | 1.70E−06 | 4.12E−06 |
| Lung Cancer | | | | |
| Non-Small Cell | NCI-H23 | 9.95E−07 | 2.43E−06 | 5.90E−06 |
| Lung Cancer | | | | |
| Non-Small Cell | NCI-H322M | 1.22E−06 | 2.54E−06 | 5.31E−06 |
| Lung Cancer | | | | |
| Non-Small Cell | NCI-H460 | 1.80E−06 | 3.60E−06 | 7.17E−06 |
| Lung Cancer | | | | |
| Non-Small Cell | NCI-H522 | 4.31E−07 | 1.80E−06 | 4.82E−06 |
| Lung Cancer | | | | |
| Colon Cancer | COLO 205 | 3.36E−07 | 1.45E−06 | 4.45E−06 |
| Colon Cancer | HCC-2998 | 6.92E−07 | 2.01E−06 | 4.71E−06 |
| Colon Cancer | HCT-116 | 7.44E−07 | 1.97E−06 | 4.44E−06 |
| Colon Cancer | HCT-15 | 5.48E−07 | 1.77E−06 | 4.23E−06 |
| Colon Cancer | HT29 | 1.26E−06 | 3.68E−06 | 6.81E−05 |
| Colon Cancer | KM12 | 1.27E−06 | 2.52E−06 | 5.02E−06 |
| Colon Cancer | SW-620 | 3.75E−07 | 1.54E−06 | 4.90E−06 |
| CNS Cancer | SF-268 | 1.50E−06 | 3.01E−06 | 6.06E−06 |
| CNS Cancer | SF-295 | 1.28E−06 | 2.73E−06 | 5.81E−06 |
| CNS Cancer | SF-539 | 4.31E−07 | 1.46E−06 | 3.83E−06 |
| CNS Cancer | SNB-19 | 1.11E−06 | 2.31E−06 | 4.81E−06 |
| CNS Cancer | SNB-75 | 2.65E−07 | 1.27E−06 | 4.72E−06 |
| CNS Cancer | U251 | 3.82E−07 | 1.37E−06 | 3.70E−06 |
| Melanoma | LOX IMVI | 9.69E−07 | 2.14E−06 | 4.63E−06 |
| Melanoma | MALME-3M | 1.74E−07 | 1.56E−06 | 5.09E−06 |
| Melanoma | M14 | 1.06E−06 | 2.50E−06 | 5.90E−06 |
| Melanoma | SK-MEL-2 | 9.01E−07 | 2.28E−06 | 5.39E−06 |
| Melanoma | SK-MEL-28 | 4.57E−07 | 1.82E−06 | 4.37E−06 |
| Melanoma | SK-MEL-5 | 1.41E−06 | 2.71E−06 | 5.23E−06 |
| Melanoma | UACC-257 | 5.87E−07 | 1.89E−06 | 4.45E−06 |
| Melanoma | UACC62 | 5.49E−07 | 1.92E−06 | 4.39E−06 |
| Ovarian Cancer | IGROV1 | 7.43E−07 | 2.04E−06 | 4.68E−06 |
| Ovarian Cancer | OVCAR-3 | 6.16E−07 | 1.81E−06 | 4.27E−06 |
| Ovarian Cancer | OVCAR-4 | 3.73E−07 | 1.63E−06 | 4.14E−06 |
| Ovarian Cancer | OVCAR-5 | 5.86E−07 | 1.91E−06 | 4.49E−06 |
| Ovarian Cancer | OVCAR-8 | 9.60E−07 | 2.17E−06 | 4.77E−06 |
| Ovarian Cancer | SK-OV-3 | 1.01E−06 | 2.16E−06 | 4.65E−06 |
| Renal Cancer | 786-0 | 1.20E−06 | 2.46E−06 | 5.02E−06 |
| Renal Cancer | A498 | 1.02E−06 | 2.22E−06 | 4.82E−06 |
| Renal Cancer | ACHN | 1.27E−06 | 2.53E−06 | 5.03E−06 |
| Renal Cancer | CAKI-1 | — | — | — |
| Renal Cancer | RXF 393 | 2.83E−07 | 6.25E−07 | 2.05E−06 |
| Renal Cancer | SN12C | 1.12E−06 | 2.32E−06 | 4.82E−06 |
| Renal Cancer | TK-10 | 1.07E−06 | 2.35E−06 | 5.15E−06 |
| Renal Cancer | UO-31 | 4.98E−07 | 1.84E−06 | 4.29E−06 |
| Prostate Cancer | PC-3 | 6.19E−07 | 2.02E−06 | 5.12E−06 |
| Prostate Cancer | DU-145 | 1.75E−06 | 3.12E−06 | 5.59E−06 |
| Breast Cancer | MCF7 | 3.48E−07 | 1.33E−06 | 4.56E−06 |
| Breast Cancer | NCI/ADR-RES | 2.61E−06 | 9.95E−06 | 3.94E−06 |
| Breast Cancer | MDA-MB- | 5.38E−07 | 1.83E−06 | 4.32E−06 |
| | 231/ATCC | | | |
| Breast Cancer | HS578T | 4.92E−07 | 2.17E−06 | — |
| Breast Cancer | MDA-MB-435 | 3.95E−07 | 1.77E−06 | 4.72E−06 |
| Breast Cancer | BT-549 | 5.98E−07 | 1.99E−06 | 4.69E−06 |
| Breast Cancer | T-47D | 1.42E−07 | 1.38E−06 | — |
| Breast Cancer | MDA-MB-468 | 2.92E−07 | 1.78E−06 | 6.96E−06 |

Glutathione Adduct

| Panel | Cell Line | GI50 (M) | TGI (M) | LC50 (M) |
|---|---|---|---|---|
| Leukemia | CCRF-CEM | 6.72E−07 | 2.03E−06 | 3.50E−05 |
| Leukemia | HL-60(TB) | 3.21E−07 | 1.62E−06 | 1.33E−05 |
| Leukemia | K-562 | 1.01E−06 | 4.66E−06 | >5.00E−5 |
| Leukemia | MOLT-4 | 4.25E−07 | 1.75E−06 | 2.06E−05 |
| Leukemia | RPMI-8226 | 1.59E−07 | 1.14E−06 | >5.00E−5 |
| Leukemia | SR | 2.28E−07 | 1.28E−06 | 1.67E−05 |
| Non-Small Cell | A549/ATCC | 1.37E−06 | 3.94E−06 | 1.58E−05 |
| Lung Cancer | | | | |
| Non-Small Cell | EKVX | 1.11E−06 | 5.40E−06 | 1.89E−05 |
| Lung Cancer | | | | |
| Non-Small Cell | HOP-62 | 6.67E−06 | 1.38E−06 | 2.86E−05 |
| Lung Cancer | | | | |
| Non-Small Cell | HOP-92 | — | — | — |
| Lung Cancer | | | | |
| Non-Small Cell | NCI-H226 | 8.02E−07 | 2.29E−06 | 8.33E−06 |
| Lung Cancer | | | | |
| Non-Small Cell | NCI-H23 | 1.09E−06 | 3.07E−06 | 1.49E−05 |
| Lung Cancer | | | | |
| Non-Small Cell | NCI-H322M | 1.38E−06 | 4.63E−06 | 1.61E−05 |
| Lung Cancer | | | | |
| Non-Small Cell | NCI-H460 | 1.11E−06 | 2.41E−06 | 2.32E−05 |
| Lung Cancer | | | | |
| Non-Small Cell | NCI-H522 | 7.27E−07 | 1.95E−06 | 6.29E−06 |
| Lung Cancer | | | | |
| Colon Cancer | COLO 205 | 6.49E−07 | 1.51E−05 | 3.51E−06 |
| Colon Cancer | HCC-2998 | 7.59E−07 | 1.51E−06 | 3.02E−06 |
| Colon Cancer | HCT-116 | 8.71E−07 | 1.76E−06 | 3.55E−06 |
| Colon Cancer | HCT-15 | 8.40E−07 | 2.04E−06 | 4.93E−06 |
| Colon Cancer | HT29 | 9.00E−07 | 2.01E−06 | 4.51E−06 |
| Colon Cancer | KM12 | 9.79E−07 | 1.91E−06 | 3.73E−06 |
| Colon Cancer | SW-620 | 7.13E−07 | 1.50E−06 | 3.81E−06 |
| CNS Cancer | SF-268 | 1.13E−06 | 2.88E−06 | 1.09E−05 |
| CNS Cancer | SF-295 | 1.45E−06 | 6.58E−06 | 2.44E−05 |
| CNS Cancer | SF-539 | 8.38E−07 | 2.03E−06 | 4.90E−06 |
| CNS Cancer | SNB-19 | 1.43E−06 | 6.24E−06 | 1.87E−05 |
| CNS Cancer | SNB-75 | 8.96E−07 | 2.45E−06 | 8.47E−06 |
| CNS Cancer | U251 | 8.19E−07 | 1.97E−06 | 4.76E−06 |
| Melanoma | LOX IMVI | 8.06E−07 | 1.56E−06 | 3.01E+00 |
| Melanoma | MALME-3M | 4.19E−07 | 3.10E−06 | 2.51E−05 |
| Melanoma | M14 | 9.96E−07 | 2.63E−06 | 1.12E−05 |
| Melanoma | SK-MEL-2 | 8.77E−07 | 2.44E−06 | 1.09E−05 |
| Melanoma | SK-MEL-28 | 9.85E−07 | 1.95E−06 | 3.87E−06 |
| Melanoma | SK-MEL-5 | 8.26E−07 | 1.86E−06 | 4.17E−06 |
| Melanoma | UACC-257 | 1.07E−06 | 5.43E−06 | 1.73E−05 |
| Melanoma | UACC62 | 6.49E−07 | 1.72E−06 | 4.56E−06 |
| Ovarian Cancer | IGROV1 | 1.06E−06 | 2.81E−06 | 1.19E−05 |
| Ovarian Cancer | OVCAR-3 | 7.35E−07 | 1.52E−06 | 3.13E−06 |
| Ovarian Cancer | OVCAR-4 | 7.42E−07 | 2.49E−06 | 1.01E−05 |
| Ovarian Cancer | OVCAR-5 | 9.71E−07 | 3.03E−06 | 1.20E−05 |
| Ovarian Cancer | OVCAR-8 | 1.60E−06 | 6.38E−06 | 2.98E−05 |
| Ovarian Cancer | SK-OV-3 | 1.30E−06 | 5.06E−06 | 1.61E−05 |
| Renal Cancer | 786-0 | 1.12E−06 | 2.44E−06 | 6.03E−06 |
| Renal Cancer | A498 | 8.04E−07 | 1.83E−06 | 4.16E−06 |
| Renal Cancer | ACHN | 9.61E−07 | 3.22E−06 | 1.23E−05 |
| Renal Cancer | CAKI-1 | — | — | — |
| Renal Cancer | RXF 393 | 8.29E−07 | 1.75E−06 | 3.69E−06 |
| Renal Cancer | SN12C | 1.51E−06 | 5.64E−06 | 1.78E−05 |
| Renal Cancer | TK-10 | 1.09E−06 | 5.46E−06 | 1.74E−05 |
| Renal Cancer | UO-31 | 7.79E−07 | 4.07E−06 | 1.50E−05 |
| Prostate Cancer | PC-3 | 9.06E−07 | 2.59E−06 | 1.16E−05 |
| Prostate Cancer | DU-145 | 1.16E−06 | 2.65E−06 | 7.88E−06 |
| Breast Cancer | MCF7 | 8.40E−07 | 3.18E−06 | 3.36E−05 |
| Breast Cancer | NCI/ADR-RES | 3.94E−06 | 1.40E−05 | 4.47E−05 |
| Breast Cancer | MDA-MB- | 9.31E−07 | 3.68E−06 | 1.47E−05 |
| | 231/ATCC | | | |
| Breast Cancer | HS578T | 1.11E−06 | 3.79E−06 | >5.00E−5 |
| Breast Cancer | MDA-MB-435 | 1.10E−06 | 5.20E−06 | 2.20E−05 |
| Breast Cancer | BT-549 | 9.23E−07 | 2.70E−06 | 1.05E−05 |
| Breast Cancer | T-47D | 3.19E−07 | 2.47E−06 | 4.60E−05 |
| Breast Cancer | MDA-MB-468 | 6.74E−07 | 1.62E−06 | 3.89E−05 |

Coenzyme A Adduct

| Panel | Cell Line | GI50 (M) | TGI (M) | LC50 (M) |
|---|---|---|---|---|
| Leukemia | CCRF-CEM | 6.98E−07 | 3.06E−06 | 2.18E−05 |
| Leukemia | HL-60(TB) | 4.08E−07 | 1.93E−06 | 2.40E−05 |
| Leukemia | K-562 | 8.84E−07 | 4.29E−06 | >3.25e−5 |
| Leukemia | MOLT-4 | 7.15E−07 | 2.47E−06 | 3.15E−05 |
| Leukemia | RPMI-8226 | 1.81E−07 | 1.38E−06 | >3.25E−5 |
| Leukemia | SR | 2.39E−07 | 1.41E−06 | 1.97E−05 |
| Non-Small Cell | A549/ATCC | 4.01E−06 | 8.60E−06 | 1.84E−05 |
| Lung Cancer | | | | |
| Non-Small Cell | EKVX | 1.73E−06 | 6.32E−06 | 1.49E−05 |
| Lung Cancer | | | | |
| Non-Small Cell | HOP-62 | 4.51E−06 | 1.31E−05 | >3.25E−5 |
| Lung Cancer | | | | |
| Non-Small Cell | HOP-92 | — | — | — |
| Lung Cancer | | | | |
| Non-Small Cell | NCI-H226 | 1.26E−07 | 1.42E−06 | 8.16E−05 |
| Lung Cancer | | | | |
| Non-Small Cell | NCI-H23 | 1.61E−06 | 6.17E−06 | 1.62E−05 |
| Lung Cancer | | | | |
| Non-Small Cell | NCI-H322M | 1.71E−06 | 6.09E−06 | 1.41E−05 |
| Lung Cancer | | | | |
| Non-Small Cell | NCI-H460 | 4.02E−06 | 8.89E−06 | 1.97E−05 |
| Lung Cancer | | | | |
| Non-Small Cell | NCI-H522 | 9.67E−07 | 5.27E−06 | 1.73E−05 |
| Lung Cancer | | | | |
| Colon Cancer | COLO 205 | 6.18E−07 | 3.07E−06 | 1.51E−05 |
| Colon Cancer | HCC-2998 | 7.76E−07 | 3.65E−06 | 1.11E−05 |
| Colon Cancer | HCT-116 | 1.31E−06 | 4.77E−06 | 1.25E−05 |
| Colon Cancer | HCT-15 | 1.03E−06 | 4.59E−06 | 1.32E−05 |
| Colon Cancer | HT29 | 1.80E−06 | 6.24E−06 | 1.58E−05 |
| Colon Cancer | KM12 | 2.21E−06 | 8.57E−06 | 2.85E−05 |
| Colon Cancer | SW-620 | 8.81E−07 | 3.57E−06 | 1.13E−05 |
| CNS Cancer | SF-268 | 2.75E−06 | 7.15E−06 | 1.66E−05 |
| CNS Cancer | SF-295 | 3.91E−06 | 8.57E−06 | 1.88E−05 |
| CNS Cancer | SF-539 | 8.91E−07 | 3.85E−06 | 1.12E−05 |
| CNS Cancer | SNB-19 | 3.11E−06 | 7.89E−06 | 1.94E−05 |
| CNS Cancer | SNB-75 | 1.12E−06 | 4.74E−06 | 1.28E−05 |
| CNS Cancer | U251 | 9.92E−07 | 4.10E−06 | 1.17E−05 |
| Melanoma | LOX IMVI | 1.04E−06 | 4.08E−06 | 1.19E−05 |
| Melanoma | MALME-3M | 2.67E−07 | 5.24E−06 | 1.94E−05 |
| Melanoma | M14 | 2.62E−06 | 7.27E−06 | 1.76E−05 |
| Melanoma | SK-MEL-2 | 1.93E−06 | 7.47E−06 | 2.15E−05 |
| Melanoma | SK-MEL-28 | 9.80E−07 | 4.30E−06 | 1.26E−05 |
| Melanoma | SK-MEL-5 | 2.62E−06 | 6.86E−06 | 1.54E−05 |
| Melanoma | UACC-257 | 1.25E−06 | 5.62E−06 | 1.51E−05 |
| Melanoma | UACC62 | 4.49E−07 | 3.74E−06 | 1.15E−05 |
| Ovarian Cancer | IGROV1 | 1.14E−06 | 4.87E−06 | 1.42E−05 |
| Ovarian Cancer | OVCAR-3 | 9.11E−07 | 3.51E−06 | 1.11E−05 |
| Ovarian Cancer | OVCAR-4 | 1.52E−06 | 5.30E−06 | 1.34E−05 |
| Ovarian Cancer | OVCAR-5 | 3.27E−06 | 7.11E−06 | 1.54E−05 |
| Ovarian Cancer | OVCAR-8 | 2.10E−06 | 6.63E−06 | 1.66E−05 |
| Ovarian Cancer | SK-OV-3 | 2.47E−06 | 7.05E−06 | 1.69E−05 |
| Renal Cancer | 786-0 | 3.21E−06 | 7.05E−06 | 1.53E−05 |
| Renal Cancer | A498 | 6.21E−07 | 2.82E−06 | 9.91E−06 |
| Renal Cancer | ACHN | 1.63E−06 | 5.79E−06 | 1.37E−05 |
| Renal Cancer | CAKI-1 | — | — | — |
| Renal Cancer | RXF 393 | 8.55E−07 | 2.36E−06 | 1.00E−05 |
| Renal Cancer | SN12C | 1.11E−06 | 5.58E−06 | 1.36E−05 |
| Renal Cancer | TK-10 | 5.51E−06 | 7.65E−06 | 1.67E−05 |
| Renal Cancer | UO-31 | 1.01E−06 | 4.98E−06 | 1.31E−05 |
| Prostate Cancer | PC-3 | 1.23E−06 | 5.13E−06 | 1.48E−05 |
| Prostate Cancer | DU-145 | 4.68E−06 | 9.05E−06 | 1.75E−05 |
| Breast Cancer | MCF7 | 6.99E−07 | 3.00E−06 | 1.30E−05 |
| Breast Cancer | NCI/ADR-RES | 4.91E−06 | 1.39E−05 | >3.25E−5 |
| Breast Cancer | MDA-MB- | 6.74E−07 | 3.83E−06 | 1.17E−05 |
| | 231/ATCC | | | |
| Breast Cancer | HS578T | 5.16E−07 | 2.21E−06 | 1.73E−05 |
| Breast Cancer | MDA-MB-435 | 9.30E−07 | 4.32E−06 | 1.35E−05 |
| Breast Cancer | BT-549 | 1.51E−06 | 5.82E−06 | 1.46E−05 |
| Breast Cancer | T-47D | 4.48E−07 | 3.85E−06 | 2.13E−05 |
| Breast Cancer | MDA-MB-468 | 7.74E−07 | 2.91E−06 | 1.10E−05 |

### Other Embodiments

The foregoing has been a description of certain non-limiting preferred embodiments of the invention. Those of ordinary skill in the art will appreciate that various changes and modifications to this description may be made without departing from the spirit or scope of the present invention, as defined in the following claims.

1. A compound of formula: 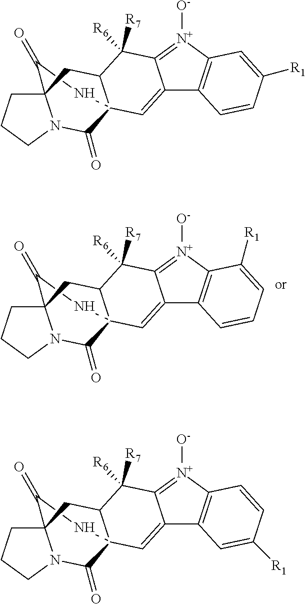
wherein
R₁, R₆, and R₇ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —C(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety.

2. The compound of claim 1, wherein R₁ is substituted or unsubstituted aryl.

3. The compound of claim 1, wherein R₁ is substituted or unsubstituted phenyl.

4. The compound of claim 1, wherein R₁ is unsubstituted phenyl.

5. The compound of claim 1, wherein R₁ is arylalkenyl or arylalkynyl.

6. The compound of claim 1, wherein R₁ is phenylalkenyl or phenylalkynyl.

7. The compound of claim 1, wherein R₆ and R₇ are each independently hydrogen or C₁₋₆ alkyl.

8. The compound of claim 1, wherein both R₆ and R₇ are methyl.

9. The compound of claim 1 of formula:

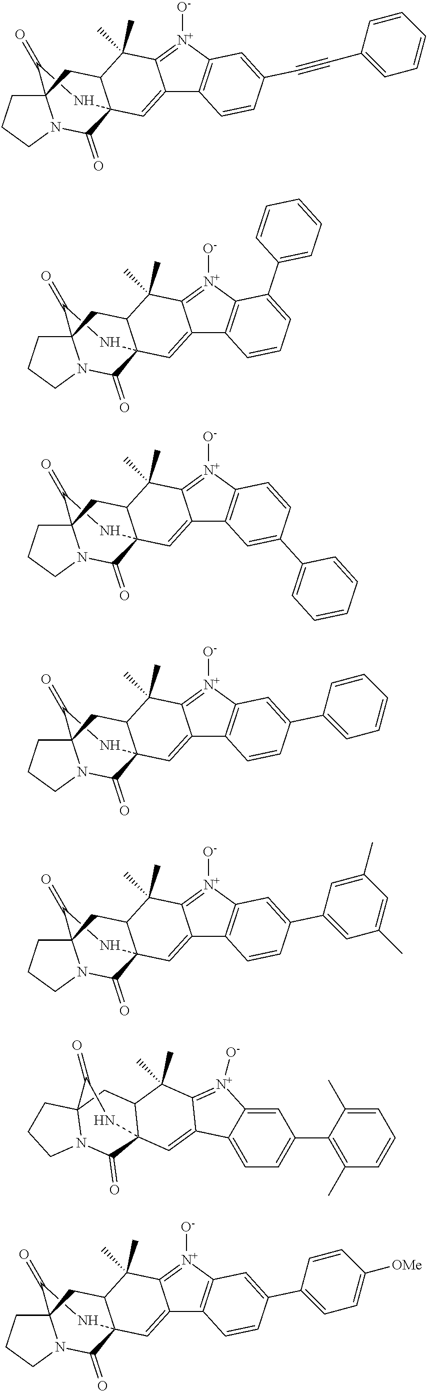

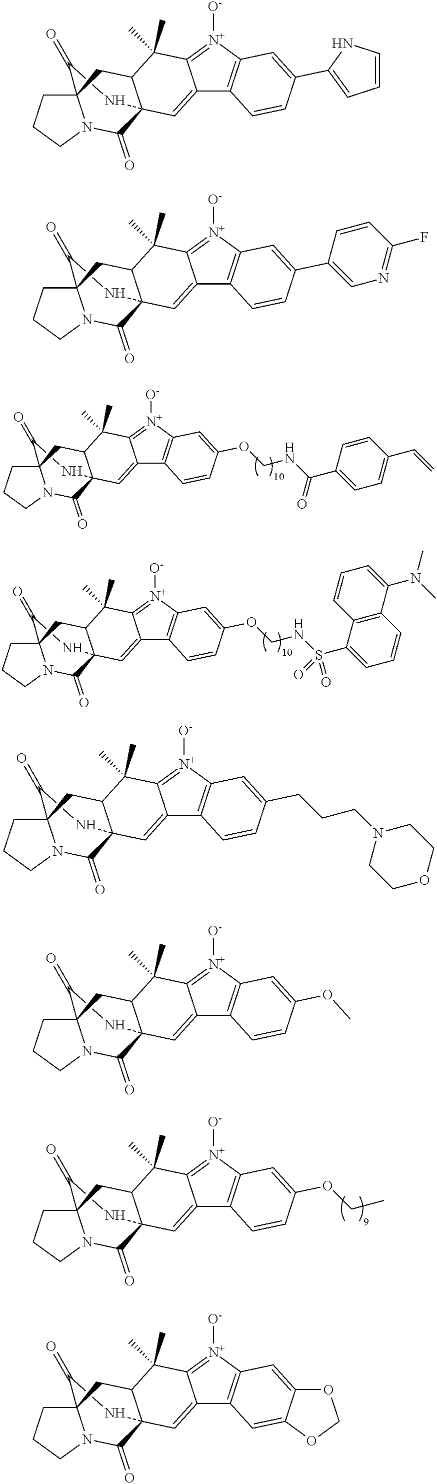

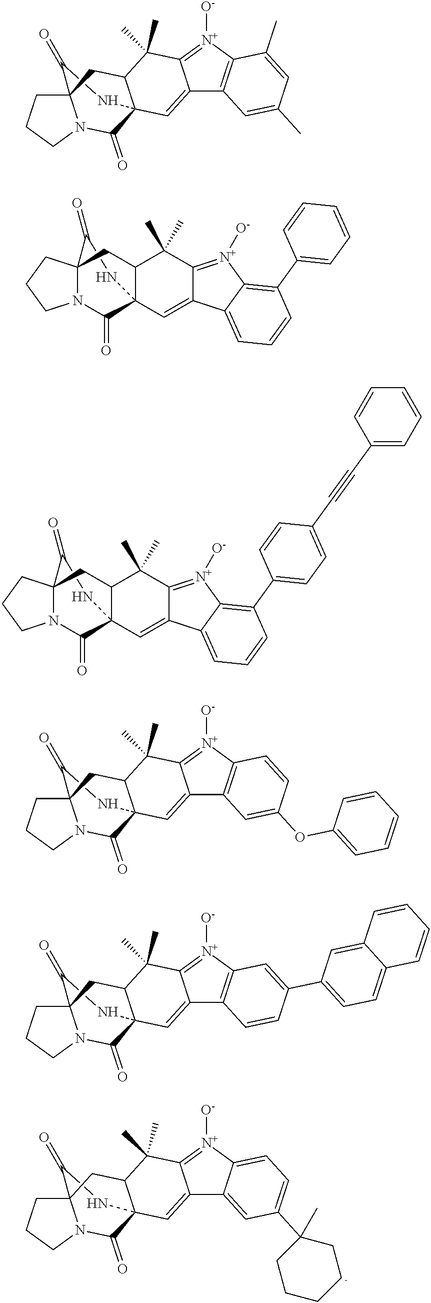

10.-23. (canceled)

24. A pharmaceutical composition comprising a compound of claim 1 and a pharmaceutically acceptable excipient.

25. A method of modifying nucleophosmin, the method comprising steps of:
contacting an avrainvillamide analogue of formula: 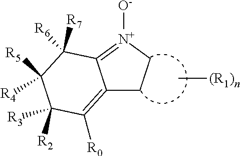
wherein R₀, R₁, R₂, R₃, R₄, R₅, R₆, and R₇ are independently selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR_{G}; —C(═O)R_{G}; —CO₂R_{G}; —CN; —SCN; —SR_{G}; —SOR_{G}; —SO₂R_{G}; —NO₂; —N₃; —N(R_{G})₂; —NHC(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;
wherein two or more substituents may form substituted or unsubstituted, cyclic, heterocyclic, aryl, or heteroaryl structures;
wherein R₂ and R₃, R₄ and R₅, or R₆ and R₇ may form together ═O, ═NR_{G}, or ═C(R_{G})₂, wherein each occurrence of R_{G} is defined as above; 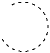
represents a substituted or unsubstituted, cyclic, heterocyclic, aryl, or heteroaryl ring system; and
n is an integer between 0 and 4;
under suitable conditions for the avrainvillamide analogue to bind nucleophosmin.

26. The method of claim 25 whereby nucleophosmin is covalently modified by the avrainvillamide analogue.

27.-32. (canceled)

33. The method of claim 25, wherein the step of contacting is done outside a cell.

34. The method of claim 25, wherein the step of contacting modulates the expression or activity of a nucleophosmin-binding protein.

35. The method of claim 25, wherein the step of contacting modulates the expression or activity of p53.

36. The method of claim 25, wherein the step of contacting modulates the expression or activity of hDM2/mDM2.

37. The method of claim 25, wherein the step of contacting modulates the expression or activity of p14ARF/p19ARF.

38. The method of claim 25, wherein the step of contacting modulates nucelophosmin's ability to act as a histone chaperone.

39. The method of claim 25, wherein the step of contacting modulates nucelophosmin's ability to act as a polynucleotide binder.

40. The method of claim 25, wherein the step of contacting modulates nucleophosmin's oligomerization state.

41.-69. (canceled)
